(19) United States
(12) Patent Application Publication
(10) Pub. No.: US 2005/0239826 A1
(43) Pub. Date: Oct. 27, 2005

(54) Alkyne compounds with MCH antagonistic activity and medicaments comprising these compounds

(75) Inventors: Dirk Stenkamp, Biberach (DE); Stephan Georg Mueller, Warthausen (DE); Philipp Lustenberger, Warthausen (DE); Thorsten Lehmann-Lintz, Ochsenhausen (DE); Gerald Juergen Roth, Biberach (DE); Klaus Rudolf, Warthausen (DE); Marcus Schindler, Biberach (DE); Leo Thomas, Biberach (DE); Ralf R.H. Lotz, Schemmerhofen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/104,915

(22) Filed: Apr. 13, 2005

Related U.S. Application Data
(60) Provisional application No. 60/563,677, filed on Apr. 20, 2004.

(30) Foreign Application Priority Data
Apr. 14, 2004 (DE) 10 2004 017 935.2

(51) Int. Cl. A61K 31/4709; A61K 31/44; A61K 31/4439; C07D 45/02; C07D 43/02

(52) U.S. Cl. 514/314; 514/357; 514/339; 546/176; 546/277.4; 546/329

(57) ABSTRACT

The present invention relates to alkyne compounds of general formula I 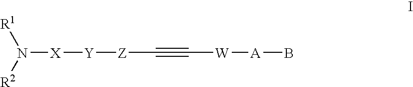
wherein the groups and radicals A, B, W, X, Y, Z, R¹ and R² have the meanings given in claim 1. Moreover the invention relates to pharmaceutical compositions containing at least one alkyne according to the invention. By virtue of their MCH-receptor antagonistic activity the pharmaceutical compositions according to the invention are suitable for the treatment of metabolic disorders and/or eating disorders, particularly obesity and diabetes.

The present invention relates to new alkyne compounds, the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention also relates to the use of a compound according to the invention for influencing eating behaviour and for reducing body weight and/or for preventing any increase in body weight in a mammal. It further relates to compositions and medicaments containing a compound according to the invention and processes for preparing them. Other aspects of this invention relate to processes for preparing the compounds according to the invention.

### BACKGROUND TO THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialised countries, have promoted morbid overweight (also known as corpulence or obesity) in recent decades. In affected people, obesity leads directly to restricted mobility and a reduction in the quality of life. There is the additional factor that obesity often leads to other diseases such as, for example, diabetes, dyslipidaemia, high blood pressure, arteriosclerosis and coronary heart disease. Moreover, high body weight alone puts an increased strain on the support and mobility apparatus, which can lead to chronic pain and diseases such as arthritis or osteoarthritis. Thus, obesity is a serious health problem for society.

The term obesity means an excess of adipose tissue in the body. In this connection, obesity is fundamentally to be seen as the increased level of fatness which leads to a health risk. There is no sharp distinction between normal individuals and those suffering from obesity, but the health risk accompanying obesity is presumed to rise continuously as the level of fatness increases. For simplicity's sake, in the present invention, individuals with a Body Mass Index (BMI), which is defined as the body weight measured in kilograms divided by the height (in metres) squared, above a value of 25 and more particularly above 30, are preferably regarded as suffering from obesity.

Apart from physical activity and a change in nutrition, there is currently no convincing treatment option for effectively reducing body weight. However, as obesity is a major risk factor in the development of serious and even life-threatening diseases, it is all the more important to have access to pharmaceutical active substances for the prevention and/or treatment of obesity. One approach which has been proposed very recently is the therapeutic use of MCH antagonists (cf. inter alia WO 01/21577, WO 01/82925).

Melanin-concentrating hormone (MCH) is a cyclic neuropeptide consisting of 19 amino acids. It is synthesised predominantly in the hypothalamus in mammals and from there travels to other parts of the brain by the projections of hypothalamic neurones. Its biological activity is mediated in humans through two different G-protein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e. changing metabolic activity and food intake [1,2]. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH mRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are presumably mediated in rodents through the G_{αs}-coupled MCH-1R [3-6], as, unlike primates, ferrets and dogs, no second MCH receptor subtype has hitherto been found in rodents. After losing the MCH-1R, knock-out mice have a lower fat mass, an increased energy conversion and, when fed on a high fat diet, do not put on weight, compared with control animals. Another indication of the importance of the MCH system in regulating the energy balance results from experiments with a receptor antagonist (SNAP-7941) [3]. In long term trials the animals treated with the antagonist lose significant amounts of weight.

In addition to its anorectic effect, the MCH-1R antagonist SNAP-7941 also achieves additional anxiolytic and antidepressant effects in behavioural experiments on rats [3]. Thus, there are clear indications that the MCH-MCH-1R system is involved not only in regulating the energy balance but also in affectivity.

Literature:

1. Qu, D., et al., A role for melanin-concentrating hormone in the central regulation of feeding behaviour. Nature, 1996. 380(6571): p. 243-7.
2. Shimada, M., et al., Mice lacking melanin-concentrating hormone are hypophagic and lean. Nature, 1998. 396(6712): p. 670-4.
3. Borowsky, B., et al., Antidepressant, anxiolytic and anorectic effects of a melanin-concentrating hormone-1 receptor antagonist. Nat Med, 2002. 8(8): p. 825-30.
4. Chen, Y., et al., Targeted disruption of the melanin-concentrating hormone receptor-1 results in hyperphagia and resistance to diet-induced obesity. Endocrinology, 2002. 143(7): p.2469-77.
5. Marsh, D. J., et al., Melanin-concentrating hormone 1 receptor-deficient mice are lean, hyperactive, and hyperphagic and have altered metabolism. Proc Natl Acad Sci USA, 2002. 99(5): p. 3240-5.
6. Takekawa, S., et al., T-226296: A novel, orally active and selective melanin-concentrating hormone receptor antagonist. Eur J Pharmacol, 2002. 438(3): p. 129-35.

In the patent literature certain amine compounds are proposed as MCH antagonists. Thus, WO 01/21577 (Takeda) describes compounds of formula 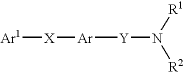
wherein Ar¹ denotes a cyclic group, X denotes a spacer, Y denotes a bond or a spacer, Ar denotes an aromatic ring which may be fused with a non-aromatic ring, R¹ and R² independently of one another denote H or a hydrocarbon group, while R¹ and R² together with the adjacent N atom may form an N-containing hetero ring and R² with Ar may also form a spirocyclic ring, R together with the adjacent N atom and Y may form an N-containing hetero ring, as MCH antagonists for the treatment of obesity.

Moreover WO 01/82925 (Takeda) also describes compounds of formula 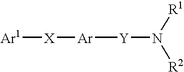
wherein Ar¹ denotes a cyclic group, X and Y represent spacer groups, Ar denotes an optionally substituted fused polycyclic aromatic ring, R¹ and R² independently of one another represent H or a hydrocarbon group, while R¹ and R² together with the adjacent N atom may form an N-containing heterocyclic ring and R² together with the adjacent N atom and Y may form an N-containing hetero ring, as MCH antagonists for the treatment of obesity, inter alia.

The as yet unpublished International Application PCT/EP0311887, which describes alkyne compounds as MCH-antagonists, also mentions the following substances, inter alia:
(2-{4-[5-(4-chloro-phenyl)-pyridin-2-ylethynyl]-2-methyl-phenoxy}-ethyl)-methyl-prop-2-ynyl-amine,
(2-{5-[5-(4-chloro-phenyl)-pyridin-2-ylethynyl]-indol-1-yl}-ethyl)-cyclopropylmethyl-prop-2-ynyl-amine,
{4-[6-(4-chloro-phenyl)-quinolin-2-ylethynyl]-benzyl}-methyl-(tetrahydro-pyran-4-yl)-amine,
allyl-(2-{4-[5-(4-chloro-phenyl)-pyridin-2-ylethynyl]-phenoxy}-ethyl)-cyclopropylmethyl-amine,
allyl-(2-{4-[5-(4-chloro-phenyl)-pyridin-2-ylethynyl]-2-methyl-phenoxy}-ethyl)-cyclopropylmethyl-amine,
allyl-(2-{5-[5-(4-chloro-phenyl)-pyridin-2-ylethynyl]-indol-1-yl}-ethyl)-cyclopropylmethyl-amine.

### AIM OF THE INVENTION

The aim of the present invention is to identify new alkyne compounds, particularly those which are especially effective as MCH antagonists. The invention also sets out to provide new alkyne compounds which can be used to influence the eating habits of mammals and achieve a reduction in body weight, particularly in mammals, and/or prevent an increase in body weight.

The present invention further sets out to provide new pharmaceutical compositions which are suitable for the prevention and/or treatment of symptoms and/or diseases caused by MCH or otherwise causally connected to MCH. In particular, the aim of this invention is to provide pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the amide compounds according to the invention. Other aims of the present invention will be immediately apparent to the skilled man from the foregoing remarks and those that follow.

### OBJECT OF THE INVENTION

In a first aspect the present invention relates to alkyne compounds of general formula I 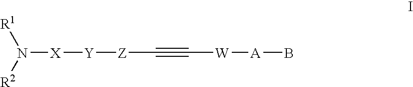
wherein
R¹ denotes C₃₋₆-alkenyl, C₃₋₆-alkynyl, (hydroxy-C₃₋₇-cycloalkyl)-C₁₋₃-alkyl, oxa-C₄₋₇-cycloalkyl, dihydroxy-C₃₋₇-alkyl,
   while the groups mentioned may be mono- or polysubstituted substituted by substituents which are selected independently of one another from the group consisting of halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino and di-(C₁₋₄-alkyl)-amino, while the alkyl, alkoxy, cylcoalkyl groups may comprise one or more identical or different substituents selected from halogen and hydroxy; and
R² independently of R¹ has one of the meanings given hereinbefore for R¹ or R² has a meaning from the group consisting of H, C₁₋₈-alkyl, C₃₋₇-cycloalkyl or a phenyl or pyridinyl group optionally mono- or polysubstituted by identical or different groups R²⁰ and/or monosubstituted by nitro, while the alkyl or cycloalkyl group may be mono- or polysubstituted by identical or different groups R¹¹, and a —CH₂— group in position 3 or 4 of a 5-, 6- or 7-membered cycloalkyl group may be replaced by —O—, —S— or —NR¹³—, or
the groups R¹, R² together with the N atom to which they are bound form a heterocyclic group which selected is from the meanings
   dihydroxy-cyclo-C₄₋₇-alkylene-imino,
   (hydroxy-C₁₋₄-alkyl)-hydroxy-cyclo-C₃₋₇-alkylene-imino,
   (hydroxy-C₁₋₃-alkyl)-cyclo-C₃₋₇-alkylene-imino, while in the last definition the C₁₋₃-alkyl group is substituted by one or more identical or different C₁₋₃-alkyl groups which may be joined together to form a C₃₋₇-cyloalkyl group;
   while the above-mentioned heterocyclic groups may be mono- or polysubstituted by substituents which are selected independently of one another from the group consisting of halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino and di-(C₁₋₄-alkyl)-amino, while alkyl, alkoxy, cylcoalkyl groups may comprise one or more identical or different substituents selected from halogen and hydroxy;
x denotes a C₁₋₄-alkylene bridge, while in the definition C₂₋₄-alkylene one or two C atoms may be monosubstituted by R¹⁰, or
   a C₃₋₄-alkylene bridge, wherein a —CH₂—CH₂— group not directly adjacent to the N atom of the R¹R²N— group is replaced by —CH═CH—, —C≡C—, —CH₂—O—, —CH₂—S— or —CH₂—NR⁴—,
   while the meanings given for X hereinbefore may comprise a substituent selected from C₂₋₆-alkenyl, C₂₋₆-alkynyl, C₃₋₇-cycloalkyl and C₃₋₇-cycloalkyl-C₁₋₃-alkyl as well as independently one, two or three identical or different C₁₋₄-alkyl substituents, while two alkyl groups may be joined together forming a 3 to 7-membered or an alkyl and an alkenyl group may be joined together forming a 5 to 7-membered cyclic group, and
W, Z independently of one another denote a single bond or a C₁₋₂-alkylene bridge,
   while two adjacent C atoms may be joined together with an additional C₁₋₄-alkylene bridge, and
   one or two C atoms independently of one another may be substituted by one or two identical or different C₁₋₃-alkyl groups, while two alkyl groups may be joined together to form a carbocyclic ring, and
Y, A are selected independently of one another from the group of the bivalent cyclic groups phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydro-isoquinolinyl, benzimidazolyl, benzoxazolyl, chromanyl, chromen-4-onyl, thienyl, furanyl, benzothienyl or benzofuranyl, while the above-mentioned cyclic groups may be mono- or polysubstituted at one or more C atoms by identical or different groups R²⁰, and in the case of a phenyl ring may also additionally be monosubstituted by nitro, and/or one or more NH groups may be substituted by R²¹,
   B has one of the meanings given for Y, A or denotes
      C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl or C₃₋₇-cycloalkyl-C₁₋₃-alkynyl, wherein one or more C atoms may be mono- or polysubstituted independently of one another by halogen and/or monosubstituted by hydroxy or cyano and/or cyclic groups may be mono- or polysubstituted by identical or different groups R²⁰,
   Cy denotes a carbo- or heterocyclic group selected from one of the following meanings
      a saturated 3- to 7-membered carbocyclic group,
      an unsaturated 4- to 7-membered carbocyclic group,
      a phenyl group,
      a saturated 4- to 7-membered or unsaturated 5- to 7-membered heterocyclic group with an N, O or S atom as heteroatom,
      a saturated or unsaturated 5- to 7-membered heterocyclic group with two or more N atoms or with one or two N atoms and an O or S atom as heteroatoms,
      an aromatic heterocyclic 5- or 6-membered group with one or more identical or different heteroatoms selected from N, O and/or S,
   while the above-mentioned saturated 6- or 7-membered groups may also be present as bridged ring systems with an imino, (C₁₋₄-alkyl)-imino, methylene, (C₁₋₄-alkyl)-methylene or di-(C₁₋₄-alkyl)-methylene bridge, and
   the above-mentioned cyclic groups may be mono- or polysubstituted at one or more C atoms by identical or different groups R²⁰, in the case of a phenyl group may also additionally be monosubstituted by nitro, and/or one or more NH groups may be substituted by R²¹,
R⁴ denotes H, C₁₋₄-alkyl, C₃₋₇-cycloalkyl or C₃₋₇-cycloalkyl-C₁₋₃-alkyl,
R¹⁰ denotes hydroxy, ω-hydroxy-C₁₋₃-alkyl, C₁₋₄-alkoxy or C₁₋₄-alkoxy-C₁₋₃-alkyl,
R¹¹ denotes halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N, R¹⁸R¹⁹N—CO— or Cy, while in the above-mentioned groups one or more C atoms may be mono- or polysubstituted independently of one another by substituents selected from halogen, OH, CN, CF₃, C₁₋₃-alkyl, hydroxy-C₁₋₃-alkyl;
R¹³ has one of the meanings given for R¹⁷,
R¹⁵ denotes H, C₁₋₄-alkyl, C₃₋₇-cycloalkyl, C₃₋₇-cycloalkyl-C₁₋₃-alkyl, phenyl, phenyl-C₁₋₃-alkyl, pyridinyl or pyridinyl-C₁₋₃-alkyl,
R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl or cyclo-C₃₋₆-alkyleneimino-C₂₋₆-alkyl,
R¹⁷ has one of the meanings given for R¹⁶ or denotes phenyl, phenyl-C₁₋₃-alkyl, pyridinyl, C₁₋₄-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylsulphonyl, C₁₋₄-alkylsulphonylamino-C₂₋₃-alkyl or N—(C₁₋₄-alkylsulphonyl)-N(-C₁₋₄-alkyl)-amino-C₂₋₃-alkyl;
R¹⁸, R¹⁹ independently of one another denote H or C₁₋₆-alkyl,
R²⁰ denotes halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl or has one of the meanings given for R²²,
R²¹ denotes C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulphonyl, aminosulphonyl, C₁₋₄-alkylaminosulphonyl, di-C₁₋₄-alkylaminosulphonyl or cyclo-C₃₋₆-alkylene-imino-sulphonyl,
R²² denotes pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulphonyl, C₁₋₄-alkyl-sulphinyl, C₁₋₄-alkyl-sulphonylamino, amino, C₁₋₄-alkylamino, di-(C₁₋₄-alkyl)-amino, C₁₋₄-alkyl-carbonyl-amino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethyl-amino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino or C₁₋₄-alkylaminocarbonylamino,
   while in the above-mentioned groups and radicals, particularly in W, X, Z, R⁴, R¹⁰, R¹³ and R¹⁵ to R²², in each case one or more C atoms may additionally be mono- or polysubstituted by F and/or in each case one or two C atoms independently of one another may additionally be monosubstituted by Cl or Br and/or in each case one or more phenyl rings may additionally comprise independently of one another one, two or three substituents selected from the group F, Cl, Br, I, cyano, C₁₋₄-alkyl, C₁₋₄-alkoxy, difluoromethyl, trifluoromethyl, hydroxy, amino, C₁₋₃-Aalkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl- and di-(C₁₋₃-alkyl)-amino-C₁₋₃-alkyl and/or may be monosubstituted by nitro, and
   the H atom of any carboxy group present or an H atom bound to an N atom may in each case be replaced by a group which can be cleaved in vivo,
the tautomers, the diastereomers, the enantiomers, the mixtures thereof and the salts thereof,
while the following compounds are not included in the invention:
(2-{4-[5-(4-chloro-phenyl)-pyridin-2-ylethynyl]-2-methyl-phenoxy}-ethyl)-methyl-prop-2-ynyl-amine,
(2-{5-[5-(4-chloro-phenyl)-pyridin-2-ylethynyl]-indol-1-yl}-ethyl)-cyclopropylmethyl-prop-2-ynyl-amine,
{4-[6-(4-chloro-phenyl)-quinolin-2-ylethynyl]-benzyl}-methyl-(tetrahydro-pyran-4-yl)-amine,
allyl-(2-{4-[5-(4-chloro-phenyl)-pyridin-2-ylethynyl]-phenoxy}-ethyl)-cyclopropylmethyl-amine,
allyl-(2-{4-[5-(4-chloro-phenyl)-pyridin-2-ylethynyl]-2-methyl-phenoxy}-ethyl)-cyclopropylmethyl-amine,
allyl-(2-{5-[5-(4-chloro-phenyl)-pyridin-2-ylethynyl]-indol-1-yl}-ethyl)-cyclopropylmethyl-amine.

The compounds according to the present invention, including the physiologically acceptable salts, are especially effective, compared with known, structurally similar compounds, as antagonists of the MCH receptor, particularly the MCH-1 receptor, and exhibit very good affinity in MCH receptor binding studies. In addition, the compounds according to the invention have a high to very high selectivity with regard to the MCH receptor. Generally the compounds according to the invention have low toxicity, they are well absorbed by oral route and have good intracerebral transitivity, particularly brain accessibility.

The invention also relates to the compounds in the form of the individual optical isomers, mixtures of the individual enantiomers or racemates, in the form of the tautomers and in the form of the free bases or corresponding acid addition salts with pharmacologically acceptable acids. The subject of the invention also includes the compounds according to the invention, including their salts, wherein one or more hydrogen atoms are replaced by deuterium.

This invention also includes the physiologically acceptable salts of the alkyne compounds according to the invention as described above and hereinafter.

Also covered by this invention are compositions containing at least one alkyne compound according to the invention and/or a salt according to the invention optionally together with one or more physiologically acceptable excipients.

Also covered by this invention are pharmaceutical compositions containing at least one alkyne compound according to the invention and/or a salt according to the invention optionally together with one or more inert carriers and/or diluents.

This invention also relates to the use of at least one alkyne compound according to the invention and/or a salt according to the invention for influencing the eating behaviour of a mammal.

The invention further relates to the use of at least one alkyne compound according to the invention and/or a salt according to the invention for reducing the body weight and/or for preventing an increase in the body weight of a mammal.

The invention also relates to the use of at least one alkyne compound according to the invention and/or a salt according to the invention for preparing a pharmaceutical composition with an MCH receptor-antagonistic activity, particularly with an MCH-1 receptor-antagonistic activity.

This invention also relates to the use of at least one alkyne compound according to the invention and/or a salt according to the invention for preparing a pharmaceutical composition which is suitable for the prevention and/or treatment of symptoms and/or diseases which are caused by MCH or are otherwise causally connected with MCH.

A further object of this invention is the use of at least one alkyne compound according to the invention and/or a salt according to the invention for preparing a pharmaceutical composition which is suitable for the prevention and/or treatment of metabolic disorders and/or eating disorders, particularly obesity, bulimia, bulimia nervosa, cachexia, anorexia, anorexia nervosa and hyperphagia.

The invention also relates to the use of at least one alkyne compound according to the invention and/or a salt according to the invention for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis.

In addition the present invention relates to the use of at least one alkyne compound according to the invention and/or a salt according to the invention for preparing a pharmaceutical composition which is suitable for the prevention and/or treatment of hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders.

The invention also relates to the use of at least one alkyne compound according to the invention and/or a salt according to the invention for preparing a pharmaceutical composition which is suitable for the prevention and/or treatment of urinary problems, such as for example urinary incontinence, overactive bladder, urgency, nycturia and enuresis.

The invention further relates to processes for preparing for preparing a pharmaceutical composition according to the invention, characterised in that at least one alkyne compound according to the invention and/or a salt according to the invention is incorporated in one or more inert carriers and/or diluents by a non-chemical method.

The invention also relates to a pharmaceutical composition containing a first active substance which is selected from the alkyne compounds according to the invention and/or the corresponding salts as well as a second active substance which is selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of hyperlipidaemia, including arteriosclerosis, active substances for the treatment of arthritis, active substances for the treatment of anxiety states and active substances for the treatment of depression, optionally together with one or more inert carriers and/or diluents.

Moreover, in one aspect, the invention relates to a process for preparing alkyne compounds of formula A.5
R¹R²N—X—Y—C≡C—W-A-B (A.5)
while in formulae A.1, A.2, A.3, A.4 and A.5 R¹, R², X, Y, W, A and B have one of the meanings given hereinbefore and hereinafter,
wherein a halogen compound of formula A.1
   HO—X—Y-Hal (A.1)
wherein Hal denotes chlorine, bromine or iodine, preferably bromine or iodine, is reacted with an alkyne compound of formula A.2
   H—C≡C—W-A-B (A.2)
in the presence of a suitable palladium catalyst, a suitable base and copper(I)iodide in a suitable solvent, and
the compound of formula A.3 obtained
   HO—X—Y—C≡C—W-A-B (A.3)
is reacted with methanesulphonic acid chloride (MsCl) to produce the methanesulphonate derivative A.4,
   MsO—X—Y—C≡C—W-A-B (A.4)
which is further reacted with an amine of formula H—NR¹R² to form the end product A.5.

This invention further relates to a process for preparing alkyne compounds of formula B.5
R¹R²N—X—Y-Z-C≡C-A-B (B.5)
while in formulae B.1, B.2, B.3, B.4 and B.5 R¹, R², X, Y, Z, A and B have one of the meanings given hereinbefore and hereinafter,
wherein a halogen compound of formula B.1
   Hal-A-B (B.1)
wherein Hal denotes chlorine, bromine or iodine, preferably bromine or iodine, is reacted with an alkyne compound of formula B.2
   HO—X—Y-Z-C≡C—H (B.2)
in the presence of a suitable palladium catalyst, a suitable base and copper(I)iodide in a suitable solvent, and
the resulting compound of formula B.3
   HO—X—Y-Z-C≡C-A-B (B.3)
is reacted with methanesulphonic acid chloride (MsCl) to form the methanesulphonate derivative B.4,
   MsO—X—Y-Z-C≡C-A-B (B.4)
which is reacted further with an amine of formula H—NR¹R² to form the end product B.5.

In addition, the invention relates to a process for preparing alkyne compounds of formula C.3
R¹R²N—X—Y—C≡C—W-A-B (C.3)
while in formulae C.1, C.2 and C.3 R¹, R², X, Y, W, A and B have one of the meanings given hereinbefore and hereinafter,
wherein a halogen compound of formula C.1
   R¹R²N—X—Y-Hal (C.1)
wherein Hal denotes chlorine, bromine or iodine, preferably bromine or iodine, is further reacted with an alkyne compound of formula C.2
   H—C≡C—W-A-B (C.2)
in the presence of a suitable palladium catalyst, a suitable base and copper(I)iodide in a suitable solvent to yield the end product C.3.

In another aspect the invention relates to a process for preparing alkyne compounds of formula D.3
R¹R²N—X—Y-Z-C≡C-A-B (D.3)
while in formulae D.1, D.2 and D.3 R¹, R², X, Y, Z, A and B have one of the meanings given hereinbefore and hereinafter,
wherein a halogen compound of formula D.2
   Hal-A-B (D.2)
wherein Hal denotes chlorine, bromine or iodine, preferably bromine or iodine, is reacted with an alkyne compound of formula D.1
   R¹R²N—X—Y-Z-C≡C—H (D.1)
in the presence of a suitable palladium catalyst, a suitable base and copper(I)iodide in a suitable solvent to form the end product D.3.

### DETAILED DESCRIPTION OF THE INVENTION

Unless otherwise specified, the groups, residues and substituents, particularly A, B, W, X, Y, Z, Cy, R¹, R², R⁴, R¹⁰, R¹¹, R¹³ and R¹⁵ to R²², have the meanings given hereinbefore.

If groups, residues and/or substituents occur more than once in a compound, they may have the same or different meanings in each case.

According to a first embodiment the group R¹ is selected from among the definitions C₃₋₆-alkenyl, C₃₋₆-alkynyl, (hydroxy-C₃₋₇-cycloalkyl)-C₁₋₃-alkyl, oxa-C₅₋₇-cycloalkyl, dihydroxy-C₃₋₇-alkyl, while the groups mentioned may be mono- or polysubstituted by substituents which are selected independently of one another from the group consisting of halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino and di-(C₁₋₄-alkyl)-amino, while any alkyl, alkoxy, cylcoalkyl groups present in the above-mentioned substituents may independently of one another comprise one or more identical or different substituents selected from halogen, particularly fluorine or chlorine, and hydroxy.

According to this embodiment preferred meanings of the group R¹ are C₃₋₅-alk-2-enyl, C₃₋₅-alk-2-inyl, (1-hydroxy-C₃₋₆-cycloalkyl)-C₁₋₃-alkyl, tetrahydropyran-3-yl, tetrahydropyran-4-yl, dihydroxy-C₃₋₅-alkyl, while the groups mentioned may be mono- or polysubstituted as specified hereinbefore.

According to this first embodiment preferred substituents, independently of one another, are fluorine, chlorine, bromine, hydroxy, cyano, CF₃, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, amino, C₁₋₄-alkyl-amino and di-(C₁₋₄-alkyl)-amino, while the alkyl, cylcoalkyl groups may comprise one or more identical or different substituents selected from fluorine and hydroxy.

Particularly preferred substituents, independently of one another, are fluorine, chlorine, hydroxy, cyano, CF₃, hydroxymethyl, hydroxyethyl, C₁₋₃-alkyl, C₃₋₆-cycloalkyl, C₃₋₆-cycloalkyl-methyl, C₁₋₃-alkoxy-C₁₋₃-alkyl, C₁₋₃-alkoxy, amino, C₁₋₃-alkyl-amino and di-(C₁₋₃-alkyl)-amino.

According to this embodiment particularly preferred meanings of the group R¹ are prop-2-enyl, but-2-enyl, prop-2-ynyl, but-2-inyl, (1-hydroxy-C₃₋₆-cycloalkyl)-methyl, tetrahydropyran-3-yl, tetrahydropyran-4-yl, 2,3-dihydroxy-C₃₋₅-alkyl. Most particularly preferred meanings are prop-2-enyl, prop-2-ynyl, (1-hydroxy-cyclopropyl)methyl, tetrahydropyran-4-yl, 2,3-dihydroxypropyl, 2-hydroxy-1-(hydroxymethyl)-ethyl, 1,1-di(hydroxymethyl)-ethyl.

According to this first embodiment R² independently of R¹ has one of the meanings given hereinbefore for R¹ or R² has a meaning selected from the group consisting of H, C₁₋₈-alkyl, C₃₋₇-cycloalkyl or a phenyl or pyridinyl group optionally mono- or polysubstituted by the group R²⁰ and/or monosubstituted by nitro, while the alkyl or cycloalkyl group may be mono- or polysubstituted independently of one another by identical or different groups R¹¹, and a —CH₂— group in position 3 or 4 of a 5-, 6- or 7-membered cycloalkyl group may be replaced by —O—, —S— or —NR¹³—.

Preferred meanings of the group R¹¹ are F, Cl, Br, C₁₋₆-alkyl, C₂₋₆-alkenyl, C₂₋₆-alkynyl, R¹⁵—O— cyano, R¹⁶R¹⁷N, C₃₋₇-cycloalkyl, cyclo-C₃₋₆-alkyleneimino, pyrrolidinyl, N—(C₁₋₄-alkyl)-pyrrolidinyl, piperidinyl, N—(C₁₋₄-alkyl)-piperidinyl, phenyl and pyridyl, while in the above-mentioned groups and radicals one or more C atoms may independently of one another be mono- or polysubstituted by F, C₁₋₃-alkyl or hydroxy-C₁₋₃-alkyl, and/or one or two C atoms independently of one another may be monosubstituted by Cl, Br, OH, CF₃ or CN, and the above-mentioned cyclic groups may be mono- or polysubstituted at one or more C atoms by identical or different groups R²⁰, and in the case of a phenyl group may also additionally be monosubstituted by nitro, and/or one or more NH groups may be substituted by R²¹. If R¹¹ has one of the meanings R¹⁵—O—, cyano, R¹⁶R¹⁷N— or cyclo-C₃₋₆-alkyleneimino, the C atom of the alkyl or cycloalkyl group substituted by R¹¹ is not directly attached to a heteroatom, such as for example the group —N—X—.

Preferably the group R² denotes H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, phenyl, phenyl-C₁₋₃-alkyl, pyridyl or pyridyl-C₁₋₃-alkyl, while in the above-mentioned groups and radicals one or more C atoms may independently of one another be mono- or polysubstituted by F, C₁₋₃-alkyl or hydroxy-C₁₋₃-alkyl, and/or one or two C atoms may independently of one another be monosubstituted by Cl, Br, OH, CF₃ or CN, and the phenyl or pyridyl group may be mono- or polysubstituted by identical or different groups R²⁰ and/or monosubstituted by nitro. Preferred substituents of the above-mentioned phenyl or pyridyl groups are selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl- and di-(C₁₋₃-alkyl)-amino-C₁₋₃-alkyl, while a phenyl group may also be monosubstituted by nitro.

Particularly preferred meanings of the group R² are selected from the group consisting of H, C₁₋₄-alkyl, C₃₋₅-alkenyl, C₃₋₅-alkynyl, C₃₋₇-cycloalkyl, C₃₋₇cycloalkyl-C₁₋₃-alkyl, ω-(C₁₋₄-alkoxy)-C₂₋₃-alkyl, pyridyl and benzyl, while in the above-mentioned groups (with the exception of H) one or more C atoms may be mono- or polysubstituted independently of one another by F, C₁₋₃-alkyl or hydroxy-C₁₋₃-alkyl, and/or one or two C atoms may be monosubstituted independently of one another by Cl, Br, OH, CF₃ or CN.

Most particularly preferred groups R² are selected from among H, methyl, ethyl, n-propyl, i-propyl, prop-2-enyl, prop-2-ynyl, 2-methoxyethyl, cyclopentyl, cyclohexyl, cyclopropylmethyl, cyclopentylmethyl, (1-hydroxy-cyclopropyl)-methyl, 2-hydroxyethyl, 3-hydroxypropyl, benzyl and pyridyl.

According to a second embodiment the groups R¹, R² together with the N atom to which they are bound form a heterocyclic group which is selected from the meanings
dihydroxy-(cyclo-C₄₋₇-alkylene-imino),
(hydroxy-C₁₋₄-alkyl)-hydroxy-cyclo-C₃₋₇-alkylene-imino,
(hydroxy-C₁₋₃-alkyl)-cyclo-C₃₋₇-alkylene-imino, while in the last definition the C₁₋₃-alkyl group may be substituted by one or more identical or different C₁₋₃-alkyl groups, of which 2 alkyl groups may be joined together to form a C₃₋₇-cyloalkyl group;
   while the above-mentioned heterocyclic groups may be mono- or polysubstituted by substituents which are selected independently of one another from the group consisting of halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₂₋₄-alkenyl, C₂₋₄-alkynyl, amino, C₁₋₄-alkyl-amino and di-(C₁₋₄-alkyl)-amino, while alkyl, alkoxy or cycloalkyl groups contained in the above-mentioned substituents one or more C atoms may be mono- or polysubstituted by fluorine, and alkyl, alkoxy or cycloalkyl groups contained in the above-mentioned substituents one or more C atoms may be monosubstituted by chlorine, bromine or hydroxy.

According to this second embodiment preferred heterocyclic groups are 3,4-dihydroxypyrrolidinyl, 3,4-dihydroxypiperidinyl, 3,5-dihydroxypiperidinyl, (hydroxy-C₁₋₃-alkyl)-hydroxypyrrolidinyl, (hydroxy-C₁₋₃-alkyl)-hydroxy-piperidinyl, (hydroxy-C₃₋₆-cycloalkyl)-hydroxypyrrolidinyl, (hydroxy-C₃₋₆-cycloalkyl)-hydroxy-piperidinyl, (C₁₋₃-alkyl-hydroxy-methyl)-pyrrolidinyl, (C₁₋₃-alkyl-hydroxy-methyl)-piperidinyl, (di-C₁₋₃-alkyl-hydroxy-methyl)-pyrrolidinyl, (di-C₁₋₃-alkyl-hydroxy-methyl)-piperidinyl, (1-hydroxy-C₃₋₆-cycloalkyl)-pyrrolidinyl, (1-hydroxy-C₃₋₆-cycloalkyl)-piperidinyl, while the above-mentioned groups may be substituted as specified hereinbefore.

In the above-mentioned heterocyclic groups in one or two hydroxy groups the H atom may also be replaced by a methyl group.

Preferred substituents of the above-mentioned heterocyclic groups are fluorine, chlorine, hydroxy, CF₃, C₁₋₃-alkyl and hydroxy-C₁₋₃-alkyl, particularly methyl, ethyl and CF₃.

According to this second embodiment particularly preferred heterocyclic groups are 3,4-dihydroxypyrrolidinyl, 3,4-dihydroxypiperidinyl, 3,5-dihydroxypiperidinyl,(hydroxymethyl)-hydroxy-pyrrolidinyl, (hydroxymethyl)-hydroxy-piperidinyl, (1-hydroxyethyl)-hydroxy-pyrrolidinyl, (1-hydroxyethyl)-hydroxy-piperidinyl, (1-hydroxy-1-methylethyl)-hydroxy-pyrrolidinyl, (1-hydroxy-1-methylethyl)-hydroxy-piperidinyl, (1-hydroxycyclopropyl)-hydroxy-pyrrolidinyl, (1-hydroxycyclopropyl)-hydroxy-piperidinyl, (1-hydroxy-cyclopropyl)-pyrrolidinyl, (1-hydroxy-cyclopropyl)-piperidinyl, (1-hydroxyethyl)-pyrrolidinyl, (1-hydroxyethyl)-piperidinyl, (1-hydroxy-1-methylethyl)-pyrrolidinyl, (1-hydroxy-1-methylethyl)-piperidinyl, while the groups specified do not have any other substituents or have one or two substituents selected independently of one another from fluorine, hydroxy, C₁₋₃-alkyl, hydroxy-C₁₋₃-alkyl and CF₃.

Most particularly preferred definitions of the heterocyclic groups 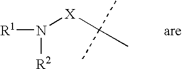 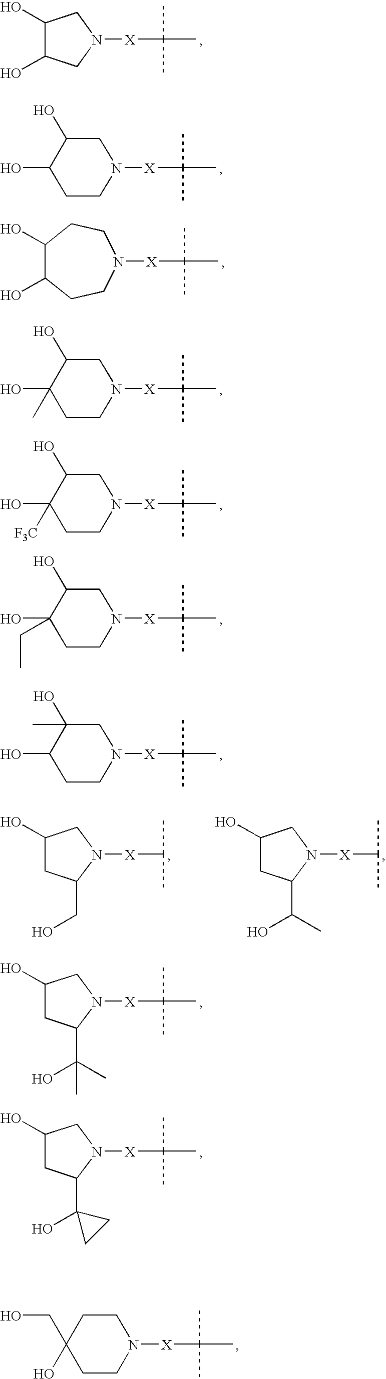 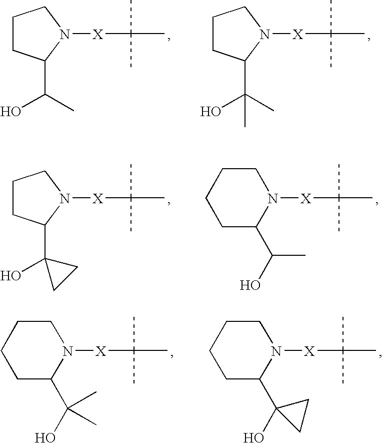
wherein X is defined as hereinbefore and particularly hereinafter, and
wherein the heterocyclic groups R¹R²N— specified are not further substituted, or
wherein methyl or ethyl groups may be mono-, di- or trisubstituted by fluorine, and wherein one or more H atoms of the heterocycle formed by the group R¹R²N— which are bound to carbon are substituted independently of one another by fluorine, chlorine, CN, CF₃, C₁₋₃-alkyl, hydroxy-C₁₋₃-alkyl, particularly C₁₋₃-alkyl or CF₃, preferably methyl, ethyl, CF₃.

According to a first embodiment the group X is preferably a C₂₋₄-alkylene bridge, particularly preferably ethylene or propylene, while one or two C atoms may be monosubstituted by hydroxy, hydroxy-C₁₋₃-alkyl or C₁₋₃-alkoxy, particularly hydroxy, and the alkylene bridge may be monosubstituted by C₂₋₆-alkenyl, C₂₋₆-alkynyl, C₃₋₆-cycloalkyl or C₃₋₆-cycloalkyl-C₁₋₃-alkyl and/or independently may be mono-, di- or trisubstituted by identical or different C₁₋₃-alkyl groups, and two alkyl groups may be joined together forming a 3- to 7-membered cycloalkyl group, or an alkyl group and an alkenyl group may be joined together forming a 5 to 7-membered cycloalkenyl group. In the group X one or more C atoms may be mono- or polysubstituted by F and/or Cl, preferably F.

According to a second embodiment the group X is preferably a C₃₋₄-alkylene bridge, wherein a —CH₂—CH₂— group not directly adjacent to the N atom of the R¹R²N— group is replaced by —CH═CH—, —C≡C—, —CH₂—O—, —CH₂—S— or —CH₂—NR⁴—, particularly preferably a —CH₂—CH═CH—, —CH₂—C≡C—, —CH₂—CH₂—O—, —CH₂—CH₂—S— or —CH₂—CH₂—NR⁴— bridge, while the meanings given for X hereinbefore may comprise a substituent selected from C₂₋₆-alkenyl, C₂₋₆-alkynyl, C₃₋₇-cycloalkyl and C₃₋₇-cycloalkyl-C₁₋₃-alkyl and/or one, two or three identical or different C₁₋₄-alkyl substituents, while two alkyl groups may be joined together forming a 3 to 7-membered cyclic group, or an alkyl and an alkenyl group may be joined together forming a 5 to 7-membered cyclic group. In the group X one or more C atoms may be mono- or polysubstituted by F and/or Cl, preferably F.

Particularly preferred meanings of X are therefore ethylene, propylene, —CH₂—CH═CH—, —CH₂—C≡C—, —CH₂—CH₂—O—, —CH₂—CH₂—S— or —CH₂—CH₂—NR⁴—.

A particularly preferred meaning of X is unsubstituted propylene or C₁₋₄-alkylene, particularly propylene, which comprises one or two identical or different substituents selected independently of one another from fluorine, chlorine, hydroxy and C₁₋₃-alkyl and/or a C₂₋₆-alkenyl or cyclopropyl substituent, while two alkyl substituents may be joined together forming a C₃₋₆-cycloalkyl group or an alkyl and an alkenyl group may be joined together forming a C₅₋₆-cycloalkenyl group. Particularly preferably, the alkylene bridge is mono- or disubstituted by identical or different groups selected from methyl, ethyl and i-propyl, while two alkyl groups may be joined together as specified, forming a cyclic group.

In the definition substituted propylene particularly preferred meanings are selected from the group consisting of

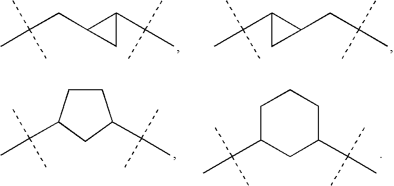

Other particularly preferred meanings for X are —CH₂—CH═CH—, —CH₂—C≡C—, —CH₂—CH₂—O—, —CH₂—CH₂—S— or —CH₂—CH₂—NR⁴—, particularly —CH₂—CH═CH—, —CH₂—CH₂—O— or —CH₂—CH₂—NR⁴, wherein R⁴ denotes H or C₁₋₄-alkyl, while the meanings specified for X are unsubstituted or comprise one or two identical or different substituents selected independently of one another from fluorine and C₁₋₃-alkyl and/or a cyclopropyl substituent, while two alkyl groups may be joined together forming a C₃₋₆-cycloalkyl group or if an alkyl group denotes the group R⁴, may be joined together forming a pyrrolidine or piperidine group. Particularly preferably, the meanings given for X hereinbefore may be mono- or disubstituted by identical or different groups selected from methyl, ethyl and i-propyl, while two alkyl groups as specified may be joined together forming a cyclic group.

A particularly preferred meaning of X is unsubstituted —CH₂—CH₂—O— or substituted —CH₂—CH₂—O— selected from the group consisting of

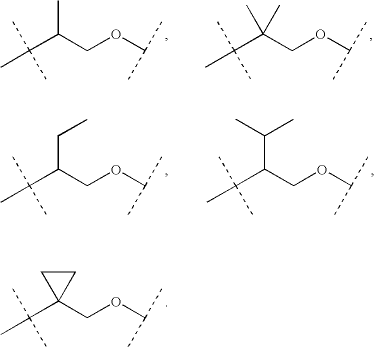

Another particularly preferred meaning of X is —CH₂—CH₂—NH— or —CH₂—CH₂—NCH₃— or substituted —CH₂—CH₂—NCH₃— selected from the group consisting of

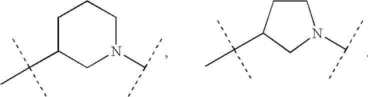

Another particularly preferred meaning of X is —CH₂—CH═CH— or substituted —CH₂—CH═CH— selected from the group consisting of

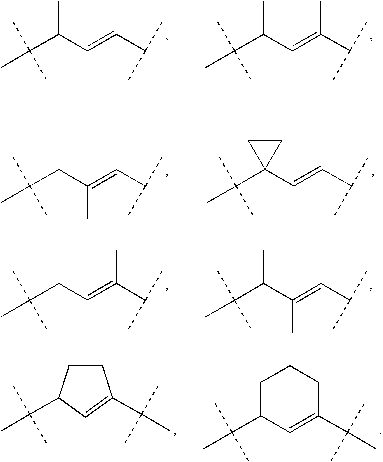

For X representing substituted alkenylene, only one of the two possible E/Z configurations is given above. Obviously, the other of the two E/Z configurations is also included according to the invention.

The position of the imino group within the alkylene bridge X is selected such that no aminal function is formed together with the amino group NR¹R² or another adjacent amino group, or two N atoms are not adjacent to one another.

Preferred meanings of the group R¹⁰ are —OH, methoxy and hydroxymethyl, particularly —OH.

According to a third embodiment the group X is a —CH₂— bridge, which is unsubstituted or substituted by one or two identical or different C₁₋₃-alkyl substituents and/or a substituent selected from C₂₋₆-alkenyl, C₂₋₆-alkynyl, C₃₋₆-cycloalkyl or C₃₋₆-cycloalkyl-C₁₋₃-alkyl, while two alkyl substituents may be joined together forming a 3- to 6-membered carbocyclic ring system. According to this embodiment X denoting —CH₂— is preferably unsubstituted or mono- or disubstituted by methyl, while two methyl substituents may be joined together forming a cyclopropyl group. This third embodiment regarding X is particularly preferred if Y is a bicyclic group, while the first ring of the bicyclic group is linked to X and the second ring is linked to Z.

The bridge W preferably denotes a single bond or ethylene, particularly preferably a single bond.

The bridge Z preferably denotes a single bond or ethylene which may comprise one or two methyl substituents, which may be joined together, forming a cyclopropyl group. Particularly preferably Z denotes a single bond.

The group Y preferably has a meaning selected from among the bivalent cyclic groups phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydro-isoquinolinyl, benzimidazolyl, benzoxazolyl, chromanyl, chromen-4-onyl, benzothienyl, or benzofuranyl, particularly preferably phenyl, pyridinyl, pyrimidinyl, pyrazinyl and pyridazinyl, while the above-mentioned cyclic groups may be mono- or polysubstituted at one or more C atoms by identical or different groups R²⁰, and in the case of a phenyl ring may also additionally be monosubstituted by nitro, and/or substituted by R²¹ at one or more N atoms.

If the group Y is a 6-membered cyclic or heterocyclic group, the bridges X and Z are preferably attached to the group Y in the para position.

Particularly preferably, the definition of the group Y is selected from among the bivalent cyclic groups 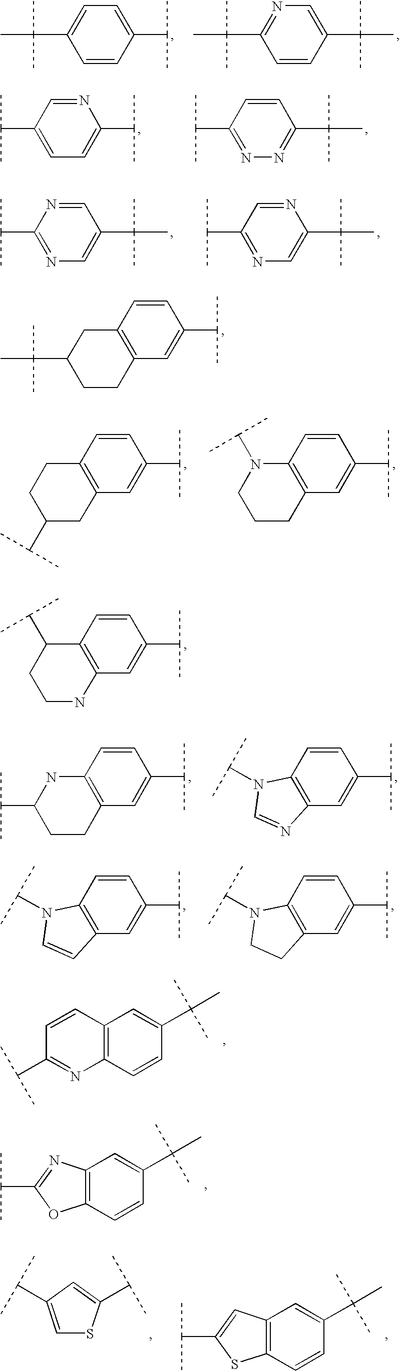 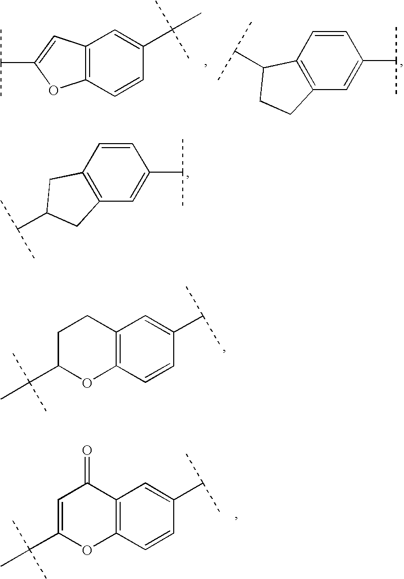
and particularly Y has one of the following meanings 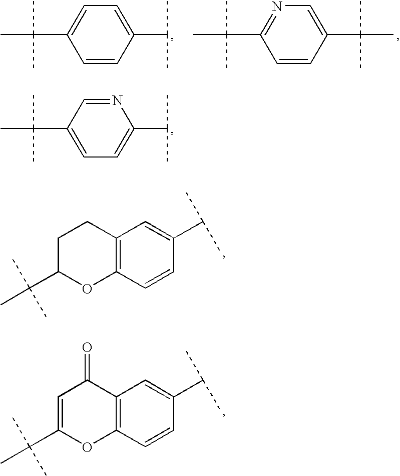
most particularly preferably, Y has one of the following meanings 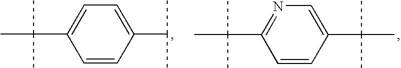
while the above-mentioned cyclic groups may be mono- or polysubstituted at one or more C atoms by identical or different groups R²⁰, and in the case of a phenyl ring may also additionally be monosubstituted by nitro, and/or one or more NH groups may be substituted by R²¹.

If the group Y is selected from among the bivalent bicyclic groups 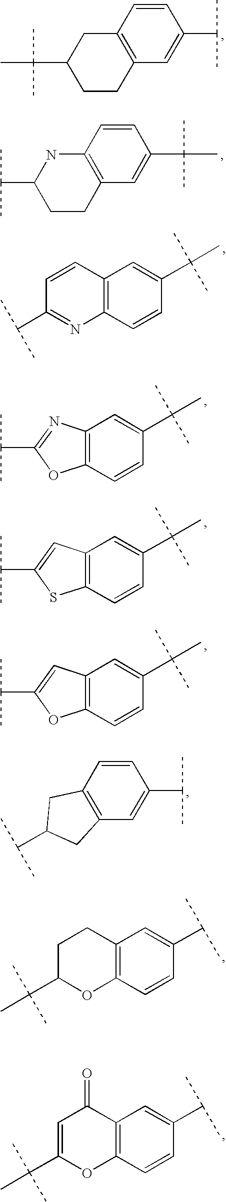
particularly if Y has one of the following meanings 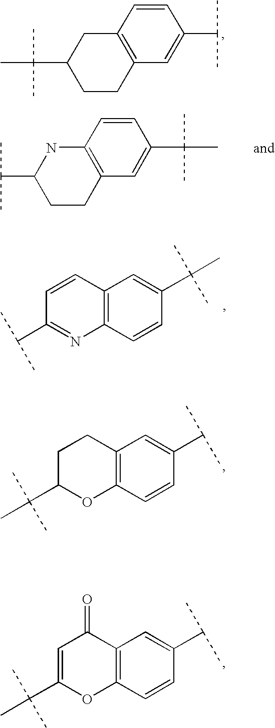
while the above-mentioned bicyclic groups may be mono- or polysubstituted at one or more C atoms by identical or different groups R²⁰, and in the case of a phenyl ring may also additionally be monosubstituted by nitro, and/or one or more NH groups may be substituted by R²¹,
the bridge X preferably denotes a —CH₂— group which may be substituted according to the third embodiment described hereinbefore in respect of X.

The group Y is preferably unsubstituted or mono- or disubstituted.

Particularly preferred substituents R²⁰ of the group Y are selected from the group consisting of fluorine, chlorine, bromine, cyano, nitro, C₁₋₄-alkyl, C₂₋₆-alkenyl, hydroxy, ω-hydroxy-C₁₋₃-alkyl, C₁₋₄-alkoxy, trifluoromethyl, trifluoromethoxy, C₂₋₄-alkynyl, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, aminocarbonyl, C₁₋₄-alkyl-amino-carbonyl- and di-(C₁₋₄-alkyl)-amino-carbonyl.

Most particularly preferred substituents R²⁰ of the group Y are selected from among fluorine, chlorine, bromine, cyano, C₁₋₃-alkyl, C₁₋₃-alkoxy, C₁₋₄-alkoxycarbonyl, trifluoromethyl, trifluoromethoxy, and in the case of a phenyl ring nitro as well.

Most particularly preferably the group Y denotes substituted phenylene of the partial formula 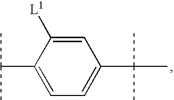
wherein L¹ has one of the meanings given hereinbefore for R²⁰, preferably F, Cl, Br, I, methyl, ethyl, ethenyl, ethynyl, CF₃, OCH₃, OCF₃, —CO—CH₃, —COOCH₃, CN or NO₂, or denotes H. Most particularly preferred meanings of the substituent L¹ are H, F, Cl, Br, methyl, ethyl, ethenyl, acetyl or methoxy.

Preferably the group A is selected from among the bivalent cyclic groups phenyl, pyridinyl, pyrimidinyl, pyrazinyl or pyridazinyl, which may be mono- or polysubstituted at one or more C atoms by identical or different groups R²⁰, and in the case of a phenyl ring may also additionally be monosubstituted by nitro.

Most particularly preferably, A is one of the groups listed hereinafter: 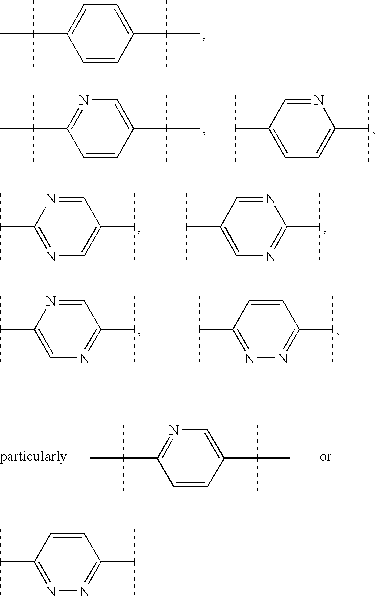
most particularly 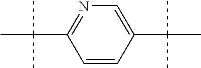
while the groups listed may be substituted as specified hereinbefore.

Particularly preferred substituents R²⁰ of the group A are, independently of one another, fluorine, chlorine, bromine, CF₃, amino, methoxy and C₁₋₃-alkyl.

Preferably the group A is unsubstituted or monosubstituted by R²⁰, as specified.

Preferred definitions of the group B according to a first preferred embodiment are selected from the group consisting of phenyl, pyridyl, thienyl and furanyl. Particularly preferably the group B denotes phenyl. The group B in the meanings specified may be mono- or polysubstituted by identical or different groups R²⁰, a phenyl group may additionally also be monosubstituted by nitro. Preferably the group B is unsubstituted or mono-, di- or trisubstituted, particularly unsubstituted or mono- or disubstituted. In the event of monosubstitution the substituent is preferably in the para position to the group A.

Preferred substituents R²⁰ of the group B are selected from the group consisting of fluorine, chlorine, bromine, cyano, nitro, C₁₋₄-alkyl, hydroxy, CHF₂, CHF₂—O—, hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-amino-carbonyl and di-(C₁₋₄-alkyl)-amino-carbonyl.

Particularly preferred substituents R²⁰ of the group B are selected from the group consisting of fluorine, chlorine, bromine, cyano, CF₃, C₁₋₃-alkyl, C₁₋₄-alkoxy and trifluoromethoxy.

Most particularly preferred substituents R²⁰ of the group B are selected from the group consisting of chlorine, bromine and methoxy.

According to a second embodiment the meaning of the group B is preferably selected from C₁₋₆-alkyl, C₂₋₆-alkenyl, C₂₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, C₃₋₇-cycloalkyl-C₁₋₃-alkynyl, while one or more C atoms in the groups mentioned hereinbefore for B may be mono- or polysubstituted by fluorine. In the cyclic groups according to the above-mentioned embodiment one or more C atoms may be substituted by identical or different R²⁰.

Particularly preferred according to this embodiment are the groups C₃₋₆-alkyl, C₃₋₆-alkenyl, C₃₋₆-alkynyl, cyclopentyl, cyclopentenyl, cyclohexyl, cyclohexenyl, cycloheptyl, cycloheptenyl, cyclopentyl-C₁₋₃-alkyl, cyclopentenyl-C₁₋₃-alkyl, cyclohexyl-C₁₋₃-alkyl, cyclohexenyl-C₁₋₃-alkyl, cycloheptyl-C₁₋₃-alkyl, cycloheptenyl-C₁₋₃-alkyl, while one or more C atoms in the groups mentioned hereinbefore for B may be mono- or polysubstituted by fluorine, and in cyclic groups one or more C atoms may be substituted by identical or different R²⁰.

Most particularly preferably according to this second embodiment, B denotes cyclohexenyl, which is unsubstituted or comprises 1, 2 or 3 identical or different substituents R²⁰, particularly methyl.

The following are preferred definitions of other substituents according to the invention:

R⁴ preferably denotes H, C₁₋₄-alkyl, C₃₋₆-cycloalkyl and C₃₋₆-cycloalkyl-methyl, particularly H, methyl, ethyl, propyl, i-propyl, n-propyl, cylcopropyl, cyclopentyl, cyclohexyl, cyclopropylmethyl, cyclopentylmethyl, cyclohexymethyl. Most particularly preferably R⁴ denotes H or methyl.

Preferably the substituent R¹³ has one of the meanings given for R¹⁶. Particularly preferably R¹³ denotes H, C₁₋₄-alkyl, C₃₋₇-cycloalkyl, C₃₋₇-cycloalkyl-C₁₋₃-alkyl, ω-hydroxy-C₂₋₃-alkyl, ω-(C₁₋₄-alkoxy)-C₂₋₃-alkyl-. Most particularly preferably R¹³ denotes H or C₁₋₄-alkyl. The alkyl groups specified hereinbefore may be monosubstituted by Cl or mono- or polysubstituted by F.

Preferred meanings of the substituent R¹⁵ are H, C₁₋₄-alkyl, C₃₋₇-cycloalkyl, C₃₋₇-cycloalkyl-C₁₋₃-alkyl, while, as hereinbefore defined, in each case one or more C atoms may additionally be mono- or polysubstituted by F and/or in each case one or two C atoms independently of one another may additionally be monosubstituted by Cl or Br. Particularly preferably R¹⁵ denotes H, CF₃, methyl, ethyl, propyl or butyl.

The substituent R¹⁶ preferably denotes H, C₁₋₄-alkyl, C₃₋₇-cycloalkyl, C₃₋₇-cycloalkyl-C₁₋₃-alkyl, ω-hydroxy-C₂₋₃-alkyl or ω-(C₁₋₄-alkoxy)-C₂₋₃-alkyl, while, as hereinbefore defined, in each case one or more C atoms may additionally be mono- or polysubstituted by F and/or in each case one or two C atoms independently of one another may additionally be monosubstituted by Cl or Br. Particularly preferably R¹⁶ denotes H, CF₃, C₁₋₃-alkyl, C₃₋₆-cycloalkyl or C₃₋₆-cycloalkyl-C₁₋₃-alkyl.

Preferably the substituent R¹⁷ has one of the meanings given for R¹⁶ which are specified as preferred or denotes phenyl, phenyl-C₁₋₃-alkyl, pyridinyl or C₁₋₄-alkylcarbonyl. Particularly preferably R¹⁷ has one of the meanings given for R¹⁶ which are specified as preferred.

Preferably one or both of the substituents R¹⁸ and R¹⁹ independently of one another denote hydrogen or C₁₋₄-alkyl, particularly hydrogen.

The substituent R²⁰ preferably denotes halogen, hydroxy, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy-C₁₋₄-alkyl, R²²—C₁₋₃-alkyl or has one of the meanings given for R²² which are specified as preferred, while, as hereinbefore defined, in each case one or more C atoms may additionally be mono- or polysubstituted by F and/or in each case one or two C atoms independently of one another may additionally be monosubstituted by Cl or Br.

Particularly preferred definitions of the group R²⁰ are halogen, hydroxy, cyano, C₁₋₄-alkyl, C₃₋₇-cycloalkyl, C₁₋₃-alkylcarbonyl and C₁₋₄-alkoxy, while, as hereinbefore defined, in each case one or more C atoms may additionally be mono- or polysubstituted by F and/or in each case one or two C atoms independently of one another may additionally be monosubstituted by Cl or Br. Most particularly preferably R²⁰ denotes F, Cl, Br, I, OH, cyano, methyl, difluoromethyl, trifluoromethyl, ethyl, n-propyl, acetyl, iso-propyl, methoxy, difluoromethoxy, trifluoromethoxy, ethoxy, n-propoxy or iso-propoxy.

The substituent R²² preferably denotes C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, C₁₋₄-alkyl-sulphonyl, C₁₋₄-alkyl-sulphinyl, C₁₋₄-alkyl-sulphonylamino, amino, C₁₋₄-alkylamino, di-(C₁₋₄-alkyl)-amino, C₁₋₄-alkyl-carbonyl-amino, hydroxy-C₁₋₃-alkylaminocarbonyl, aminocarbonylamino or C₁₋₄-alkylaminocarbonyl-amino, while, as hereinbefore defined, in each case one or more C atoms may additionally be mono- or polysubstituted by F and/or in each case one or two C atoms independently of one another may additionally be monosubstituted by Cl or Br. Most particularly preferred meanings of R²² are C₁₋₄-alkoxy, C₁₋₃-alkylcarbonyl, amino, C₁₋₄-alkylamino, di-(C₁₋₄-alkyl)-amino, wherein one or more H atoms may be replaced by fluorine.

Preferred definitions of the group R²¹ are C₁₋₄-alkyl, C₁₋₄-alkylcarbonyl, C₁₋₄-alkylsulphonyl, —SO₂—NH₂, —SO₂—NH—C₁₋₃-alkyl, —SO₂—N(C₁₋₃-alkyl)₂ and cyclo-C₃₋₆-alkyleneimino-sulphonyl, while, as hereinbefore defined, in each case one or more C atoms may additionally be mono- or polysubstituted by F and/or in each case one or two C atoms independently of one another may additionally be monosubstituted by Cl or Br. Most particularly preferably R²¹ denotes C₁₋₄-alkyl or CF₃.

Cy preferably denotes a C₃₋₇-cycloalkyl, particularly a C₃₋₆-cycloalkyl group, a C₅₋₇-cycloalkenyl group, pyrrolidinyl, piperidinyl, piperazinyl, morpholinyl, thiomorpholinyl, aryl or heteroaryl, and the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹. Most particularly preferred definitions of the group Cy are C₃₋₆-cycloalkyl, pyrrolidinyl and piperidinyl, which may be substituted as specified.

The term aryl preferably denotes phenyl or naphthyl, particularly phenyl.

The term heteroaryl preferably includes pyridyl, indolyl, quinolinyl and benzoxazolyl.

Preferred compounds according to the invention are those wherein one or more of the groups, radicals, substituents and/or indices have one of the meanings specified hereinbefore as being preferred.

Particularly preferred compounds according to the invention may be described by one of the general formulae IIa, IIb, IIc, IId, particularly IIa and IIb, 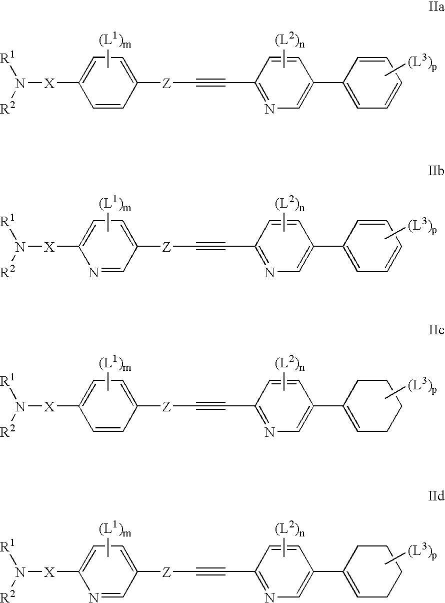
wherein
R¹, R², X and Z have one of the preferred meanings given above and

$$\begin{matrix}{L}^{1} , & {L}^{2} , \\ {L}^{3} , & \end{matrix}$$

independently of one another have one of the meanings given for R²⁰, and
m, n, p independently of one another represent the values 0, 1 or 2, and p may also denote 3.

In particular, in formulae IIa, IIb, IIc, IId,
Z denotes a single bond,
L¹ denotes fluorine, chlorine, bromine, cyano, C₁₋₃-alkyl, C₁₋₃-alkoxy, C₁₋₄-alkoxycarbonyl, trifluoromethyl, trifluoromethoxy, nitro,
m denotes 0 or 1,
L² denotes fluorine, chlorine, bromine, CN, amino, CF₃, methoxy and C₁₋₃-alkyl,
n denotes 0 or 1,
L³ independently of one another have a meaning selected from among fluorine, chlorine, bromine, cyano, nitro, C₁₋₄-alkyl, hydroxy, ω-hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-amino-carbonyl or di-(C₁₋₄-alkyl)-amino-carbonyl, particularly preferably fluorine, chlorine, bromine, cyano, CF₃, C₁₋₃-alkyl, C₁₋₄-alkoxy and trifluoromethoxy, with the proviso that a phenyl ring may only be monosubstituted by nitro, and
p denotes 0, 1, 2 or 3, particularly 1 or 2.

Most particularly preferably in formulae IIa, IIb, IIc, IId,
R¹ denotes prop-2-enyl, but-2-enyl, prop-2-ynyl, but-2-ynyl, (1-hydroxy-C₃₋₆-cycloalkyl)-methyl, tetrahydropyran-3-yl, tetrahydropyran-4-yl, 2,3-dihydroxy-C₃₋₅-alkyl, and
R² denotes H, C₁₋₄-alkyl, C₃₋₅-alkenyl, C₃₋₅-alkynyl, C₃₋₇-cycloalkyl, C₃₋₇-cycloalkyl-C₁₋₃-alkyl, ω-(C₁₋₄-alkoxy)-C₂₋₃-alkyl, pyridyl and benzyl, while in the above-mentioned groups (with the exception of H) one or more C atoms independently of one another may be mono- or polysubstituted by F, C₁₋₃-alkyl or hydroxy-C₁₋₃-alkyl, and/or one or two C atoms independently of one another may be monosubstituted by Cl, Br, OH, CF₃ or CN, or
R¹, R² are joined together and form, together with the N atom to which they are bound, a heterocyclic group which is selected from 3,4-dihydroxypyrrolidinyl, 3,4-dihydroxypiperidinyl, 3,5-dihydroxypiperidinyl,(hydroxymethyl)-hydroxy-pyrrolidinyl, (hydroxymethyl)-hydroxy-piperidinyl, (1-hydroxyethyl)-hydroxy-pyrrolidinyl, (1-hydroxyethyl)-hydroxy-piperidinyl, (1-hydroxy-1-methylethyl)-hydroxy-pyrrolidinyl, (1-hydroxy-1-methylethyl)-hydroxy-piperidinyl, (1-hydroxycyclopropyl)-hydroxy-pyrrolidinyl, (1-hydroxycyclopropyl)-hydroxy-piperidinyl, (1-hydroxy-cyclopropyl)-pyrrolidinyl, (1-hydroxy-cyclopropyl)-piperidinyl, (1-hydroxyethyl)-pyrrolidinyl, (1-hydroxyethyl)-piperidinyl, (1-hydroxy-1-methylethyl)-pyrrolidinyl, (1-hydroxy-1-methylethyl)-piperidinyl, while the specified groups have no other substituents or have one or two substituents selected independently of one another from fluorine, hydroxy, C₁₋₃-alkyl, hydroxy-C₁₋₃-alkyl, CF₃,
X denotes propylene, which is unsubstituted or has one or two identical or different substituents selected independently of one another from fluorine, chlorine, hydroxy and C₁₋₃-alkyl and/or a C₂₋₆-alkenyl or cyclopropyl substituent, while two alkyl substituents may be joined together, forming a C₃₋₆-cycloalkyl group, or an alkyl and an alkenyl group may be joined together, forming a C₅₋₆-cycloalkenyl group, or
—CH₂—CH═CH—, —CH₂—C≡C—, —CH₂—CH₂—O— or —CH₂—CH₂—NR⁴—-, which are unsubstituted or comprise one or two identical or different substituents selected independently of one another from fluorine and C₁₋₃-alkyl and/or a cyclopropyl substituent, while two alkyl groups may be joined together forming a C₃₋₆-cycloalkyl group or, if an alkyl group denotes the group R⁴, they may be joined together forming a pyrrolidine or piperidine group.

The compounds listed in the experimental section, including the tautomers, the diastereomers, the enantiomers, the mixtures thereof and the salts thereof, are preferred according to the invention.

Some expressions used hereinbefore and below to describe the compounds according to the invention will now be defined more fully.

The term halogen denotes an atom selected from among F, Cl, Br and I, particularly F, Cl and Br.

The term C₁₋ₙ-alkyl, where n has a value of 3 to 8, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methyl-ethylene (—CH(CH₃)—CH₂—), 1,1-dimethyl-ethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C-double bond. Examples of such groups include vinyl, 1-propenyl, 2-propenyl, iso-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl etc.

The term C₂₋ₙ-alkynyl, where n has a value of 3 to 6, denotes a branched or unbranched hydrocarbon group with 2 to n C atoms and a C≡C triple bond. Examples of such groups include ethynyl, 1-propynyl, 2-propynyl, iso-propynyl, 1-butynyl, 2-butynyl, 3-butynyl, 2-methyl-1-propynyl, 1-pentynyl, 2-pentynyl, 3-pentynyl, 4-pentynyl, 3-methyl-2-butynyl, 1-hexynyl, 2-hexynyl, 3-hexynyl, 4-hexynyl, 5-hexynyl etc.

The term C₁₋ₙ-alkoxy denotes a C₁₋ₙ-alkyl-O— group, wherein C₁₋ₙ-alkyl is defined as above. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylthio denotes a C₁₋ₙ-alkyl-S— group, wherein C₁₋ₙ-alkyl is defined as above. Examples of such groups include methylthio, ethylthio, n-propylthio, iso-propylthio, n-butylthio, iso-butylthio, sec-butylthio, tert-butylthio, n-pentylthio, iso-pentylthio, neo-pentylthio, tert-pentylthio, n-hexylthio, iso-hexylthio, etc.

The term C₁₋ₙ-alkylcarbonyl denotes a C₁₋ₙ-alkyl —C(═O)— group, wherein C₁₋ₙ-alkyl is defined as above. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic, preferably monocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3,2,1]octyl, spiro[4,5]decyl, norpinyl, norbonyl, norcaryl, adamantyl, etc.

The term C₅₋ₙ-cycloalkenyl denotes a monounsaturated mono-, bi-, tri- or spirocarbocyclic, preferably monocarboxylic group with 5 to n C atoms. Examples of such groups include cyclopentenyl, cyclohexenyl, cycloheptenyl, cyclooctenyl, cyclononenyl, etc.

The term C₃₋ₙ-cycloalkylcarbonyl denotes a C₃₋ₙ-cycloalkyl-C(═O) group, wherein C₃₋ₙ-cycloalkyl is as hereinbefore defined.

The term aryl denotes a carbocyclic, aromatic ring system, such as for example phenyl, biphenyl, naphthyl, anthracenyl, phenanthrenyl, fluorenyl, indenyl, pentalenyl, azulenyl, biphenylenyl, etc. A particularly preferred meaning of “aryl” is phenyl.

The term cyclo-C₃₋₆-alkyleneimino denotes a 4- to 7-membered ring which comprises 3 to 6 methylene units as well as an imino group, while the bond to the residue of the molecule is made via the imino group.

The term cyclo-C₃₋₆-alkyleneimino-carbonyl denotes a cyclo-C₃₋₆-alkyleneimino ring as hereinbefore defined which is linked to a carbonyl group via the imino group.

The term heteroaryl used in this application denotes a heterocyclic, aromatic ring system which comprises in addition to at least one C atom one or more heteroatoms selected from N, O and/or S. Examples of such groups are furanyl, thiophenyl, pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrolinyl, pyrazolinyl, indolinyl, oxazolidinyl, oxazolinyl, oxazepinyl, etc. Particularly preferably heteroaryl denotes a heteroaromatic mono- or bicyclic ring system.

Terms such as C₃₋₇-cycloalkyl-C₁₋ₙ-alkyl, heteroaryl-C₁₋ₙ-alkyl, etc. refer to C₁₋ₙ-alkyl, as defined above, which is substituted with a C₃₋₇-cycloalkyl, aryl or heteroaryl group.

Many of the terms given above may be used repeatedly in the definition of a formula or group and in each case have one of the meanings given above, independently of one another. Thus, for example, in the group di-C₁₋₄-alkyl-amino, the two alkyl groups may have the same or different meanings.

The term “unsaturated”, for example in “unsaturated carbocyclic group” or “unsaturated heterocyclic group”, as used particularly in the definition of the group Cy, comprises in addition to the mono- or polyunsaturated groups, the corresponding, totally unsaturated groups, but particularly the mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The style used hereinbefore and hereinafter, according to which in a cyclic group a bond of a substituent is shown towards the centre of this cyclic group, indicates unless otherwise stated that this substituent may be bound to any free position of the cyclic group carrying an H atom.

Thus in the example 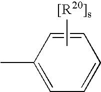
the substituent R²⁰ where s=1 may be bound to any of the free positions of the phenyl ring; where s=2 selected substituents R²⁰ may independently of one another be bound to different free positions of the phenyl ring.

The H atom of any carboxy group present or an H atom bound to an N atom (imino or amino group) may in each case be replaced by a group which can be cleaved in vivo. By a group which can be cleaved in vivo from an N atom is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or RₑCO—O—(R_{f}CR_{g})—O—CO— group wherein
Rₑ denotes a C₁₋₈-alkyl, C₅₋₇-cycloalkyl, phenyl or phenyl-C₁₋₃-alkyl group,
R_{f} denotes a hydrogen atom, a C₁₋₃-alkyl, C₅₋₇-cycloalkyl or phenyl group and
R_{g} denotes a hydrogen atom, a C₁₋₃-alkyl or RₑCO—O—(R_{f}CR_{g})—O— group wherein
Rₑ to R_{g} are as hereinbefore defined,
   while the phthalimido group is an additional possibility for an amino group, and the above-mentioned ester groups may also be used as a group which can be converted in vivo into a carboxy group.

The residues and substituents described above may be mono- or polysubstituted by fluorine as described. Preferred fluorinated alkyl groups are fluoromethyl, difluoromethyl and trifluoromethyl. Preferred fluorinated alkoxy groups are fluoromethoxy, difluoromethoxy and trifluoromethoxy. Preferred fluorinated alkylsulphinyl and alkylsulphonyl groups are trifluoromethylsulphinyl and trifluoromethylsulphonyl.

The compounds of general formula I according to the invention may have acid groups, predominantly carboxyl groups, and/or basic groups such as e.g. amino functions. Compounds of general formula I may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically useable bases such as alkali or alkaline earth metal hydroxides or carbonates, zinc or ammonium hydroxides or organic amines such as e.g. diethylamine, triethylamine, triethanolamine inter alia.

The compounds according to the invention may be obtained using methods of synthesis which are known in principle. Preferably the compounds are obtained analogously to the methods of preparation explained more fully hereinafter.

The two reaction plans A and B that follow show the synthesis of the compounds A.5 and B.5 according to the invention, while R¹, R², X, Y, Z, W, A and B have one of the meanings described hereinbefore. Hal denotes chlorine, bromine or iodine, particularly bromine or iodine, particularly preferably iodine.

According to reaction plan A the halogen compound A.1 is reacted with the alkyne compound A.2 in a molar ratio of about 1.5:1 to 1:1.5 under a protective has atmosphere in the presence of a suitable palladium catalyst, a suitable base and copper(I)iodide in a suitable solvent.

A preferred amount of copper(I)iodide is in the range from 1 to 15 mol %, particularly 5 to 10 mol % based on the educt A.1.

Suitable palladium catalysts are for example Pd(PPh₃)₄, Pd₂(dba)₃, Pd(OAc)₂, Pd(PPh₃)₂Cl₂, Pd(CH₃CN)₂Cl₂, Pd(dppf)Cl₂. The palladium catalyst is preferably used in an amount from 1 to 15 mol %, particularly 5 to 10 mol % based on the educt A.1.

Suitable bases are particularly amines, such as for example triethylamine or ethyldiisopropylamine, as well as Cs₂CO₃. The base is preferably used in an at least equimolar amount based on the educt A.1, in excess or as the solvent. Moreover, suitable solvents are dimethylformamide or ether, such as for example tetrahydrofuran, including the mixtures thereof. The reaction takes place over a period of about 2 to 24 hours in a temperature range of about 20 to 90° C.

The alkyne compound A.3 obtained is reacted directly or after prior purification with methanesulphonic acid chloride to form the methanesulphonate derivative A.4. The reaction conditions required are known to the skilled man as such. Advantageous solvents are halogenated hydrocarbons, such as for example dichloromethane. Suitable reaction temperatures are usually in the range from 0 to 30° C.

The reaction solution containing the methanesulphonate derivative A.4 or the purified methanesulphonate derivative A.4, dissolved in a suitable solvent, is reacted with an amine H—NR¹R² to yield the end product A.5 and then optionally purified. If the amine H—NR¹R² has another primary or secondary amino function, this is advantageously provided with a protective group beforehand, which can be cleaved again after the reaction has ended, using methods known from the literature. The product thus obtained may for example be converted into the salt form by reaction with a corresponding acid. A preferred molar ratio of the derivative A.4 to the amine compound is in the range from 1.5:1 to 1:1.5. Suitable solvents are dimethylformamide or ether, such as for example tetrahydrofuran, including the mixtures thereof.

The reaction to form the product A.5 is advantageously carried out in a temperature range from about 20 to 90° C.
Reaction Plan A:

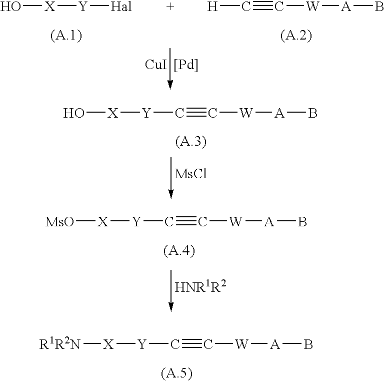

According to reaction plan B the halogen compound B.2 is reacted with the alkyne compound B.1 in a molar ratio of about 1.5:1 to 1:1.5 under a protective gas atmosphere in the presence of a suitable palladium catalyst, a suitable base and copper(I)iodide in a suitable solvent. Information on suitable reaction conditions, including catalysts, bases and solvents, may be found in the explanations of reaction plan A.

The alkyne compound B.3 obtained is reacted directly, or after prior purification, with methanesulphonic acid chloride to form the methanesulphonate derivative B.4. The reaction conditions to be respected can again be found in the remarks accompanying Diagram A.

The reaction solution containing the methanesulphonate derivative B.4 or the purified methanesulphonate derivative B.4, dissolved in a suitable solvent, is reacted with an amine H—NR¹R² to form the end product B.5 and then optionally purified. Here again, the remarks concerning Diagram A apply.
Reaction Plan B:

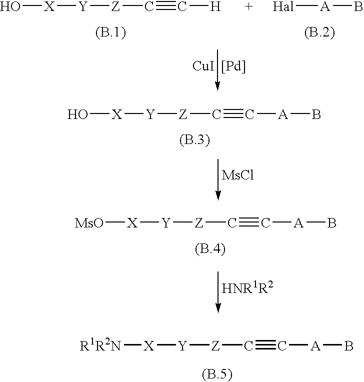

According to the other reaction plan C the halogen compound C.1 is reacted with the alkyne compound C.2 in a molar ratio of about 1.5:1 to 1:1.5 under a protective gas atmosphere in the presence of a suitable palladium catalyst, a suitable base and copper(I)iodide in a suitable solvent to form the product C.3 directly. Information on suitable reaction conditions, including catalysts, bases and solvents, may be found in the explanatory remarks accompanying reaction plan A.
Reaction Plan C:

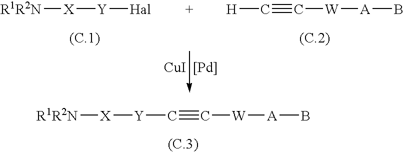

An alternative method of synthesis to this is shown in reaction plan D. According to this, the halogen compound D.2 is reacted with the alkyne compound D.1 in a molar ratio of about 1.5:1 to 1:1.5 under a protective gas atmosphere in the presence of a suitable palladium catalyst, a suitable base and copper(I)iodide in a suitable solvent to form the product D.3 directly. Once again, information on suitable reaction conditions, including catalysts, bases and solvents, may be found in the explanatory remarks accompanying reaction plan A.
Reaction Plan D:

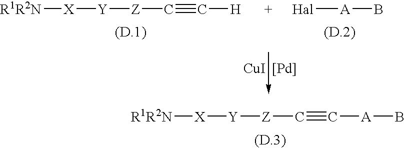

The reactions according to plans A, B, C and D are particularly advantageously carried out with the corresponding iodine compounds A.1, B.2, C.1 and D.2. In the event that Hal denotes bromine in compounds A.1, B.2, C.1 or D.2, it is advantageous to convert it into the corresponding iodine compound beforehand. One particularly advantageous method is the Aryl-Finkelstein reaction (Klapars, Artis; Buchwald, Stephen L. Copper-Catalyzed Halogen Exchange in Aryl Halides: An Aromatic Finkelstein Reaction. Journal of the American Chemical Society (2002), 124(50), 14844-14845). Thus, for example, the halogen compound A.1, B.2, C.1 or D.2 may be reacted with sodium iodide in the presence of N,N′-dimethyl-ethylenediamine and copper(I)iodide in a suitable solvent to form the corresponding iodine compound. An advantageous molar ratio of the halogen compound to sodium iodide is 1:1.8 to 1:2.3. N,N′-dimethyl-ethylenediamine is advantageously used in a molar ratio of 10 to 30 mol % based on the halogen compound A.1, B.2, C.1 or D.2. Preferred amounts of copper(I)iodide are in the range from 5 to 20 mol % based on the halogen compound A.1, B.2, C.1 or D.2. A suitable solvent is for example 1,4-dioxane. Suitable reaction temperatures are in the range from about 20 to 110° C. The reaction is substantially complete after 2 to 72 hours.

The compounds according to the invention may be obtained using methods of synthesis which are known in principle. Preferably the compounds are obtained analogously to the methods of preparation explained more fully in the experimental section.

Stereoisomeric compounds of formula (I) may chiefly be separated by conventional methods. The diastereomers are separated on the basis of their different physico-chemical properties, e.g. by fractional crystallisation from suitable solvents, by high pressure liquid or column chromatography, using chiral or preferably non-chiral stationary phases.

Racemates covered by general formula (I) may be separated for example by HPLC on suitable chiral stationary phases (e.g. Chiral AGP, Chiralpak AD). Racemates which contain a basic or acidic function can also be separated via the diastereomeric, optically active salts which are produced on reacting with an optically active acid, for example (+) or (−)-tartaric acid, (+) or (−)-diacetyl tartaric acid, (+) or (−)-monomethyl tartrate or (+)-camphorsulphonic acid, or an optically active base, for example with (R)-(+)-1-phenylethylamine, (S)-(−)-1-phenylethylamine or (S)-brucine.

According to a conventional method of separating isomers, the racemate of a compound of formula (I) is reacted with one of the above-mentioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example in a ratio by volume of 50:50, are used. Then each of the optically active salts is dissolved in water, carefully neutralised with a base such as sodium carbonate or potassium carbonate, or with a suitable acid, e.g. with dilute hydrochloric acid or aqueous methanesulphonic acid and in this way the corresponding free compound is obtained in the (+) or (−) form.

The (R) or (S) enantiomer alone or a mixture of two optically active diastereomeric compounds of general formula (I) may also be obtained by performing the syntheses described above with a suitable reaction component in the (R) or (S) configuration.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulphuric acid, phosphoric acid, methanesulphonic acid, ethanesulphonic acid, toluenesulphonic acid, benzenesulphonic acid, acetic acid, fumaric acid, succinic acid, lactic acid, citric acid, tartaric acid or maleic acid. Moreover, mixtures of the above mentioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly of sodium and potassium, are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have good intracerebral transitivity, particularly brain accessibility.

Therefore, MCH antagonists which contain at least one compound according to the invention are particularly suitable in mammals, such as for example rats, mice, guinea pigs, hares, dogs, cats, sheep, horses, pigs, cattle, monkeys and humans, for the treatment and/or prevention of symptoms and/or diseases which are caused by MCH or are otherwise causally connected with MCH.

Diseases caused by MCH or otherwise causally connected with MCH are particularly metabolic disorders, such as for example obesity, and eating disorders, such as for example bulimia, including bulimia nervosa. The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia and hyperphagia.

Compounds according to the invention may be particularly suitable for reducing hunger, curbing appetite, controlling eating behaviour and/or inducing a feeling of satiation.

In addition, the diseases caused by MCH or otherwise causally connected with MCH also include hyperlipidaemia, cellulitis, fatty accumulation, malignant mastocytosis, systemic mastocytosis, emotional disorders, affectivity disorders, depression, anxiety states, reproductive disorders, sexual disorders, memory disorders, epilepsy, forms of dementia and hormonal disorders.

Compounds according to the invention are also suitable as active substances for the prevention and/or treatment of other illnesses and/or disorders, particularly those which accompany obesity, such as for example diabetes, diabetes mellitus, particularly type II diabetes, hyperglycaemia, particularly chronic hyperglycaemia, complications of diabetes including diabetic retinopathy, diabetic neuropathy, diabetic nephropathy, etc., insulin resistance, pathological glucose tolerance, encephalorrhagia, cardiac insufficiency, cardiovascular diseases, particularly arteriosclerosis and high blood pressure, arthritis and gonitis.

MCH antagonists and formulations according to the invention may advantageously be used in combination with a dietary therapy, such as for example a dietary diabetes treatment, and exercise.

Another range of indications for which the compounds according to the invention are advantageously suitable is the prevention and/or treatment of micturition disorders, such as for example urinary incontinence, hyperactive bladder, urgency, nycturia, enuresis, while the hyperactive bladder and urgency may or may not be connected with benign prostatic hyperplasia.

The dosage required to achieve such an effect is conveniently, by intravenous or sub-cutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1 to 3× daily.

For this purpose, the compounds prepared according to the invention may be formulated, optionally in conjunction with other active substances as described hereinafter, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, lozenges, powders, granules, solutions, emulsions, syrups, aerosols for inhalation, ointments or suppositories.

In addition to pharmaceutical compositions the invention also includes compositions containing at least one alkyne compound according to the invention and/or a salt according to the invention optionally together with one or more physiologically acceptable excipients. Such compositions may also be for example foodstuffs which may be solid or liquid, in which the compound according to the invention is incorporated.

For the above mentioned combinations it is possible to use as additional active substances particularly those which for example potentiate the therapeutic effect of an MCH antagonist according to the invention in terms of one of the indications mentioned above and/or which make it possible to reduce the dosage of an MCH antagonist according to the invention. Preferably one or more additional active substances are selected from among
active substances for the treatment of diabetes,
active substances for the treatment of diabetic complications,
active substances for the treatment of obesity, preferably other than MCH antagonists,
active substances for the treatment of high blood pressure,
active substances for the treatment of hyperlipidaemia, including arteriosclerosis,
active substances for the treatment of arthritis,
active substances for the treatment of anxiety states,
active substances for the treatment of depression.

The above mentioned categories of active substances will now be explained in more detail by means of examples.

Examples of active substances for the treatment of diabetes are insulin sensitisers, insulin secretion accelerators, biguanides, insulins, α-glucosidase inhibitors, β3 adreno-receptor agonists.

Insulin sensitisers include pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702 and GW-1929.

Insulin secretion accelerators include sulphonylureas, such as for example tolbutamide, chloropropamide, tolazamide, acetohexamide, glyclopyramide and its ammonium salts, glibenclamide, gliclazide, glimepiride. Further examples of insulin secretion accelerators are repaglinide, nateglinide, mitiglinide (KAD-1229) and JTT-608.

Biguanides include metformin, buformin and phenformin.

Insulins include those obtained from animals, particularly cattle or pigs, semisynthetic human insulins which are synthesised enzymatically from insulin obtained from animals, human insulin obtained by genetic engineering, e.g. from Escherichi coli or yeasts. Moreover, the term insulin also includes insulin-zinc (containing 0.45 to 0.9 percent by weight of zinc) and protamine-insulin-zinc obtainable from zinc chloride, protamine sulphate and insulin. Insulin may also be obtained from insulin fragments or derivatives (for example INS-1, etc.).

Insulin may also include different kinds, e.g. with regard to the onset time and duration of effect (“ultra immediate action type”, “immediate action type”, “two phase type”, “intermediate type”, “prolonged action type”, etc.), which are selected depending on the pathological condition of the patient.

α-Glucosidase inhibitors include acarbose, voglibose, miglitol, emiglitate.

β₃ Adreno receptor agonists include AJ-9677, BMS-196085, SB-226552, AZ40140.

Active substances for the treatment of diabetes other than those mentioned above include ergoset, pramlintide, leptin, BAY-27-9955 as well as glycogen phosphorylase inhibitors, sorbitol dehydrogenase inhibitors, protein tyrosine phosphatase 1B inhibitors, dipeptidyl protease inhibitors, glipazide, glyburide.

Active substances for the treatment of diabetic complications include for example aldose reductase inhibitors, glycation inhibitors and protein kinase C inhibitors, DPPIV blockers, GLP-1 or GLP-2 analogues and SGLT-2 inhibitors.

Aldose reductase inhibitors are for example tolrestat, epalrestat, imirestat, zenarestat, SNK-860, zopolrestat, ARI-50i, AS-3201.

An example of a glycation inhibitor is pimagedine.

Protein Kinase C inhibitors are for example NGF, LY-333531.

DPPIV blockers are for example LAF237 (Novartis), MK431 (Merck) as well as 815541, 823093 and 825964 (all GlaxoSmithkline).

GLP-1 analogues are for example Liraglutide (NN2211) (NovoNordisk), CJC1131 (Conjuchem), Exenatide (Amylin).

SGLT-2 inhibitors are for example AVE-2268 (Aventis) and T-1095 (Tanabe, Johnson&Johnson).

Active substances other than those mentioned above for the treatment of diabetic complications include alprostadil, thiapride hydrochloride, cilostazol, mexiletine hydrochloride, ethyl eicosapentate, memantine, pimagedine (ALT-711).

Active substances for the treatment of obesity, preferably other than MCH antagonists, include lipase inhibitors and anorectics.

A preferred example of a lipase inhibitor is orlistat.

Examples of preferred anorectics are phentermine, mazindol, dexfenfluramine, fluoxetine, sibutramine, baiamine, (S)-sibutramine, SR-141716, NGD-95-1.

Active substances other than those mentioned above for the treatment of obesity include lipstatin.

Moreover, for the purposes of this application, the active substance group of anti-obesity active substances also includes the anorectics, of which the β₃ agonists, thyromimetic active substances and NPY antagonists should be emphasised. The range of substances which may be considered as preferred anti-obesity or anorectic active substances is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as for example sibutramine), a sympathomimetic active substance, a serotonergic active substance (such as for example dexfenfluramine, fenfluramine, a 5-HT2C agonist such as BVT.933), a dopamine antagonist (such as for example bromocriptine or pramipexol), a melanocyte-stimulating hormone receptor agonist or mimetic, an analogue of melanocyte-stimulating hormone, a cannabinoid receptor antagonist (Rimonabant, ACOMPLIA™), an MCH antagonist, the OB protein (hereinafter referred to as leptin), a leptin analogue, a leptin receptor agonist, a galanine antagonist, a GI lipase inhibitor or reducer (such as for example orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as for example exendin, and ciliary neurotrophic factors, such as for example axokines. In this context mention should also be made of the forms of therapy which produce weight loss by increasing the fatty acid oxidation in the peripheral tissue, such as for example inhibitors of acetyl-CoA carboxylase.

Active substances for the treatment of high blood pressure include inhibitors of angiotensin converting enzyme, calcium antagonists, potassium channel openers and angiotensin II antagonists.

Inhibitors of angiotensin converting enzyme include captopril, enalapril, alacepril, delapril (hydrochloride), lisinopril, imidapril, benazepril, cilazapril, temocapril, trandolapril, manidipine (hydrochloride).

Examples of calcium antagonists are nifedipine, amlodipine, efonidipine, nicardipine.

Potassium channel openers include levcromakalim, L-27152, AL0671, NIP-121.

Angiotensin II antagonists include telmisartan, losartan, candesartan cilexetil, valsartan, irbesartan, CS-866, E4177.

Active substances for the treatment of hyperlipidaemia, including arteriosclerosis, include HMG-CoA reductase inhibitors, fibrate compounds.

HMG-CoA reductase inhibitors include pravastatin, simvastatin, lovastatin, atorvastatin, fluvastatin, lipantil, cerivastatin, itavastatin, ZD-4522 and their salts.

Fibrate compounds include bezafibrate, clinofibrate, clofibrate and simfibrate.

Active substances for the treatment of arthritis include NSAIDs (non-steroidal antiinflammatory drugs), particularly COX2 inhibitors, such as for example meloxicam or ibuprofen.

Active substances for the treatment of anxiety states include chlordiazepoxide, diazepam, oxozolam, medazepam, cloxazolam, bromazepam, lorazepam, alprazolam, fludiazepam.

Active substances for the treatment of depression include fluoxetine, fluvoxamine, imipramine, paroxetine, sertraline.

The dosage for these active substances is conveniently ⅕ of the lowest normal recommended dose up to 1/1 of the normal recommended dose.

In another embodiment the invention also relates to the use of at least one alkyne compound according to the invention and/or a salt according to the invention for influencing the eating behaviour of a mammal. This use is particularly based on the fact that compounds according to the invention may be suitable for reducing hunger, curbing appetite, controlling eating behaviour and/or inducing a feeling of satiety. The eating behaviour is advantageously influenced so as to reduce food intake. Therefore, the compounds according to the invention are advantageously used for reducing body weight. Another use according to the invention is the prevention of increases in body weight, for example in people who had previously taken steps to lose weight and are interested in maintaining their lower body weight. According to this embodiment it is preferably a non-therapeutic use. Such a non-therapeutic use might be a cosmetic use, for example to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in metres) squared, is below a level of 30, particularly below 25.

The Examples that follow are intended to illustrate the invention:

Preliminary Remarks

As a rule, IR, ¹H-NMR and/or mass spectra have been obtained for the compounds prepared. Unless otherwise stated the R_{f} values were determined using ready-made silica gel 60 TLC plates F₂₅₄ (E. Merck, Darmstadt, Item no.1.05714) without chamber saturation. The R_{f} values obtained under the heading Alox were determined using ready-made aluminium oxide 60 TLC plates F₂₅₄ (E. Merck, Darmstadt, Item no. 1.05713) without chamber saturation.

For chromatographic purification, silica gel made by Messrs Millipore (MATREX™, 35-70 my) or Alox (E. Merck, Darmstadt, standardised aluminium oxide 90, 63-200 μm, Item No.: 1.01097.9050) is used. The ratios specified for the eluants are based on units by volume of the solvents in question.

The specified units by volume of NH₃ solutions relate to a concentrated solution of NH₃ in water. Unless otherwise stated the acid, base and salt solutions used for working up the reaction solutions are aqueous systems of the concentrations specified.

In the asymmetric dihydroxylations the “AD-Mix Alpha” (item number: 39,275-8) and “AD-Mix-Beta” (item number: 39,276-6) sold by Messrs Aldrich are used.

The HPLC data specified were measured under the parameters indicated below:

Analytical columns: Zorbax column (Agilent Technologies), SB (Stable Bond) C18; 3.5 μm; 4.6×75 mm; column temperature: 30° C.; flow: 0.8 mL/min; injection volume: 5 μL; detection at 254 nm (methods A, B and C).

Method A:

| | percent by volume | percent by volume |
|---|---|---|
| | of water | of acetonitrile |
| time (min) | (with 0.1% formic acid) | (with 0.1% formic acid) |
| 0 | 95 | 5 |
| 9 | 10 | 90 |
| 10 | 10 | 90 |
| 11 | 90 | 10 |

Method B:

| | percent by volume | percent by volume |
|---|---|---|
| | of water | of acetonitrile |
| time (min) | (with 0.1% formic acid) | (with 0.1% formic acid) |
| 0 | 95 | 5 |
| 4 | 10 | 90 |
| 10 | 10 | 90 |
| 11 | 90 | 10 |

Method C:

| | percent by volume | percent by volume |
|---|---|---|
| | of water | of acetonitrile |
| time (min) | (with 0.1% formic acid) | (with 0.1% formic acid) |
| 0 | 95 | 5 |
| 8 | 50 | 50 |
| 9 | 10 | 90 |
| 10 | 10 | 90 |
| 11 | 90 | 10 |

Preparative column: Zorbax column (Agilent Technologies), SB (Stable Bond)—C18; 3.5 μm; 30×100 mm; column temperature: ambient temperature; flow: 30 mL/min; detection at 254 nm.

In preparative HPLC purification, as a rule the same gradients are used which were used when obtaining the analytical HPLC data.

The products are collected under mass control, the fractions containing the product are combined and freeze-dried.

Temperatures are given in degrees Celsius (° C.); times are generally given in minutes (min), hours (h) or days (d). If there is no specific information as to the configuration, it is not clear whether there are pure enantiomers or whether partial or even total racemisation has taken place.

The following abbreviations are used above and hereinafter:
abs. absolute
CDI carbonyldiimidazole
Cyc cyclohexane
DCM dichloromethane
DIPE diisopropylether
DMF dimethylformamide
dppf 1,1′-bis(diphenylphosphino)ferrocene

EtOAc ethyl acetate
EtOH ethanol
i. vac. in vacuo
MeOH methanol
MTBE methyl-tert-butylether
PE petroleum ether
RT ambient temperature (approx. 20° C.)
TBAF tetrabutylammonium fluoride hydrate
THF tetrahydrofuran
dil. dilute
→* denotes the bonding site of a group
   Amine A1

### (3S,4R)-4-trifluoromethyl-piperidine-3,4-diol

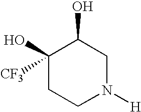

### A1a 1-benzyl-4-trifluoromethyl-pyridinium chloride

A solution of 7.59 mL (65.94 mmol) benzyl chloride in 10 mL acetonitrile is added to a solution of 10.0 g (65.94 mmol) 4-trifluoromethyl-pyridine in 40 mL acetonitrile and the mixture is stirred for 2 h at 80° C. Another 1.5 mL of benzyl chloride are added and the mixture is stirred for 22 h at 80° C. The reaction mixture is cooled to RT and combined with MTBE. The precipitate is filtered off, washed with MTBE, dried i. vac. and stored in the desiccator.

Yield: 14.48 g (80% of theoretical)

C₁₃H₁₁F₃N*Cl (M=273.681)

Calc.: molpeak (M+H)⁺: 238 Found: molpeak (M+H)⁺: 238

### A1b 1-benzyl-4-trifluoromethyl-1,2,3,6-tetrahydro-pyridine

3.0 g (79.36 mmol) NaBH₄ is added batchwise at 0° C., with vigorous cooling, to a solution of 14.48 g (52.91 mmol) 1-benzyl-4-trifluoromethyl-pyridinium chloride in 100 mL EtOH, then the cooling is removed and the reaction mixture is stirred for 1.5 h at 14° C. While cooling, 50 mL water and then 50 mL EtOH are added within 30 min. The reaction mixture is stirred for a further 30 min, the suspension formed is filtered and the filtrate is evaporated down i. vac.

Yield: 11.72 g (92% of theoretical)

C₁₃H₁₄F₃N (M=241.252)

Calc.: molpeak (M+H)⁺: 242 Found: molpeak (M+H)⁺: 242

HPLC-MS: 3.60 min (method B)

### A1c (3S,4R)-1-benzyl-4-trifluoromethyl-piperidine-3,4-diol

43.80 g AD-Mix-Beta are placed in 3 L tert-butanol/water (1:1) and stirred for 20 min at RT. The mixture is cooled to 0° C., 2.97 g (31.25 mmol) methanesulphonamide and 7.54 g (31.25 mmol) 1-benzyl-4-trifluoromethyl-1,2,3,6-tetrahydro-pyridine are added, the cooling bath is removed and the mixture is stirred for 8 d at RT. Another 22 g AD-Mix-Beta and 1.5 g methanesulphonamide are added and the mixture is again stirred for 7 d at RT. 11.2 g sodium sulphite are added and the mixture is stirred for 1 h. 200 mL semisaturated NaHCO₃ solution are added and the aqueous phase is exhaustively extracted with DCM. The combined organic phases are dried over Na₂SO₄ and evaporated down i. vac. The crude product is purified by MPLC-MS (Grom-Sil 120 ODS 4, 10 μm, gradient 0.15% formic acid in water/acetonitrile 90:10→10:90 in 10 min.). The eluates are combined, evaporated down i. vac. and neutralised with 100 mL semisaturated NaHCO₃ solution. The aqueous phase is extracted with EtOAc, the combined organic phases are dried over Na₂SO₄ and evaporated down i. vac.

Yield: 1.51 g (17% of theoretical)

C₁₃H₁₆F₃NO₂ (M=275.267)

Calc.: molpeak (M+H)⁺: 276 Found: molpeak (M+H)⁺: 276

R_{f} value: 0.40 (silica gel, Cyc/EtOAc 2:1)

### A1d (3S,4R)-4-trifluoromethyl-piperidine-3,4-diol

A mixture of 1.50 g (5.45 mmol) (3S,4R)-1-benzyl-4-trifluoromethyl-piperidine-3,4-diol and 170 mg Pd/C (10%) in 17 mL MeOH is hydrogenated at RT and 3 bar hydrogen pressure for 5 h. The catalyst is filtered off and the filtrate is evaporated down i. vac.

Yield: 910 mg (90% of theoretical)

C₆H₁₀F₃NO₂ (M=185.144)

Calc.: molpeak (M+H)⁺: 186 Found: molpeak (M+H)⁺: 186

R_{f} value: 0.35 (silica gel, EtOAc/MeOH/NH₃ 7:3:0.3)

Amine A2

### (3R,4S)-4-trifluoromethyl-piperidine-3,4-diol

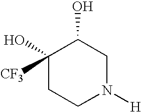

### A2a (3R,4S)-1-benzyl-4-trifluoromethyl-piperidine-3,4-diol

The product is obtained analogously to A1c starting from 5.8 g (24.04 mmol) 1-benzyl-4-trifluoromethyl-1,2,3,6-tetrahydro-pyridine and AD-Mix-Alpha. The crude product is purified by HPLC-MS.

Yield: 1.09 g (16% of theoretical)

C₁₃H₁₆F₃NO₂ (M=275.267)

Calc.: molpeak (M+H)⁺: 276 Found: molpeak (M+H)⁺: 276

HPLC-MS: 3.70 min (method A)

### A2b (3R,4S)-4-trifluoromethyl-piperidine-3,4-diol

The product is obtained analogously to A1d starting from 1.09 g (3.96 mmol) (3R,4S)-1-benzyl-4-trifluoromethyl-piperidine-3,4-diol.

Yield: 665 mg (91% of theoretical)

C₆H₁₀F₃NO₂ (M=185.144)

Calc.: molpeak (M+H)⁺: 186 Found: molpeak (M+H)⁺: 186

R_{f} value: 0.35 (silica gel, EtOAc/MeOH/NH₃ 7:3:0.3)

Amine A3

### (3R,4S)-4-methyl-piperidine-3,4-diol

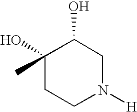

### A3a 1-benzyl-4-methyl-1,2,3,6-tetrahydro-pyridine

The product is obtained analogously to A1b starting from 10.0 g (45.5 mmol) 1-benzyl-4-methyl-pyridinium chloride.

Yield: 7.15 g (84% of theoretical)

C₁₃H₁₇N (M=187.281)

Calc.: molpeak (M+H)⁺: 188 Found: molpeak (M+H)⁺: 188

R_{f} value: 0.95 (silica gel, EtOAc/MeOH/NH₃ 9:1:0.1)

### A3b (3R,4S)-1-benzyl-4-methyl-piperidine-3,4-diol

Under a nitrogen atmosphere 14 g AD-Mix-Alpha in 50 mL water and 50 mL tert-butanol are taken and the mixture is stirred for 20 min at RT. Subsequently it is cooled to 0° C., 0.95 g (10.0 mmol) methanesulphonic acid amide and 1.87 g (10.0 mmol) 1-benzyl-4-methyl-1,2,3,6-tetrahydro-pyridine are added, the cooling bath is removed and the reaction mixture is stirred for 24 h at RT. 3.5 g sodium sulphite are added to the reaction mixture and it is stirred for 1 h. 200 mL DCM and 200 mL saturated NaHCO₃ solution are added, the organic phase is separated off and extracted with 100 mL KHSO₄ solution. The aqueous phase is made alkaline with saturated K₂CO₃ solution, extracted with 200 mL EtOAc and the organic phase is dried over Na₂SO₄. After the desiccant and solvent have been eliminated the crude product is purified by chromatography (silica gel, EtOAc/MeOH/NH₃ 19:1:0.1).

Yield: 1.23 g (56% of theoretical)

C₁₃H₁₉NO₂ (M=221.296)

Calc.: molpeak (M+H)⁺: 222 Found: molpeak (M+H)⁺: 222

R_{f} value: 0.56 (silica gel, EtOAc/MeOH/NH₃ 19:1:0.1)

### A3c (3R,4S)-4-methyl-piperidine-3,4-diol

The product is obtained analogously to A1d starting from 1.23 g (5.57 mmol) (3R,4S)-1-benzyl-4-methyl-piperidine-3,4-diol.

Yield: 730 mg (quant. yield)

C₆H₁₃NO₂ (M=131.173)

Calc.: molpeak (M+H)⁺: 132 Found: molpeak (M+H)⁺: 132

HPLC-MS: 0.93 min (method C)

Amine A4

### (3S,4R)-4-methyl-piperidine-3,4-diol

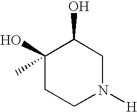

### A4a (3S,4R)-1-benzyl-4-methyl-piperidine-3,4-diol

The product is obtained analogously to A3b starting from 5.0 g (26.7 mmol) 1-benzyl-4-methyl-1,2,3,6-tetrahydro-pyridine and AD-Mix-Beta.

Yield: 4.68 g (79% of theoretical)

C₁₃H₁₉NO₂ (M=221.296)

Calc.: molpeak (M+H)⁺: 222 Found: molpeak (M+H)⁺: 222

R_{f} value: 0.54 (silica gel, EtOAc/MeOH/NH₃ 9:1:0.1)

### A4b (3S,4R)-4-methyl-piperidine-3,4-diol

The product is obtained analogously to A1d starting from 4.68 g (21.14 mmol) (3S,4R)-1-benzyl-4-methyl-piperidine-3,4-diol.

Yield: 2.33 g (84% of theoretical)

C₆H₁₃NO₂ (M=131.173)

Calc.: molpeak (M+H)⁺: 132 Found: molpeak (M+H)⁺: 132

R_{f} value: 0.05 (silica gel, EtOAc/MeOH/NH₃ 9:1:0.1)

Amine A5

### (3R,4S)-3-methyl-piperidine-3,4-diol

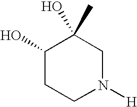

### A5a 1-benzyl-3-methyl-pyridinium chloride

The product is obtained analogously to A1a starting from 41.8 mL (430 mmol) of 3-methylpyridine.

Yield: 73.8 g (78% of theoretical)

C₁₃H₁₄N*Cl (M=219.710)

Calc.: molpeak (M)⁺: 184 Found: molpeak (M)⁺: 184

### A5b 1-benzyl-5-methyl-1,2,3,6-tetrahydro-pyridine

The product is obtained analogously to A1b starting from 40.0 g (182 mmol) 1-benzyl-3-methyl-pyridinium chloride.

Yield: 14.9 g (44% of theoretical)

C₁₃H₁₇N (M=187.281)

Calc.: molpeak (M+H)⁺: 188 Found: molpeak (M+H)⁺: 188

R_{f} value: 0.32 (silica gel, Cyc/EtOAc 4:1)

### A5c (3R,4S)-1-benzyl-3-methyl-piperidine-3,4-diol

The product is obtained analogously to A3b starting from 7.5 g (40.0 mmol) 1-benzyl-5-methyl-1,2,3,6-tetrahydro-pyridine and AD-Mix-Alpha.

Yield: 5.73 g (65% of theoretical)

C₁₃H₁₉NO₂ (M=221.296)

Calc.: molpeak (M+H)⁺: 222 Found: molpeak (M+H)⁺: 222

R_{f} value: 0.63 (silica gel, EtOAc/MeOH/NH₃ 9:1:0.1)

### A5d (3R,4S)-3-methyl-piperidine-3,4-diol

The product is obtained analogously to A1d starting from 5.73 g (25.9 mmol) (3R,4S)-1-benzyl-3-methyl-piperidine-3,4-diol.

Yield: 3.4 g (quant. yield)

C₆H₁₃NO₂ (M=131.173)

Calc.: molpeak (M+H)⁺: 132 Found: molpeak (M+H)⁺: 132

R_{f} value: 0.05 (silica gel, EtOAc/MeOH/NH₃ 9:1:0.1)

Amine A6

### (3S,4R)-3-methyl-piperidine-3,4-diol

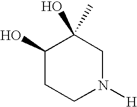

### A6a (3S,4R)-1-benzyl-3-methyl-piperidine-3,4-diol

The product is obtained analogously to A3b starting from 7.5 g (40.0 mmol) 1-benzyl-5-methyl-1,2,3,6-tetrahydro-pyridine and AD-Mix-Beta.

Yield: 7.42 g (84% of theoretical)

C₁₃H₁₉NO₂ (M=221.296)

Calc.: molpeak (M+H)⁺: 222 Found: molpeak (M+H)⁺: 222

R_{f} value: 0.63 (silica gel, EtOAc/MeOH/NH₃ 9:1:0.1)

### A6b (3S,4R)-3-methyl-piperidine-3,4-diol

The product is obtained analogously to A1d starting from 8.86 g (40.0 mmol) (3S,4R)-1-benzyl-3-methyl-piperidine-3,4-diol.

Yield: 5.03 g (96% of theoretical)

C₆H₁₃NO₂ (M=131.173)

Calc.: molpeak (M+H)⁺: 132 Found: molpeak (M+H)⁺: 132

R_{f} value: 0.17 (silica gel, EtOAc/MeOH/NH₃ 5:5:0.5)

Amine A7

### (3S,4R)-4-ethyl-piperidine-3,4-diol

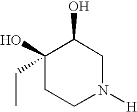

### A7a 1-benzyl-4-ethyl-pyridinium chloride

The product is obtained analogously to A1a starting from 100 mL (933 mmol) 4-ethylpyridine.

Yield: 143 g (66% of theoretical)

C₁₄H₁₆N*Cl (M=233.736)

Calc.: molpeak (M+H)⁺: 198 Found: molpeak (M+H)⁺: 198

R_{f} value: 0.12 (silica gel, EtOAc/MeOH/NH₃ 9:1:0.1)

### A7b 1-benzyl-4-ethyl-1,2,3,6-tetrahydro-pyridine

The product is obtained analogously to A1b starting from 143 g (614 mmol) 1-benzyl-4-ethyl-pyridinium chloride.

Yield: 99 g (80% of theoretical)

C₁₄H₁₉N (M=201.307)

Calc.: molpeak (M+H)⁺: 202 Found: molpeak (M+H)⁺: 202

R_{f} value: 0.91 (silica gel, EtOAc/MeOH/NH₃ 9:1:0.1)

### A7c (3S,4R)-1-benzyl-4-ethyl-piperidine-3,4-diol

The product is obtained analogously to A3b starting from 14.37 g (71.4 mmol) 1-benzyl-4-ethyl-1,2,3,6-tetrahydro-pyridine and AD-Mix-Beta.

Yield: 11.46 g (68% of theoretical)

C₁₄H₂₁NO₂ (M=235.322)

Calc.: molpeak (M+H)⁺: 236 Found: molpeak (M+H)⁺: 236

R_{f} value: 0.58 (silica gel, EtOAc/MeOH/NH₃ 95:5:0.5)

### A7d (3S,4R)-4-ethyl-piperidine-3,4-diol

The product may be obtained analogously to A1d starting from (3S,4R)-1-benzyl-4-ethyl-piperidine-3,4-diol.

The enantiomer (3R,4S)-4-ethyl-piperidine-3,4-diol may be obtained analogously to the sequence described.

Amine A8

### Cis-pyrrolidine-3,4-diol

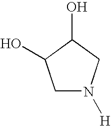

### A8a 1-benzyl-2,5-dihydro-1H-pyrrole

14.6 mL (127 mmol) benzyl chloride are added dropwise to a solution of 10 mL (127 mmol) 2,5-dihydro-1H-pyrrol in 100 mL acetonitrile, while the reaction mixture heats up to 45° C. After 1.5 h 300 mL MTBE is added to the suspension, the precipitate is suction filtered and the filtrate is evaporated down. The residue is purified by chromatography (silica gel, EtOAc/MeOH/NH₃ 19:1:0.1).

Yield: 4.0 g (20% of theoretical)

C₁₁H₁₃N (M=159.228)

Calc.: molpeak (M+H)⁺: 160 Found: molpeak (M+H)⁺: 160

R_{f} value: 0.60 (silica gel, EtOAc/MeOH/NH₃ 19:1:0.1)

### A8b cis-1-benzyl-pyrrolidine-3,4-diol

The product is obtained analogously to A3b starting from 4.0 g (25.1 mmol) 1-benzyl-2,5-dihydro-1H-pyrrol and AD-Mix-Beta.

Yield: 0.97 g (20% of theoretical)

C₁₁H₁₅NO₂ (M=193.242)

Calc.: molpeak (M+H)⁺: 194 Found: molpeak (M+H)⁺: 194

R_{f} value: 0.15 (silica gel, EtOAc/MeOH/NH₃ 9:1:0.1)

### A8c cis-pyrrolidine-3,4-diol

The product is obtained analogously to A1d starting from 0.97 g (5.04 mmol) cis-1-benzyl-pyrrolidine-3,4-diol.

Yield: 0.52 g (quant. yield)

C₄H₉NO₂ (M=103.120)

Calc.: molpeak (M+H)⁺: 104 Found: molpeak (M+H)⁺: 104

R_{f} value: 0.05 (silica gel, EtOAc/MeOH/NH₃ 5:5:0.5)

Amine A9

### (3R,4S)-piperidine-3,4-diol

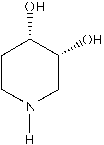

### A9a (3R,4S)-1-benzyl-piperidine-3,4-diol

The product is obtained analogously to A3b starting from 8.0 g (46.17 mmol) 1-benzyl-1,2,3,6-tetrahydro-pyridine and AD-Mix-Alpha.

Yield: 6.40 g (67% of theoretical)

C₁₂H₁₇NO₂ (M=207.269)

Calc.: molpeak (M+H)⁺: 208 Found: molpeak (M+H)⁺: 208

R_{f} value: 0.24 (silica gel, EtOAc/MeOH/NH₃ 9:1:0.1)

### A9b (3R,4S)-piperidine-3,4-diol

The product is obtained analogously to A1d starting from 6.4 g (30.86 mmol) (3R,4S)-1-benzyl-piperidine-3,4-diol.

Yield: 3.83 g (quant. yield)

C₅H₁₁NO₂ (M=117.146)

Calc.: molpeak (M+H)⁺: 118 Found: molpeak (M+H)⁺: 118

R_{f} value: 0.13 (silica gel, EtOAc/MeOH/NH₃ 5:5:0.5)

Amine A10

### (3S,4R)-piperidine-3,4-diol

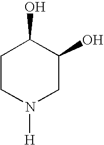

### A10a (3S,4R)-1-benzyl-piperidine-3,4-diol

The product is obtained analogously to A3b starting from 8.0 g (46.17 mmol) 1-benzyl-1,2,3,6-tetrahydro-pyridine and AD-Mix-Beta.

Yield: 6.13 g (64% of theoretical)

C₁₂H₁₇NO₂ (M=207.269)

Calc.: molpeak (M+H)⁺: 208 Found: molpeak (M+H)⁺: 208

R_{f} value: 0.35 (silica gel, EtOAc/MeOH/NH₃ 9:1:0.1)

### A10b (3S,4R)-piperidine-3,4-diol

The product is obtained analogously to A1d starting from 6.13 g (29.56 mmol) (3S,4R)-1-benzyl-piperidine-3,4-diol.

Yield: 3.75 g (quant. yield)

C₅H₁₁NO₂ (M=117.146)

Calc.: molpeak (M+H)⁺: 118 Found: molpeak (M+H)⁺: 118

R_{f} value: 0.12 (silica gel, EtOAc/MeOH/NH₃ 5:5:0.5)

Amine A11

### 4-hydroxymethyl-piperidin-4-ol

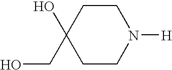

### A11a 1-benzyl-4-hydroxymethyl-piperidin-4-ol

The product is obtained analogously to A3b starting from 3.15 g (16.83 mmol) 1-benzyl-4-methylene-piperidine and AD-Mix-Alpha.

Yield: 2.92 g (79% of theoretical)

C₁₃H₁₉NO₂ (M=221.296)

Calc.: molpeak (M+H)⁺: 222 Found: molpeak (M+H)⁺: 222

R_{f} value: 0.12 (silica gel, EtOAc/MeOH/NH₃ 9:1:0.1)

### A11b 4-hydroxymethyl-piperidin-4-ol

The product is obtained analogously to A1d starting from 2.92 g (13.21 mmol) 1-benzyl-4-hydroxymethyl-piperidin-4-ol.

Yield: 1.88 g (quant. yield)

C₆H₁₃NO₂ (M=131.173)

Calc.: molpeak (M+H)⁺: 132 Found: molpeak (M+H)⁺: 132

R_{f} value: 0.06 (silica gel, EtOAc/MeOH/NH₃ 5:5:0.5)

Amine A12

### (S)-1-pyrrolidin-2-yl-cyclopropanol

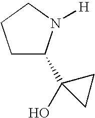

### A12a 1-((S)-1-benzyl-pyrrolidin-2-yl)-cyclopropanol

First 6.91 mL (23.57 mmol) titanium(IV)-isopropoxide and then 14.3 mL (42.9 mmol, 3 M in diethyl ether) ethylmagnesium bromide are slowly added dropwise to a solution of 5.0 g (21.43 mmol) N-benzyl-L-prolinethylester in 80 mL dry diethyl ether cooled to −15° C. and the reaction mixture is stirred for 30 min at this temperature. Subsequently 5.4 mL (42.9 mmol) boron trifluoride-diethyl ether complex are added at approx 10° C. and the mixture is stirred for a further 75 h at RT. While cooling the mixture is combined with 50 mL of 1 M NaOH, stirred for 1 h at RT, combined with 100 mL diethyl ether, the organic phase is separated off and dried over Na₂SO₄. After the desiccant and solvent have been eliminated the residue is purified by chromatography (silica gel, EtOAc).

Yield: 0.745 g (16% of theoretical)

C₁₄H₁₉NO (M=217.307)

Calc.: molpeak (M+H)⁺: 218 Found: molpeak (M+H)⁺: 218

R_{f} value: 0.17 (silica gel, EtOAc)

### A12b (S)-1-pyrrolidin-2-yl-cyclopropanol

The product is obtained analogously to A1d starting from 745 mg (3.43 mmol) 1-((S)-1-benzyl-pyrrolidin-2-yl)-cyclopropanol.

Yield: 350 mg (80% of theoretical)

C₇H₁₃NO (M=127.184)

Calc.: molpeak (M+H)⁺: 128 Found: molpeak (M+H)⁺: 128

R_{f} value: 0.10 (silica gel, EtOAc/MeOH/NH₃ 5:5:0.5)

Amine A13

### (2S,4R)-2-hydroxymethyl-pyrrolidine-4-ol

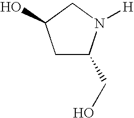

### A13a tert-butyl (2S,4R)-4-hydroxy-2-hydroxymethyl-pyrrolidine-1-carboxylate

54 mL (54.0 mmol) borane-THF complex are added dropwise to a solution, cooled to 0° C., of 5.0 g (21.62 mmol) 1-tert-butyl (2S,4R)-4-hydroxy-pyrrolidine-1,2-dicarboxylate in 25 mL dry THF, the reaction mixture is stirred for a further 15 min at 0° C. and then for 4 h at RT. While cooling with ice 60 mL MeOH are added, the mixture is stirred for a further 62 h at RT and then evaporated down i.vac. The residue obtained is purified by chromatography (silica gel, EtOAc/MeOH 19:1).

Yield: 4.53 g (96% of theoretical)

C₁₀H₁₉NO₄ (M=217.262)

Calc.: molpeak (M+H)⁺: 218 Found: molpeak (M+H)⁺: 218

R_{f} value: 0.50 (silica gel, EtOAc/MeOH 19:1)

### A13b (2S,4R)-2-hydroxymethyl-pyrrolidin-4-ol

16.0 mL TFA are added to a solution cooled to 0° C. of 4.53 g (20.85 mmol) tert-butyl (2S,4R)-4-hydroxy-2-hydroxymethyl-pyrrolidine-1-carboxylate in 200 mL DCM and the reaction mixture is stirred overnight at RT. It is evaporated down i. vac. and the product is freeze-dried. The product is obtained as the trifluoroacetate salt.

Yield: 4.73 g (96% of theoretical)

C₅H₁₁NO₂*C₂HF₃O₂ (M=231.170)

Calc.: molpeak (M+H)⁺: 118 Found: molpeak (M+H)⁺: 118

R_{f} value: 0.68 (silica gel, EtOAc/MeOH 9:1)

### EXAMPLE 1

### (3S,4R)-1-(2-{4-[5-(4-chloro-phenyl)-pyridin-2-ylethynyl]-2-methyl-phenoxy}-ethyl)-4-trifluoromethyl-piperidine-3,4-diol

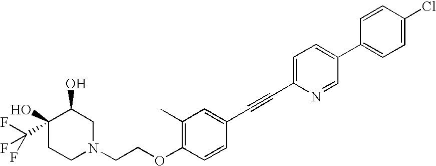

### 1a 2-(4-iodo-2-methyl-phenoxy)-ethanol

Under an N₂ atmosphere 2.34 g (10 mmol) 4-iodo-2-methyl-phenol are added batchwise to a suspension of 0.48 g (11 mmol) NaH in 50 mL THF cooled to 0° C. and tmis stirred for a further 30 min at this temperature. Then 0.85 mL (12 mmol) 2-bromoethanol, dissolved in 5 mL THF, are added dropwise and the mixture is stirred for 18 h at RT. 5 mL of DMF are added and the reaction mixture is heated to 70° C. for 8 h. It is evaporated down i. vac., the residue is taken up in water, extracted exhaustively with EtOAc and dried through Na₂SO₄. After the desiccant and solvent have been eliminated the residue is purified by chromatography (silica gel, Cyc/EtOAc 7:3).

Yield: 0.39 g (14% of theoretical)

C₉H₁₁IO₂ (M=278.091)

Calc.: molpeak (M+H)⁺: 279 Found: molpeak (M+H)⁺: 279

R_{f} value: 0.28 (silica gel, Cyc/EtOAc 2:1)

### 1b 2-(2-methyl-4-trimethylsilanylethynyl-phenoxy)-ethanol

31 mg (0.160 mmol) CuI are added under argon to a degassed solution of 2.23 g (8.00 mmol) 2-(4-iodo-2-methyl-phenoxy)-ethanol, 1.22 mL (8.80 mmol) trimethylsilylacetylene, 185 mg (0.160 mmol) tetrakis-triphenylphosphane-palladium and 2.38 mL (24.00 mmol) piperidine in 50 mL THF and the mixture is stirred for 1 h at RT. The reaction mixture is diluted with water and the aqueous phase is exhaustively extracted with EtOAc. The combined organic phases are washed with saturated NaCl solution and dried over Na₂SO₄. After the desiccant and solvent have been eliminated the residue is purified by chromatography (silica gel, Cyc/EtOAc 2:1).

Yield: 1.70 g (86% of theoretical)

C₁₄H₂₀O₂Si (M=248.393)

Calc.: molpeak (M+H)⁺: 249 Found: molpeak (M+H)⁺: 249

R_{f} value: 0.24 (silica gel, Cyc/EtOAc 2:1)

### 1c 2-(4-ethynyl-2-methyl-phenoxy)-ethanol

20.8 g (74.4 mmol) TBAF is added at RT to a solution of 16.8 g (67.6 mmol) 2-(2-methyl-4-trimethylsilanylethynyl-phenoxy)-ethanol in 500 mL THF and the mixture is stirred for 3 h at RT. The reaction mixture is evaporated down i. vac. and the residue dissolved in EtOAc. The organic phase is washed with water and saturated NaCl solution and dried over Na₂SO₄. After the desiccant and solvent have been eliminated the residue is reacted further without purification.

Yield: 12.0 g (quant. yield)

C₁₁H₁₂O₂ (M=176.212)

Calc.: molpeak (M)⁺: 176 Found: molpeak (M)⁺: 176

R_{f} value: 0.24 (silica gel, Cyc/EtOAc 2:1)

### 1d 2-[4-(5-bromo-pyridin-2-ylethynyl)-2-methyl-phenoxy]-ethanol

0.26 g (1.36 mmol) CuI are added to a degassed solution of 11.98 g (68.0 mmol) 2-(4-ethynyl-2-methyl-phenoxy)-ethanol, 16.11 g (68.0 mmol) 2,5-dibromopyridine, 0.96 g (1.36 mmol) bis-triphenylphosphane-palladium(II)-chloride and 19.22 mL (136.0 mmol) diisopropylamine in 500 mL THF and the mixture is stirred for 4 h at RT. The reaction mixture is evaporated down i. vac. and the residue is taken up in 800 mL EtOAc. The organic phase is washed with water and saturated NaCl solution and dried over Na₂SO₄. After the desiccant and solvent have been eliminated the residue is purified by chromatography (silica gel, gradient DCM/EtOAc 90:10→80:20).

Yield: 13.20 g (58% of theoretical)

C₁₆H₁₄BrNO₂ (M=332.192)

Calc.: molpeak (M+H)⁺: 332/334 (Br) Found: molpeak (M+H)⁺: 332/334 (Br)

R_{f} value: 0.39 (silica gel, Cyc/EtOAc 1:1)

### 1e 2-{4-[5-(4-chloro-phenyl)-pyridin-2-ylethynyl]-2-methyl-phenoxy}-ethanol

40 mL 2 N NaHCO₃ solution are added to a suspension of 13.20 g (39.74 mmol) 2-[4-(5-bromo-pyridin-2-ylethynyl)-2-methyl-phenoxy]-ethanol, 9.32 g (59.60 mmol) 4-chlorophenylboric acid and 2.30 g (1.99 mmol) tetrakis-triphenylphosphane-palladium in 400 mL 1,4-dioxane and the mixture is refluxed for 12 h. Another 4.66 g 4-chlorophenylboric acid and 1.14 g tetrakis-triphenylphosphane-palladium are added and the mixture is again refluxed for 8 h. The reaction mixture is evaporated down i. vac. and the residue is stirred with EtOAc and water. The precipitate is filtered off, washed with diethlyether and dried i. vac.

Yield: 10.70 g (74% of theoretical)

C₂₂H₁₈ClNO₂ (M=363.837)

Calc.: molpeak (M+H)⁺: 364/366 (Cl) Found: molpeak (M+H)⁺: 364/366 (Cl)

R_{f} value: 0.47 (silica gel, DCM/EtOAc 2:1)

### 1f 2-{4-[5-(4-chloro-phenyl)-pyridin-2-ylethynyl]-2-methyl-phenoxy}-ethyl methanesulphonate

2.74 mL (35.29 mmol) methanesulphonic acid chloride are added dropwise at 0° C. to a solution of 10.70 g (29.41 mmol) 2-{4-[5-(4-chloro-phenyl)-pyridin-2-ylethynyl]-2-methyl-phenoxy}-ethanol and 4.08 mL (29.41 mmol) triethylamine in 500 mL THF and the mixture is then stirred for 2 h at RT. The reaction mixture is filtered and the filtrate is evaporated down i. vac. The residue is stirred with diethyl ether and water, the precipitate is filtered off and dried i. vac.

Yield: 11.00 g (85% of theoretical)

C₂₃H₂₀ClNO₄S (M=441.928)

Calc.: molpeak (M+H)⁺: 442/444 (Cl) Found: molpeak (M+H)⁺: 442/444 (Cl)

R_{f} value: 0.73 (silica gel, DCM/EtOAc 1:1)

### 1g (3S,4R)-1-(2-{4-[5-(4-chloro-phenyl)-pyridin-2-ylethynyl]-2-methyl-phenoxy}-ethyl)-4-trifluoromethyl-piperidine-3,4-diol

A mixture of 82.6 mg (0.187 mmol) 2-{4-[5-(4-chloro-phenyl)-pyridin-2-ylethynyl]-2-methyl-phenoxy}-ethyl methanesulphonate, 69.2 mg (0.374 mmol) (3S,4R)-4-trifluoromethyl-piperidine-3,4-diol (amine A1) and 0.13 mL (0.748 mmol) N-ethyldiisopropylamine in 1.7 mL DMF is shaken for 40 h at 60° C. After filtration through an injection filter the crude product is purified by HPLC. The fractions containing the product are evaporated down i.vac., the residue is stirred with 20 mL EtOAc and 10 mL saturated NaHCO₃ solution, the organic phase is separated off and dried over Na₂SO₄. After the desiccant and solvent have been eliminated the residue is stirred with DIPE, suction filtered and dried.

Yield: 39.2 mg (39% of theoretical)

C₂₈H₂₆ClF₃N₂O₃ (M=530.966)

Calc.: molpeak (M+H)⁺: 531/533 (Cl) Found: molpeak (M+H)⁺: 531/533 (Cl)

HPLC-MS: 8.2 Min. (method A)

The following Examples are prepared analogously starting from 2-{4-[5-(4-chloro-phenyl)-pyridin-2-ylethynyl]-2-methyl-phenoxy}-ethyl methanesulphonate (Example 1f) and the corresponding amines:

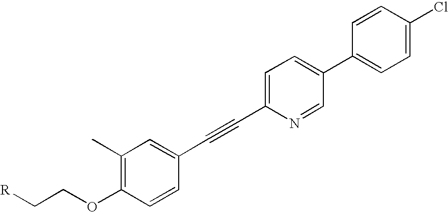

| | | | | | HPLC retention |
|---|---|---|---|---|---|
| | | | Empirical | Mass | time in min |
| Example | R | Yield (%) | formula | spectrum | (method) |
| 1.1 | 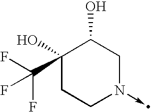 | 15 | C₂₈H₂₆CIF₃N₂O₃ | 531/533 [M + H]⁺ | 8.3 (A) |
| 1.2 | 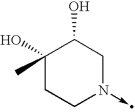 | 57 | C₂₈H₂₉CIN₂O₃ | 477/479 [M + H]⁺ | 7.6 (A) |
| 1.3 | 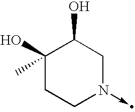 | 54 | C₂₈H₂₉CIN₂O₃ | 477/479 [M + H]⁺ | 7.5 (A) |
| 1.4 | 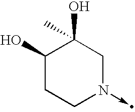 | 66 | C₂₈H₂₉CIN₂O₃ | 477/479 [M + H]⁺ | 7.6 (A) |
| 1.5 | 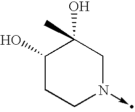 | 51 | C₂₈H₂₉CIN₂O₃ | 477/479 [M + H]⁺ | 7.6 (A) |
| 1.6 | 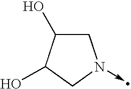 | 43 | C₂₆H₂₅CIN₂O₃ | 449/451 [M + H]⁺ | 7.5 (A) |
| 1.7 | 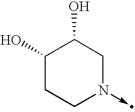 | 18 | C₂₇H₂₇CIN₂O₃ | 463/465 [M + H]⁺ | 7.4 (A) |
| 1.8 | 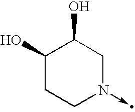 | 35 | C₂₇H₂₇CIN₂O₃ | 463/465 [M + H]⁺ | 7.4 (A) |
| 1.9 | 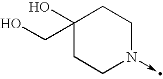 | 66 | C₂₈H₂₉CIN₂O₃ | 477/479 [M + H]⁺ | 7.4 (A) |
| 1.10 | 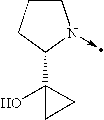 | 36 | C₂₉H₂₉CIN₂O₂ | 473/475 [M + H]⁺ | 0.58 (EtOAc/ MeOH/NH₃19:1:0.1) |
| 1.11 | 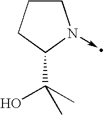 | 20 | C₂₉H₃₁CIN₂O₂ | 475/477 [M + H]⁺ | 0.20 (EtOAc/ MeOH/NH₃19:1:0.1) |
| 1.12 | 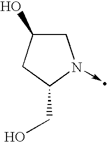 | 8 | C₂₇H₂₇CIN₂O₃ | 463/465 [M + H]⁺ | 7.5 (A) |
| 1.13 | 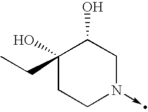 | | C₂₉H₃₁CIN₂O₃ | | |
| 1.14 | 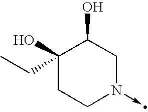 | | C₂₉H₃₁CIN₂O₃ | | |

### EXAMPLE 1.15

### (S)-3-[(2-{4-[5-(4-chloro-phenyl)-pyridin-2-ylethynyl]-2-methyl-phenoxy}-ethyl)-cyclopropylmethyl-amino]-propane-1,2-diol

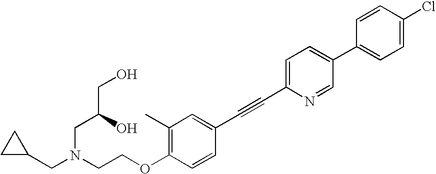

### 1.15a (2-{4-[5-(4-chloro-phenyl)-pyridin-2-ylethynyl]-2-methyl-phenoxy}-ethyl)-cyclopropylmethyl-amine

A mixture of 1.00 g (2.26 mmol) 2-{4-[5-(4-chloro-phenyl)-pyridin-2-ylethynyl]-2-methyl-phenoxy}-ethyl methanesulphonate, 0.99 mL (11.32 mmol) C-cyclopropyl-methylamine and 1.92 mL (11.32 mmol) N-ethyldiisopropylamine is stirred in 15 mL DMF for 72 h at 60° C. It is evaporated down i. vac. and the residue is purified by chromatography (silica gel, EtOAc/MeOH/NH₃ 19:1:0.1).

Yield: 0.27 g (29% of theoretical)

C₂₆H₂₅ClN₂O (M=416.942)

Calc.: molpeak (M+H)⁺: 417/419 (Cl) Found: molpeak (M+H)⁺: 417/419 (Cl)

R_{f} value: 0.39 (silica gel, DCM/MeOH/NH₃ 9:1:0.1)

### 1.15b (S)-3-[(2-{4-[5-(4-chloro-phenyl)-pyridin-2-ylethynyl]-2-methyl-phenoxy}-ethyl)-cyclopropylmethyl-amino]-propane-1,2-diol

A mixture of 70 mg (0.168 mmol) (2-{4-[5-(4-chloro-phenyl)-pyridin-2-ylethynyl]-2-methyl-phenoxy}-ethyl)-cyclopropylmethyl-amine, 56 μL (0.672 mmol) (R)-3-chloro-propane-1,2-diol and 0.12 mL (0.672 mmol) N-ethyldiisopropylamine in 1.7 mL DMF is shaken for 20 h at 80° C. Then another 100 μL (1.12 mmol) (R)-3-chloro-propane-1,2-diol are added and the reaction mixture is kept for a further 6 days at this temperature. It is evaporated down i. vac., the residue is distributed between 10 mL semisaturated NaHCO₃ solution and 20 mL EtOAc, the organic phase is separated off and dried over Na₂SO₄. After the desiccant and solvent have been eliminated the residue is purified by chromatography (silica gel, DCM/MeOH/NH₃ 98:2:0.2).

Yield: 10 mg (12% of theoretical)

C₂₉H₃₁ClN₂O₃ (M=491.021)

Calc.: molpeak (M+H)⁺: 491/493 (Cl) Found: molpeak (M+H)⁺: 491/493 (Cl)

R_{f} value: 0.30 (silica gel, DCM/MeOH/NH₃ 19:1:0.1)

### EXAMPLE 1.16

### (R)-3-[(2-{4-[5-(4-chloro-phenyl)-pyridin-2-ylethynyl]-2-methyl-phenoxy}-ethyl)-cyclopropylmethyl-amino]-propane-1,2-diol

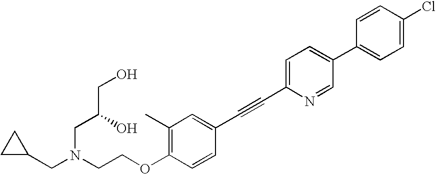

The product is prepared analogously to Example 1.15b starting from 70 mg (0.168 mmol) (2-{4-[5-(4-chloro-phenyl)-pyridin-2-ylethynyl]-2-methyl-phenoxy}-ethyl)-cyclopropylmethyl-amine and 0.28 mL (3.35 mmol) (S)-3-chloro-propane-1,2-diol.

Yield: 29.6 mg (36% of theoretical)

C₂₉H₃₁ClN₂O₃ (M=491.021)

Calc.: molpeak (M+H)⁺: 491/493 (Cl) Found: molpeak (M+H)⁺: 491/493 (Cl)

R_{f} value: 0.24 (silica gel, DCM/MeOH/NH₃ 19:1:0.1)

### EXAMPLE 1.17

### (2-{4-[5-(4-chloro-phenyl)-pyridin-2-ylethynyl]-2-methyl-phenoxy}-ethyl)-cyclopropylmethyl-prop-2-ynyl-amine

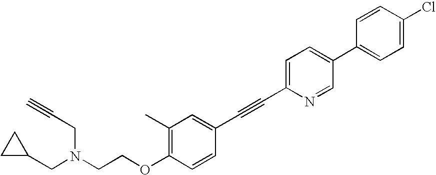

39.8 mg K₂CO₃ and 17 μL (0.16 mmol, 80% in toluene) 3-bromo-propyne are added to a solution of 60 mg (0.144 mmol) (2-{4-[5-(4-chloro-phenyl)-pyridin-2-ylethynyl]-2-methyl-phenoxy}-ethyl)-cyclopropylmethyl-amine in 2 mL DMF and the reaction mixture is stirred for 2 h at RT. It is evaporated down i. vac., the residue is distributed between 20 mL water and 40 mL EtOAc, the organic phase is separated off and dried over Na₂SO₄. After the desiccant and solvent have been eliminated the residue is purified by chromatography (Alox, Cyc/EtOAc 9:1).

Yield: 46.1 mg (70% of theoretical)

C₂₉H₂₇ClN₂O (M=454.990)

Calc.: molpeak (M+H)⁺: 455/457 (Cl) Found: molpeak (M+H)⁺: 455/457 (Cl)

R_{f} value: 0.90 (Alox, Cyc/EtOAc 2:1)

### EXAMPLE 1.18

### Allyl-(2-{4-[5-(4-chloro-phenyl)-pyridin-2-ylethynyl]-2-methyl-phenoxy}-ethyl)-cyclopentyl-amine

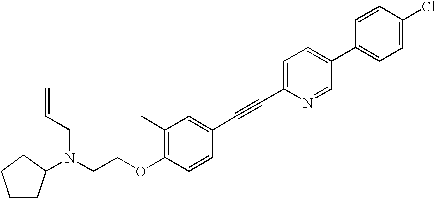

### 1.18a (2-{4-[5-(4-chloro-phenyl)-pyridin-2-ylethynyl]-2-methyl-phenoxy}-ethyl)-cyclopentyl-amine

A mixture of 1.33 g (3.0 mmol) 2-{4-[5-(4-chloro-phenyl)-pyridin-2-ylethynyl]-2-methyl-phenoxy}-ethyl methanesulphonate, 1.49 mL (15.0 mmol) cyclopentylamine and 2.57 mL (15.0 mmol) N-ethyldiisopropylamine in 30 mL DMF is stirred for 36 h at 60° C. It is evaporated down i. vac., the residue is stirred with water and EtOAc, suction filtered, the precipitate is dissolved in DCM, the organic phase is washed with semisaturated K₂CO₃ solution and the organic phase is dried over Na₂SO₄. After the desiccant and solvent have been eliminated the residue is stirred with diethyl ether, suction filtered and dried.

Yield: 0.82 g (63% of theoretical)

C₂₇H₂₇ClN₂O (M=430.969)

Calc.: molpeak (M+H)⁺: 431/433 (Cl) Found: molpeak (M+H)⁺: 431/433 (Cl)

R_{f} value: 0.21 (silica gel, DCM/MeOH 9:1)

### 1.18b allyl-(2-{4-[5-(4-chloro-phenyl)-pyridin-2-ylethynyl]-2-methyl-phenoxy}-ethyl)-cyclopentyl-amine

The product is prepared analogously to Example 1.17 starting from 86.2 mg (0.20 mmol) (2-{4-[5-(4-chloro-phenyl)-pyridin-2-ylethynyl]-2-methyl-phenoxy}-ethyl)-cyclopentyl-amine and 42 μL (0.48 mmol) 3-bromo-propene.

Yield: 5.1 mg (5% of theoretical)

C₃₀H₃₁ClN₂O (M=471.033)

Calc.: molpeak (M+H)⁺: 471/473 (Cl) Found: molpeak (M+H)⁺: 471/473 (Cl)

HPLC-MS: 6.4 min (method B)

### EXAMPLE 1.19

### Allyl-(2-{4-[5-(4-chloro-phenyl)-pyridin-2-ylethynyl]-2-methyl-phenoxy}-ethyl)-cyclopentylmethyl-amine

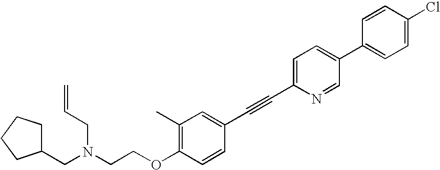

### 1.19a (2-{4-[5-(4-chloro-phenyl)-pyridin-2-ylethynyl]-2-methyl-phenoxy}-ethyl)-cyclopentylmethyl-amine

A mixture of 1.33 g (3.0 mmol) methanesulphonate 2-{4-[5-(4-chloro-phenyl)-pyridin-2-ylethynyl]-2-methyl-phenoxy}-ethyl, 595 mg (6.0 mmol) cyclopentyl-methylamine and 2.57 mL (15.0 mmol) N-ethyldiisopropylamine in 30 mL DMF is stirred for 20 h at 60° C. It is evaporated down i. vac., the residue is taken up in semiconcentrated K₂CO₃ solution, the organic phase is separated off and dried over Na₂SO₄. After the desiccant and solvent have been eliminated the residue is purified by chromatography (silica gel, DCM/MeOH 95:5).

Yield: 0.57 g (42% of theoretical)

C₂₈H₂₉ClN₂O (M=444.995)

Calc.: molpeak (M+H)⁺: 445/447 (Cl) Found: molpeak (M+H)⁺: 445/447 (Cl)

R_{f} value: 0.21 (silica gel, DCM/MeOH 95:5)

### 1.19b allyl-(2-{4-[5-(4-chloro-phenyl)-pyridin-2-ylethynyl]-2-methyl-phenoxy}-ethyl)-cyclopentylmethyl-amine

The product is prepared analogously to Example 1.17 starting from 89.0 mg (0.20 mmol) (2-{4-[5-(4-chloro-phenyl)-pyridin-2-ylethynyl]-2-methyl-phenoxy}-ethyl)-cyclopentylmethyl-amine and 21 μL (0.24 mmol) 3-bromo-propene.

Yield: 37.4 mg (39% of theoretical)

C₃₁H₃₃ClN₂O (M=485.059)

Calc.: molpeak (M+H)⁺: 485/487 (Cl) Found: molpeak (M+H)⁺: 485/487 (Cl)

HPLC-MS: 6.6 min (method B)

### EXAMPLE 1.20

### Allyl-(2-{4-[5-(4-chloro-phenyl)-pyridin-2-ylethynyl]-2-methyl-phenoxy}-ethyl)-amine

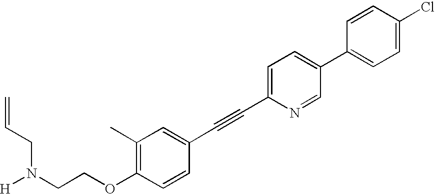

The product is prepared analogously to Example 1.19a starting from 400 mg (0.91 mmol) 2-{4-[5-(4-chloro-phenyl)-pyridin-2-ylethynyl]-2-methyl-phenoxy}-ethyl methanesulphonate and 0.2 mL (2.72 mmol) allylamine.

Yield: 228 mg (63% of theoretical)

C₂₅H₂₃ClN₂O (M=402.916)

Calc.: molpeak (M+H)⁺: 403/405 (Cl) Found: molpeak (M+H)⁺: 403/405 (Cl)

HPLC-MS: 5.4 min (method B)

### EXAMPLE 2

### (3S,4R)-1-(2-{4-[5-(4-chloro-phenyl)-pyridin-2-ylethynyl]-phenoxy}-ethyl)-4-trifluoromethyl-piperidine-3,4-diol

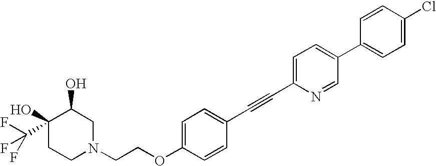

### 2a 2-(4-iodo-phenoxy)-ethanol

A suspension of 11 g (50 mmol) 4-iodophenol, 3.88 mL (55 mmol) 2-bromoethanol and 8.3 g (60 mmol) K₂CO₃ in 60 mL acetone is refluxed for 24 h. The solvent is removed i.vac., the residue is combined with water, exhaustively extracted with EtOAc and the organic phase is dried over Na₂SO₄. After the desiccant and solvent have been eliminated the residue is purified by chromatography (silica gel, Cyc/EtOAc 7:3).

Yield: 2.9 g (22% of theoretical)

C₈H₉IO₂ (M=264.064)

Calc.: molpeak (M)⁺: 264 Found: molpeak (M)⁺: 264

R_{f} value: 0.24 (silica gel, Cyc/EtOAc 2:1)

### 2b 2-{4-[5-(4-chloro-phenyl)-pyridin-2-ylethynyl]-phenoxy}-ethanol

Under an argon atmosphere 253 mg (0.22 mmol) tetrakis-triphenylphosphane-palladium and 42 mg (0.22 mmol) CuI are added to a solution of 2.9 g (11 mmol) 2-(4-iodo-phenoxy)-ethanol and 2.35 g (11 mmol) 5-(4-chloro-phenyl)-2-ethynyl-pyridine in 50 mL piperidine and the reaction mixture is stirred for 30 min at RT. The solvent is eliminated i.vac., the residue is combined with water and stirred with EtOAc. The precipitated product is suction filtered and dried.

Yield: 2.1 g (55% of theoretical)

C₂₁H₁₆ClNO₂ (M=349.820)

Calc.: molpeak (M+H)⁺: 350/352 (Cl) Found: molpeak (M+H)⁺: 350/352 (Cl)

R_{f} value: 0.42 (silica gel, Cyc/EtOAc 1:1)

### 2c 2-{4-[5-(4-chloro-phenyl)-pyridin-2-ylethynyl]-phenoxy}ethyl methanesulphonate

The product is obtained analogously to Example 1f starting from 4.40 g (12.58 mmol) 2-{4-[5-(4-chloro-phenyl)-pyridin-2-ylethynyl]-phenoxy}-ethanol.

Yield: 4.50 g (84% of theoretical)

C₂₂H₁₈ClNO₄S (M=427.901)

Calc.: molpeak (M+H)⁺: 428/430 (Cl) Found: molpeak (M+H)⁺: 428/430 (Cl)

R_{f} value: 0.88 (silica gel, EtOAc)

### 2d (3S,4R)-1-(2-{4-[5-(4-chloro-phenyl)-pyridin-2-ylethynyl]-phenoxy}-ethyl)-4-trifluoromethyl-piperidine-3,4-diol

The product is prepared analogously to Example 1g starting from 80 mg (0.187 mmol) 2-{4-[5-(4-chloro-phenyl)-pyridin-2-ylethynyl]-phenoxy}-ethyl methanesulphonate and 69.2 mg (0.374 mmol) (3S,4R)-4-trifluoromethyl-piperidine-3,4-diol (amine A1).

Yield: 43.6 mg (45% of theoretical)

C₂₇H₂₄ClF₃N₂O₃ (M=516.939)

Calc.: molpeak (M+H)⁺: 517/519 (Cl) Found: molpeak (M+H)⁺: 517/519 (Cl)

HPLC-MS: 7.7 min (method A)

The following Examples are prepared analogously starting from 2-{4-[5-(4-chloro-phenyl)-pyridin-2-ylethynyl]-phenoxy}-ethyl methanesulphonate (Example 2c):

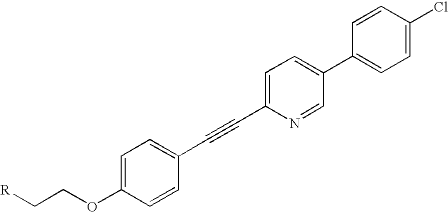

| | | | | | HPLC retention |
|---|---|---|---|---|---|
| | | | Empirical | Mass | time in min |
| Example | R | Yield (%) | formula | spectrum | (method) |
| 2.1 | 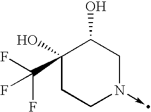 | 22 | C₂₇H₂₄CIF₃N₂O₃ | 517/519 [M + H]⁺ | 7.9 (A) |
| 2.2 | 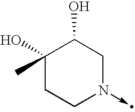 | 60 | C₂₇H₂₇CIN₂O₃ | 463/465 [M + H]⁺ | 7.2 (A) |
| 2.3 | 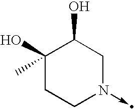 | 45 | C₂₇H₂₇CIN₂O₃ | 463/465 [M + H]⁺ | 7.2 (A) |
| 2.4 | 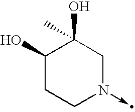 | 50 | C₂₇H₂₇CIN₂O₃ | 463/465 [M + H]⁺ | 7.3 (A) |
| 2.5 | 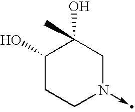 | 49 | C₂₇H₂₇CIN₂O₃ | 463/465 [M + H]⁺ | 7.3 (A) |
| 2.6 | 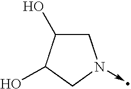 | 24 | C₂₅H₂₃CIN₂O₃ | 435/437 [M + H]⁺ | 7.2 (A) |
| 2.7 | 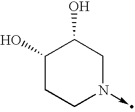 | 64 | C₂₆H₂₅CIN₂O₃ | 449/451 [M + H]⁺ | 6.5 (A) |
| 2.8 | 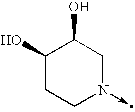 | 49 | C₂₆H₂₅CIN₂O₃ | 449/451 [M + H]⁺ | 6.6 (A) |
| 2.9 | 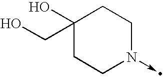 | 24 | C₂₇H₂₇CIN₂O₃ | 463/465 [M + H]⁺ | 7.1 (A) |
| 2.10 | 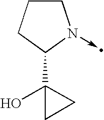 | 18 | C₂₈H₂₇CIN₂O₂ | 459/461 [M + H]⁺ | 8.0 (A) |
| 2.11 | 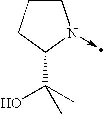 | 44 | C₂₈H₂₉CIN₂O₂ | 461/463 [M + H]⁺ | 5.3 (B) |
| 2.12 | 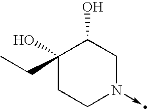 | | C₂₈H₂₉CIN₂O₃ | | |
| 2.13 | 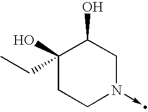 | | C₂₈H₂₉CIN₂O₃ | | |

### EXAMPLE 2.14

### (2-{4-[5-(4-chloro-phenyl)-pyridin-2-ylethynyl]-phenoxy}-ethyl)-cyclopropylmethyl-(tetrahydro-pyran-4-yl)-amine

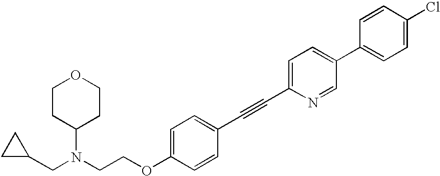

### 2.14a (2-{4-[5-(4-chloro-phenyl)-pyridin-2-ylethynyl]-phenoxy}-ethyl)-cyclopropylmethyl-amine

The product is prepared analogously to Example 1.19a starting from 1.71 g (4.0 mmol) 2-{4-[5-(4-chloro-phenyl)-pyridin-2-ylethynyl]-phenoxy}-ethyl methanesulphonate and 2.02 mL (20.0 mmol) C-cyclopropyl-methylamine.

Yield: 0.70 g (43% of theoretical)

C₂₅H₂₃ClN₂O (M=402.916)

Calc.: molpeak (M+H)⁺: 403/405 (Cl) Found: molpeak (M+H)⁺: 403/405 (Cl)

HPLC-MS: 5.1 min (method B)

### 2.14b (2-{4-[5-(4-chloro-phenyl)-pyridin-2-ylethynyl]-phenoxy}-ethyl)-cyclopropylmethyl-(tetrahydro-pyran-4-yl)-amine

37 μL (0.4 mmol) tetrahydro-pyran-4-on and 1 drop of glacial acetic acid are added to a solution of 80.6 mg (0.2 mmol) (2-{4-[5-(4-chloro-phenyl)-pyridin-2-ylethynyl]-phenoxy}-ethyl)-cyclopropylmethyl-amine in 10 mL THF and the reaction mixture is stirred for 15 min at RT, before the addition of 170 mg (0.8 mmol) NaBH(OAc)₃. The mixture is stirred for a further 16 h at RT, diluted with water, extracted exhaustively with EtOAc and dried over Na₂SO₄. After the desiccant and solvent have been eliminated the residue is purified by chromatography (silica gel, DCM/MeOH 95:5).

Yield: 9 mg (9% of theoretical)

C₃₀H₃₁ClN₂O₂ (M=487.032)

Calc.: molpeak (M+H)⁺: 487/489 (Cl) Found: molpeak (M+H)⁺: 487/489 (Cl)

R_{f} value: 0.52 (silica gel, DCM/MeOH 95:5)

### EXAMPLE 2.15

### 1-[(2-{4-[5-(4-chloro-phenyl)-pyridin-2-ylethynyl]-phenoxy}-ethylamino)-methyl]-cyclopropanol

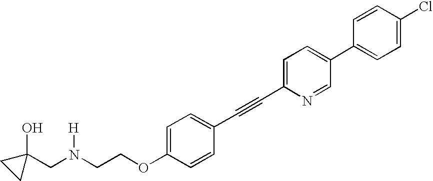

The product is prepared analogously to Example 1.19a starting from 1.71 g (4.0 mmol) 2-{4-[5-(4-chloro-phenyl)-pyridin-2-ylethynyl]-phenoxy}-ethyl methanesulphonate and 0.54 g (4.8 mmol) 1-aminomethyl-cyclopropanol.

Yield: 0.55 g (33% of theoretical)

C₂₅H₂₃ClN₂O₂ (M=418.915)

Calc.: molpeak (M+H)⁺: 419/421 (Cl) Found: molpeak (M+H)⁺: 419/421 (Cl)

R_{f} value: 0.18 (silica gel, EtOAc/MeOH/NH₃ 95:5:0.5)

HPLC-MS: 4.9 min (method B)

### EXAMPLE 2.16

### 1-{[(2-{4-[5-(4-chloro-phenyl)-pyridin-2-ylethynyl]-phenoxy}-ethyl)-(tetrahydro-pyran-4-yl)-amino]-methyl}-cyclopropanol

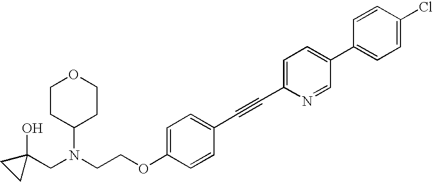

The product is prepared analogously to Example 2.14b starting from 83.8 mg (0.2 mmol) 1-[(2-{4-[5-(4-chloro-phenyl)-pyridin-2-ylethynyl]-phenoxy}-ethylamino)-methyl]-cyclopropanol and 37 μL (0.4 mmol) tetrahydro-pyran-4-one.

Yield: 2.1 mg (2% of theoretical)

C₃₀H₃₁ClN₂O₃ (M=503.032)

Calc.: molpeak (M+H)⁺: 503/505 (Cl) Found: molpeak (M+H)⁺: 503/505 (Cl)

R_{f} value: 0.47 (silica gel, DCM/MeOH 9:1)

The following Examples are prepared analogously starting from 1-[(2-{4-[5-(4-chloro-phenyl)-pyridin-2-ylethynyl]-phenoxy}-ethylamino)-methyl]-cyclopropanol (Example 2.15):

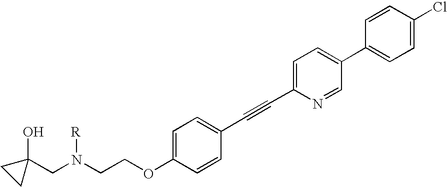

| | | | | | Rᵣ value |
|---|---|---|---|---|---|
| | | Yield | Empirical | Mass | on silica |
| Example | R | (%) | formula | spectrum | gel (eluant) |
| 2.17 | 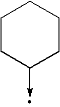 | 22 | C₃₁H₃₃CIN₂O₂ | 501/503 [M + H]⁺ | 0.70 (DCM/ MeOH 8:2) |
| 2.18 | 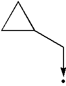 | 63 | C₂₉H₂₉CIN₂O₂ | 473/475 [M + H]⁺ | 0.84 (EtOAc/ MeOH/NH₃95:5:0.5) |
| 2.19 | 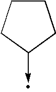 | 31 | C₃₀H₃₁CIN₂O₂ | 487/489 [M + H]⁺ | 0.57 (DCM/ MeOH 9:1) |

### EXAMPLE 2.20

### 1-{[(2-{4-[5-(4-chloro-phenyl)-pyridin-2-ylethynyl]-phenoxy}-ethyl)-(3-hydroxy-propyl)-amino]-methyl}-cyclopropanol

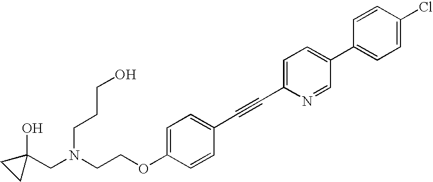

55.3 mg (0.4 mmol) K₂CO₃ and 36 μL (0.4 mmol) 3-bromo-1-propanol are added to a solution of 83.8 mg (0.2 mmol) 1-[(2-{4-[5-(4-chloro-phenyl)-pyridin-2-ylethynyl]-phenoxy}-ethylamino)-methyl]-cyclopropanol (Example 2.15) in 2 mL DMF and the reaction mixture is stirred for 24 h at RT. Another 36 μL (0.4 mmol) 3-bromo-1-propanol are added and the reaction mixture is heated to 50° C. for 8 h. It is evaporated down i. vac., the residue is taken up in water, extracted exhaustively with DCM and the combined organic phases are dried over Na₂SO₄. After the desiccant and solvent have been eliminated the residue is purified by chromatography (silica gel, EtOAc/MeOH/NH₃ 95:5:0.5).

Yield: 22 mg (23% of theoretical)

C₂₈H₂₉ClN₂O₃ (M=476.994)

Calc.: molpeak (M+H)⁺: 478/480 (Cl) Found: molpeak (M+H)⁺: 478/480 (Cl)

R_{f} value: 0.34 (silica gel, EtOAc/MeOH 4:1)

### EXAMPLE 2.21

### 1-{[(2-{4-[5-(4-chloro-phenyl)-pyridin-2-ylethynyl]-phenoxy}-ethyl)-propyl-amino]-methyl}-cyclopropanol

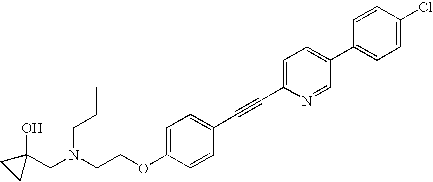

The product is prepared analogously to Example 2.20 starting from 83.8 mg (0.2 mmol) 1-[(2-{4-[5-(4-chloro-phenyl)-pyridin-2-ylethynyl]-phenoxy}-ethylamino)-methyl]-cyclopropanol (Example 2.15) and 23 μL (0.25 mmol) 1-bromo-propane.

Yield: 22 mg (24% of theoretical)

C₂₈H₂₉ClN₂O₂ (M=460.995)

Calc.: molpeak (M+H)⁺: 461/463 (Cl) Found: molpeak (M+H)⁺: 461/463 (Cl)

R_{f} value: 0.56 (silica gel, DCM/MeOH 95:5)

### EXAMPLE 3

### 2-((E)-3-{4-[5-(4-chloro-phenyl)-pyridin-2-ylethynyl]-phenyl}-allylamino)-propane-1,3-diol

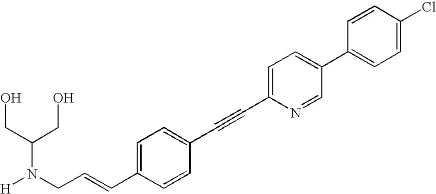

### 3a 5-bromo-2-[(tert-butyl-dimethyl-silanyl)-ethynyl]-pyridine

Under an argon atmosphere 0.80 g (4.20 mmol) CuI and 2.90 g (4.13 mmol) bis-triphenylphosphane-palladium(II)-chloride is added to a solution of 49.90 g (201.0 mmol) 2,5-dibromopyridine and 43.0 mL (225.6 mmol) tert-butyl-ethynyl-dimethyl-silane in 500 mL dry THF and 120 mL triethylamine at −7° C. and the mixture is stirred for 30 min at 0° C. The reaction mixture is stirred for a further 3.5 h at RT, then filtered and the filtrate is evaporated down i. vac. The residue is dissolved in 1 L EtOAc, the organic phase is washed with water and saturated NaCl solution, dried over Na2SO4 and evaporated down i. vac. The crude product is reacted further without purification.

Yield: 59.5 g (quant. yield)

C₁₃H₁₈BrNSi (M=296.278)

Calc.: molpeak (M+H)⁺: 296/298 (Br) Found: molpeak (M+H)⁺: 296/298 (Br)

R_{f} value: 0.75 (silica gel, Cyc/EtOAc 8:1)

### 3b 2-[(tert-butyl-dimethyl-silanyl)-ethynyl]-5-(4-chloro-phenyl)-pyridine

250 mL MeOH, 220 mL 2 N Na₂CO₃ solution and 1.80 g (2.46 mmol) PdCl₂(dppf) are added to a solution of 59.5 g (201.0 mmol) 5-bromo-2-[(tert-butyl-dimethyl-silanyl)-ethynyl]-pyridine and 36.5 g (233.4 mmol) 4-chlorophenylboric acid in 600 mL 1,4-dioxane and the mixture is refluxed for 1 h. The reaction mixture is evaporated down i. vac. and diluted with EtOAc. The organic phase is washed with water and semisaturated NaHCO₃ solution, dried over Na₂SO₄ and evaporated down i. vac. The residue is purified by column chromatography (silica gel, Cyc/EtOAc 9:1).

Yield: 38.5 g (58% of theoretical)

C₁₉H₂₂ClNSi (M=327.923)

Calc.: molpeak (M+H)⁺: 328/330 (Cl) Found: molpeak (M+H)⁺: 328/330 (Cl)

R_{f} value: 0.60 (silica gel, Cyc/EtOAc 8:1)

### 3c 5-(4-chloro-phenyl)-2-ethynyl-pyridine

43.66 g (156.0 mmol) TBAF are added at RT to a solution of 46.50 g (142.0 mmol) 2-[(tert-butyl-dimethyl-silanyl)-ethynyl]-5-(4-chloro-phenyl)-pyridine in 1 L DCM and the mixture is stirred for 2 h. The organic phase is washed with water, dried over Na₂SO₄ and evaporated down i. vac. The residue is stirred with DIPE, the precipitate is filtered off and washed with PE.

Yield: 26.0 g (86% of theoretical)

C₁₃H₈ClN (M=213.662)

Calc.: molpeak (M+H)⁺: 214/216 (Cl) Found: molpeak (M+H) :214/216 (Cl)

R_{f} value: 0.30 (silica gel, Cyc/EtOAc 4:1)

### 3d (E)-3-(4-iodo-phenyl)-prop-2-en-1-ol

Under a nitrogen atmosphere 12.0 g (80.0 mmol) NaI and 0.85 mL (8.0 mmol) N,N′-dimethylethylendiamine are added to a solution of 4.26 g (20.0 mmol) (E)-3-(4-bromo-phenyl)-prop-2-en-1-ol and 762 mg (4 mmol) CuI in 20 mL 1,4-dioxane and the reaction mixture is shaken for 17 h at 110° C. The reaction mixture is cooled to RT, combined with 200 mL EtOAc and 100 mL semiconcentrated NH₃ solution, vigorously stirred, the organic phase is separated off and dried over Na₂SO₄. After the desiccant and solvent have been eliminated the residue is reacted further without purification.

Yield: 4.69 g (90% of theoretical)

C₉H₉IO₂ (M=260.072)

Calc.: molpeak (M+H)⁺: 261 Found: molpeak (M+H)⁺: 261

HPLC-MS: 7.9 min (method A)

### 3e (E)-3-{4-[5-(4-chloro-phenyl)-pyridin-2-ylethynyl]-phenyl}-prop-2-en-1-ol

A solution of 3.12 g (12.0 mmol) (E)-3-(4-iodo-phenyl)-prop-2-en-1-ol, 3.33 g (15.0 mmol) 5-(4-chloro-phenyl)-2-ethynyl-pyridine and 4.31 mL (24 mmol) diisopropylamine in 120 mL dry THF is evacuated three times and then gassed with argon. Then 45 mg (0.24 mmol) CuI and 196 mg (0.24 mmol) PdCl₂(dppf) are added. The reaction mixture is stirred for 18 h at RT, the solvent is evaporated down i.vac., the residue is combined with 100 mL DCM and 50 mL semisaturated NaHCO₃ solution and vigorously stirred. The precipitate is separated off, washed with water and a little DCM, suspended in DIPE, suction filtered again and dried in the circulating air dryer at 50° C. until the weight remains constant.

Yield: 4.23 g (quant. yield)

C₂₂H₁₆ClNO (M=345.821)

Calc.: molpeak (M+H)⁺: 346/348 (Cl) Found: molpeak (M+H)⁺: 346/348 (Cl)

R_{f} value: 0.24 (silica gel, Cyc/EtOAc 2:1)

### 3f 5-(4-chloro-phenyl)-2-[4-((E)-3-chloro-propenyl)-phenylethynyl]-pyridine

A solution of 2.56 mL (35.28 mmol) thionyl chloride in 10 mL DCM is slowly added dropwise to a solution, cooled to 0° C., of 6.1 g (17.64 mmol) (E)-3-{4-[5-(4-chloro-phenyl)-pyridin-2-ylethynyl]-phenyl}-prop-2-en-1-ol in 80 ml DCM and the reaction solution is stirred for a further 2 h at 0° C. and 14 h at RT. It is again cooled to 0° C., 150 mL semisaturated NaHCO₃ solution are carefully added dropwise thereto, the organic phase is separated off and dried over Na₂SO₄. After the desiccant and solvent have been eliminated the residue is purified by chromatography (silica gel, Cyc/EtOAc 4:1).

Yield: 3.2 g (50% of theoretical)

C₂₂H₁₅Cl₂N (M=364.267)

Calc.: molpeak (M+H)⁺: 364/366/368 (2Cl) Found: molpeak (M+H)⁺: 364/366/368 (2CL)

R_{f} value: 0.60 (silica gel, Cyc/EtOAc 2:1)

### 3g 2-((E)-3-{4-[5-(4-chloro-phenyl)-pyridin-2-ylethynyl]-phenyl}-allylamino)-propane-1,3-diol

A solution of 70 mg (0.192 mmol) 5-(4-chloro-phenyl)-2-[4-((E)-3-chloro-propenyl)-phenylethynyl]-pyridine, 70 mg (0.768 mmol) 2-amino-1,3-propanediol and 0.13 mL (0.768 mmol) N-ethyldiisopropylamine in 1.7 mL DMF is shaken for 3.5 h at 60° C. The reaction mixture is filtered through an injection filter and purified directly by HPLC.

Yield: 37.6 mg (47% of theoretical)

C₂₅H₂₃ClN₂O₂ (M=418.915)

Calc.: molpeak (M+H)⁺: 419/421 (Cl) Found: molpeak (M+H)⁺: 419/421 (Cl)

HPLC-MS: 4.9 min (method B)

### EXAMPLE 3.1

### 2-((E)-3-{4-[5-(4-chloro-phenyl)-pyridin-2-ylethynyl]-phenyl}-allylamino)-2-methyl-propane-1,3-diol

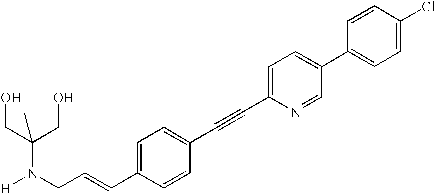

Prepared analogously to Example 3g from 70 mg (0.192 mmol) 5-(4-chloro-phenyl)-2-[4-((E)-3-chloro-propenyl)-phenylethynyl]-pyridine and 80.7 mg (0.768 mmol) 2-amino-2-methyl-1,3-propanediol.

Yield: 38.2 mg (46% of theoretical)

C₂₆H₂₅ClN₂O₂ (M=432.942)

Calc.: molpeak (M+H)⁺: 433/435 (Cl) Found: molpeak (M+H)⁺: 433/435 (Cl)

HPLC-MS: 4.9 min (method B)

The following compounds are prepared according to the process described in Example 3g:

| Example | Structure |
|---|---|
| 3.2 | 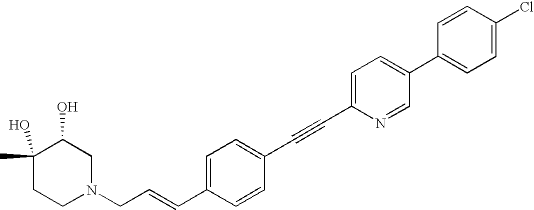 |
| 3.3 | 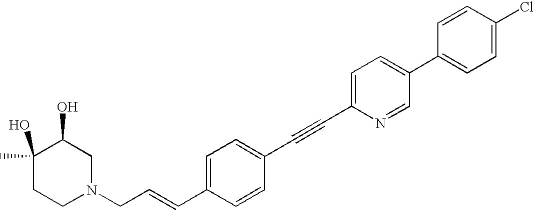 |
| 3.4 | 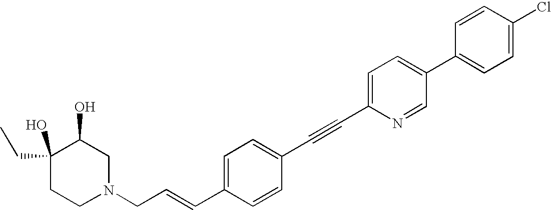 |
| 3.5 | 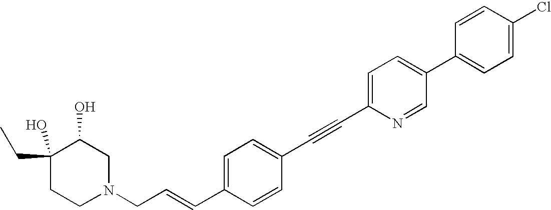 |
| 3.6 | 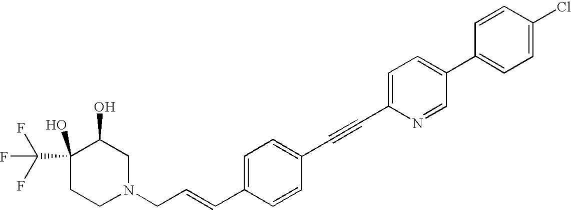 |
| 3.7 | 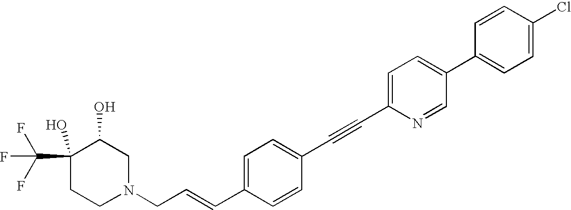 |

The following compounds are prepared starting from Example 3 according to the process described in Example 2.14b:

| Example | Structure |
|---|---|
| 3.8 | 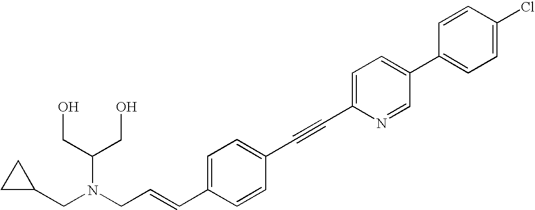 |
| 3.9 | 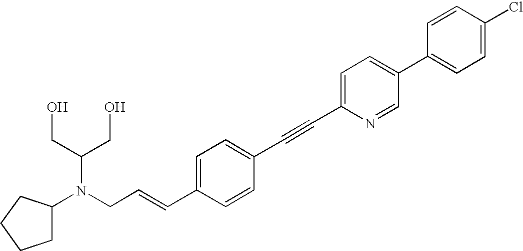 |

### EXAMPLE 4

### (3R,4S)-1-{5-[5-(4-chloro-phenyl)-pyridin-2-ylethynyl]-benzo[b]thiophen-2-ylmethyl}-4-methyl-piperidine-3,4-diol

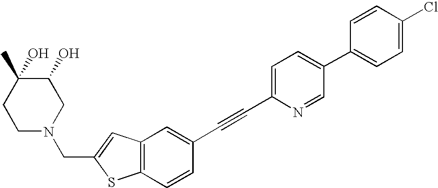

### 4a methyl 5-iodo-benzo[b]thiophene-2-carboxylate

Under an argon atmosphere 0.35 g (1.84 mmol) CuI, 5.53 g (36.88 mmol) NaI and 0.39 mL (3.67 mmol) N,N′-dimethylethylenediamine are added to a solution of 5.0 g (18.4 mmol) methyl 5-bromo-benzo[b]thiophene-2-carboxylate in 18 mL 1,4-dioxane and after flushing again with argon the reaction mixture is heated overnight to 110° C. After cooling the reaction mixture is combined with 30% NH₃ solution and water, exhaustively extracted with EtOAc, the combined organic phases are washed twice with water and dried over MgSO₄. After the desiccant and solvent have been eliminated the residue is triturated with DIPE and MTBE, suction filtered and dried in the air.

Yield: 3.4 g (58% of theoretical)

C₁₀H₇IO₂S (M=318.132)

Calc.: molpeak (M)⁺: 318 Found: molpeak (M)⁺: 318

HPLC-MS: 6.4 min (method B)

### 4b methyl 5-[5-(4-chloro-phenyl)-pyridin-2-ylethynyl]-benzo[b]thiophene-2-carboxylate

Under an argon atmosphere 3.7 mL (26.72 mmol) triethylamine are added to a solution of 3.4 g (10.69 mmol) methyl 5-iodo-benzo[b]thiophene-2-carboxylate and 2.48 g (10.69 mmol) 5-(4-chloro-phenyl)-2-ethynyl-pyridine in 20 mL THF and the reaction mixture is evacuated three times and in each case gassed with argon. Then 195 mg (0.267 mmol) PdCl₂(dppf)*DCM complex and 51 mg (0.267 mmol) CuI are added and the reaction mixture is stirred for 70 h at RT. EtOAc is added, the precipitated product is suction filtered, washed with a little EtOAc and dried in the air.

Yield: 3.0 g (70% of theoretical)

C₂₃H₁₄ClNO₂S (M=403.881)

Calc.: molpeak (M+H)⁺: 404/406 (Cl) Found: molpeak (M+H)⁺: 404/406 (Cl)

HPLC-MS: 7.3 min (method B)

### 4c 5-[5-(4-chloro-phenyl)-pyridin-2-ylethynyl]-benzo[b]thiophene-2-carboxylic acid

22.3 mL 1 N NaOH are added to a suspension of 3.0 g (7.43 mmol) methyl 5-[5-(4-chloro-phenyl)-pyridin-2-ylethynyl]-benzo[b]thiophene-2-carboxylate in 100 mL EtOH and the reaction mixture is stirred overnight at RT. The reaction solution is cooled to 0° C. and adjusted to pH 6 by dropwise addition of 1 N HCl. The precipitated product is suction filtered, washed with EtOH and dried in the air. As the product still contains educt the hydrolysis described above is repeated.

Yield: 2.7 g (93% of theoretical)

C₂₂H₁₂ClNO₂S (M=389.855)

Calc.: molpeak (M+H)⁺: 390/392 (Cl) Found: molpeak (M+H)⁺: 390/392 (Cl)

R_{f} value: 0.89 (silica gel, PE/EtOAc 1:1)

### 4d {5-[5-(4-chloro-phenyl)-pyridin-2-ylethynyl]-benzo[b]thiophen-2-yl}-methanol

2.37 g (14.62 mmol) CDI are added to a suspension of 1.9 g (4.87 mmol) 5-[5-(4-chloro-phenyl)-pyridin-2-ylethynyl]-benzo[b]thiophene-2-carboxylic acid in 40 mL DMF and the reaction mixture is heated to 50° C. overnight. After cooling to RT the reaction solution is added to a solution of 552 mg (14.62 mmol) NaBH₄ in 5 mL water in such a way that the temperature does not exceed 30° C. It is stirred for 2 h at RT, carefully combined with KHSO₄ solution until an acidic reaction is obtained, made basic with saturated Na₂CO₃ solution, extracted exhaustively with DCM, the combined organic phases are washed twice with water and dried over MgSO₄. After the desiccant and solvent have been eliminated the residue is triturated with PE, suction filtered and dried in the air.

Yield: 1.1 g (60% of theoretical)

C₂₂H₁₄ClNOS (M=375.871)

Calc.: molpeak (M+H)⁺: 376/378 (Cl) Found: molpeak (M+H)⁺: 376/378 (Cl)

HPLC-MS: 6.2 min (method B)

### 4e 2-(2-chloromethyl-benzo[b]thiophen-5-ylethynyl)-5-(4-chloro-phenyl)-pyridine

1.07 mL (15 mmol) thionyl chloride are added to a solution, cooled to 0° C., of 1.1 g (2.93 mmol) {5-[5-(4-chloro-phenyl)-pyridin-2-ylethynyl]-benzo[b]thiophen-2-yl}-methanol in 20 mL DCM and the reaction mixture is stirred for 70 h at RT. It is evaporated down i. vac., the residue is combined with semisaturated NaHCO₃ solution, extracted exhaustively with DCM, the combined organic phases are washed twice with water and dried over MgSO₄. After the desiccant and solvent have been eliminated the residue is purified by chromatography (silica gel, PE/EtOAc 7:3).

Yield: 0.65 g (56% of theoretical)

C₂₂H₁₃Cl₂NS (M=394.317)

Calc.: molpeak (M+H)⁺: 394/396/398 (2Cl) Found: molpeak (M+H)⁺: 394/396/398 (2Cl)

HPLC-MS: 7.6 min (method B)

### 4f (3R,4S)-1-{5-[5-(4-chloro-phenyl)-pyridin-2-ylethynyl]-benzo[b]thiophen-2-ylmethyl}-4-methyl-piperidine-3,4-diol

39.9 mg (0.30 mmol) (3R,4S)-4-methyl-piperidine-3,4-diol (amine A3) are added to a solution of 60 mg (0.15 mmol) 2-(2-chloromethyl-benzo[b]thiophen-5-ylethynyl)-5-(4-chloro-phenyl)-pyridine in 2 mL DMF and the reaction mixture is stirred overnight at 70° C. After filtration through an injection filter the reaction mixture is purified by HPLC

Yield: 30 mg (40% of theoretical)

C₂₈H₂₅ClN₂O₂S (M=489.029)

Calc.: molpeak (M+H)⁺: 489/491 (Cl) Found: molpeak (M+H)⁺: 489/491 (Cl)

HPLC-MS: 5.4 min (method A)

The following compounds are prepared according to the process described in Example 4f:

| Example | Structure |
|---|---|
| 4.1 | 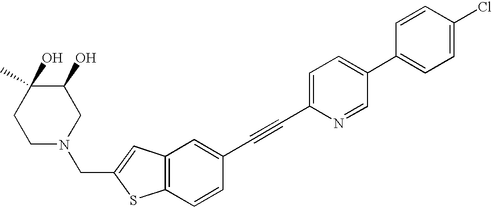 |
| 4.2 | 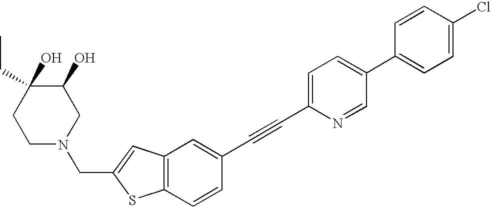 |
| 4.3 | 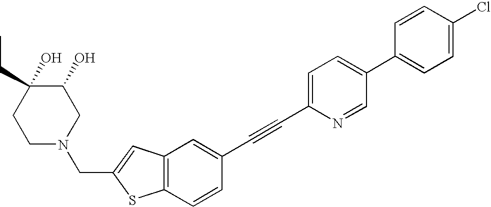 |
| 4.4 | 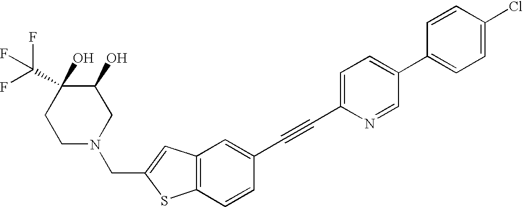 |
| 4.5 | 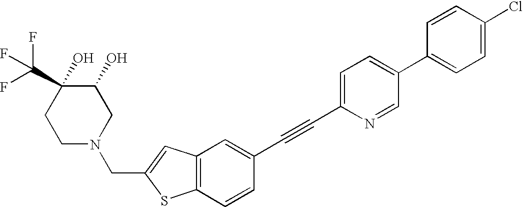 |
| 4.6 | 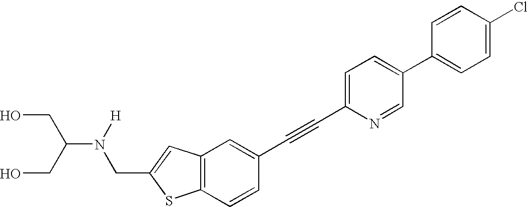 |

### EXAMPLE 5

### (3R,4S)-1-(2-{4-[5-(4-chloro-phenyl)-3-fluoro-pyridin-2-ylethynyl]-phenoxy}-ethyl)-4-methyl-piperidine-3,4-diol

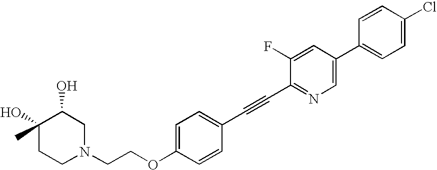

### 5a 2,5-dibromo-3-fluoro-pyridine

A solution of 1.78 g (25.80 mmol) sodium nitrite in 3.5 mL water is added dropwise at −5° C. to a solution of 6.50 g (25.80 mmol) 2,5-dibromo-pyridin-3-ylamine and 15 mL concentrated HCl (180.62 mmol) in 15 mL water and the mixture is stirred for 30 min. At 0° C. 11.41 mL (77.41 mmol) hexafluorophosphorsäure (60% in water) are added and the mixture is stirred for 1 h at 0° C. The diazonium salt formed is filtered off, washed with cold water, isopropanol and diethyl ether and dried i. vac. in the desiccator. PE (100-140° C.) is heated to 90° C., the diazonium salt is added batchwise and the mixture is stirred until no further development of gas can be detected. The reaction mixture is cooled to RT, made alkaline with saturated Na₂CO₃ solution and the aqueous phase is exhaustively extracted with MTBE. The combined organic phases are washed with saturated Na₂CO₃ solution and water, dried over MgSO₄ and evaporated down i. vac. The residue is dissolved in DCM, filtered through silica gel and the filtrate is evaporated down i. vac.

Yield: 3.30 (51% of theoretical)

C₅H₂Br₂FN (M=254.883)

Calc.: molpeak (M+H)⁺: 253/255/257 (2Br) Found: molpeak (M+H)⁺: 253/255/257 (2Br)

R_{f} value: 0.63 (silica gel, PE/EtOAc 9:1)

### 5b 5-bromo-2-[(tert-butyl-dimethyl-silanyl)-ethynyl]-3-fluoro-pyridine

Under an argon atmosphere 5.22 mL (37.67 mmol) triethylamine, 2.62 mL (13.81 mmol) tert-butyl-ethynyl-dimethyl-silane, 59.8 mg (0.31 mmol) CuI and 220.3 mg (0.31 mmol) bis-triphenylphosphane-palladium(II)-chloride is added at 15° C. to a solution of 3.20 g (12.56 mmol) 2,5-dibromo-3-fluoro-pyridine in 30 mL dry THF and the mixture is stirred for 2 h at RT. Then another 1 mL tert-butyl-ethynyl-dimethyl-silane is added and the mixture is stirred for 1 h at RT. The reaction mixture is evaporated down i. vac. and the residue taken up in EtOAc. The organic phase is washed with semisaturated NaHCO₃ solution, 5% NH₃ solution and water and dried over MgSO₄. After the desiccant and solvent have been eliminated the residue is purified by chromatography (silica gel, PE/DCM 9:1).

Yield: 1.62 g (41% of theoretical)

C₁₃H₁₇BrFNSi (M=314.269)

Calc.: molpeak (M+H)⁺: 314/316 (Br) Found: molpeak (M+H)⁺: 314/316 (Br)

HPLC-MS: 7.85 Min (method B)

### 5c 2-[(tert-butyl-dimethyl-silanyl)-ethynyl]-5-(4-chloro-phenyl)-3-fluoro-pyridine

10 mL MeOH, 10 mL 2 N Na₂CO₃ solution and 94 mg (0.13 mmol) PdCl₂(dppf) are added to a solution of 1.61 g (5.14 mmol) 5-bromo-2-[(tert-butyl-dimethyl-silanyl)-ethynyl]-3-fluoro-pyridine and 0.90 g (5.65 mmol) 4-chlorophenylboric acid in 30 mL 1,4-dioxane and the mixture is refluxed for 15 min. The reaction mixture is evaporated down i. vac. and diluted with EtOAc. The organic phase is washed with water and semisaturated NaHCO₃ solution and dried over Na₂SO₄. After the desiccant and solvent have been eliminated the residue is purified by chromatography (silica gel, PE/DCM 1:1).

Yield: 1.25 g (70% of theoretical)

C₁₉H₂₁ClFNSi (M=345.913)

Calc.: molpeak (M+H)⁺: 346/348 (Cl) Found: molpeak (M+H)⁺: 346/348 (Cl)

HPLC-MS: 8.83 Min (method B)

### 5d 5-(4-chloro-phenyl)-2-ethynyl-3-fluoro-pyridine

1.14 g (3.61 mmol) TBAF are added at RT to a solution of 1.25 g (3.61 mmol) 2-[(tert-butyl-dimethyl-silanyl)-ethynyl]-5-(4-chloro-phenyl)-3-fluoro-pyridine in 30 mL DCM and the mixture is stirred for 2 h. The organic phase is washed with water and dried over Na₂SO₄. After the desiccant and solvent have been eliminated the residue is stirred with PE, the precipitate is filtered off, washed with PE and dried in the air.

Yield: 0.72 g (86% of theoretical)

C₁₃H₇ClFN (M=231.653)

Calc.: molpeak (M+H)⁺: 232/234 (Cl) Found: molpeak (M+H)⁺: 232/234 (Cl)

HPLC-MS: 5.81 Min (method B)

### 5e 2-{4-[5-(4-chloro-phenyl)-3-fluoro-pyridin-2-ylethynyl]-phenoxy}-ethanol

Under an argon atmosphere 14.8 mg (0.08 mmol) CuI is added to a solution of 0.82 g (3.11 mmol) 2-(4-iodo-2-methyl-phenoxy)-ethanol, 0.72 g (2.11 mmol) 5-(4-chloro-phenyl)-2-ethynyl-3-fluoro-pyridine, 57 mg (0.078 mmol) PdCl₂(dppf) and 0.86 mL (6.22 mmol) triethylamine in 20 mL THF and the mixture is stirred for 16 h at RT. The reaction mixture is combined with EtOAc, the precipitate is filtered off and washed with EtOAc. The filtrate and the organic wash solutions are washed with 5% NH₃ solution and water and dried over MgSO₄. After the desiccant and solvent have been eliminated the residue is purified by chromatography (silica gel, PE/EtOAc 1:1). (silica gel, petroleum ether/EtOAc 1:1)

Yield: 0.20 g (18% of theoretical)

C₂₁H₁₅ClFNO₂ (M=367.800)

Calc.: molpeak (M+H)⁺: 368/370 (Cl) Found: molpeak (M+H)⁺: 368/370 (Cl)

HPLC-MS: 5.9 min (method B)

### 5f 2-{4-[5-(4-chloro-phenyl)-3-fluoro-pyridin-2-ylethynyl]-phenoxy}-ethyl methanesulphonate

63 μL (0.82 mmol) methanesulphonic acid chloride is added dropwise at 0° C. to a solution of 0.20 g (0.54 mmol) 2-{4-[5-(4-chloro-phenyl)-3-fluoro-pyridin-2-ylethynyl]-phenoxy}-ethanol and 0.11 mL (0.82 mmol) triethylamine in 10 mL DCM and the mixture is stirred for 16 h at RT. Another 0.11 mL (0.82 mmol) triethylamine and 63 μL (0.82 mmol) methanesulphonic acid chloride are added and the mixture is stirred for 8 h. The reaction mixture is diluted with DCM, the organic phase is washed with water and dilute NaHCO₃ solution, dried over MgSO₄ and evaporated down i. vac.

Yield: 0.24 g (99% of theoretical)

C₂₂H₁₇ClFNO₄S (M=445.892)

Calc.: molpeak (M+H)⁺: 446/448 (Cl) Found: molpeak (M+H)⁺: 446/448 (Cl)

HPLC-MS: 6.2 min (method B)

### 5g (3R,4S)-1-(2-{4-[5-(4-chloro-phenyl)-3-fluoro-pyridin-2-ylethynyl]-phenoxy}-ethyl)-4-methyl-piperidine-3,4-diol

The product is obtained analogously to Example 4f starting from 60 mg (0.135 mmol) methanesulphonate 2-{4-[5-(4-chloro-phenyl)-3-fluoro-pyridin-2-ylethynyl]-phenoxy}-ethyl and 35.3 mg (0.269 mmol) (3R,4S)-4-methyl-piperidine-3,4-diol (amine A3).

Yield: 47 mg (73% of theoretical)

C₂₇H₂₆ClFN₂O₃ (M=480.958)

Calc.: molpeak (M+H)⁺: 481/483 (Cl) Found: molpeak (M+H)⁺: 481/483 (Cl)

HPLC-MS: 5.2 min (method A)

The following compounds are prepared according to the process described in Example 5g:

| Example | Structure |
|---|---|
| 5.1 | 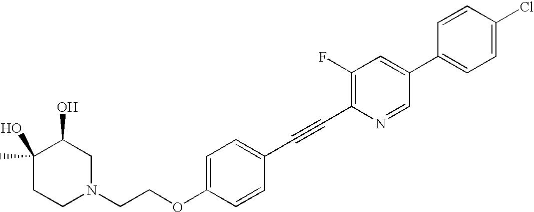 |
| 5.2 | 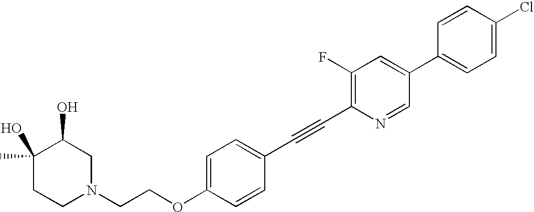 |
| 5.3 | 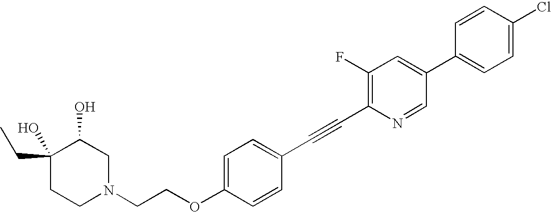 |
| 5.4 | 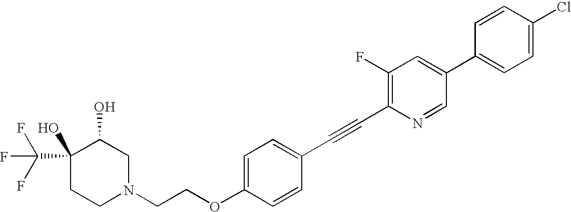 |
| 5.5 | 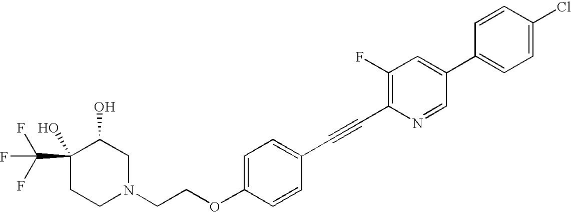 |
| 5.6 | 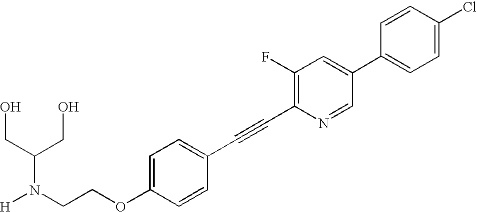 |

### EXAMPLE 5.7

### 1-[(S)-1-(2-{4-[5-(4-chloro-phenyl)-3-fluoro-pyridin-2-ylethynyl]-phenoxy}-ethyl)-pyrrolidin-2-yl]-cyclopropanol

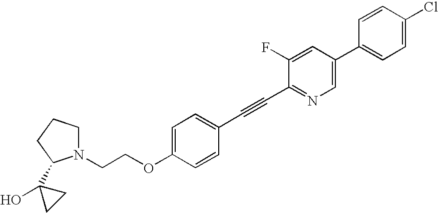

The product is obtained analogously to Example 4f starting from 60 mg (0.135 mmol) 2-{4-[5-(4-chloro-phenyl)-3-fluoro-pyridin-2-ylethynyl]-phenoxy}-ethyl methanesulphonate and 34.2 mg (0.269 mmol) (S)-1-pyrrolidin-2-yl-cyclopropanol (amine A12).

Yield: 12 mg (19% of theoretical)

C₂₈H₂₆ClFN₂O₂ (M=476.969)

Calc.: molpeak (M+H)⁺: 477/479 (Cl) Found: molpeak (M+H)⁺: 477/479 (Cl)

HPLC-MS: 5.6 min (method A)

### EXAMPLE 6

### 1-[(S)-1-(2-{2-bromo-4-[5-(4-chloro-phenyl)-3-fluoro-pyridin-2-ylethynyl]-phenoxy}-ethyl)-pyrrolidin-2-yl]-cyclopropanol

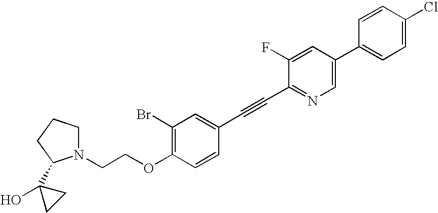

### 6a 2-(2-bromo-4-iodo-phenoxy)-ethanol

The product is prepared analogously to Example 1a from 30.8 g (103 mmol) 2-bromo-4-iodo-phenol.

Yield: 18.5 g (52% of theoretical)

C₈H₈BrIO₂ (M=342.956)

R_{f} value: 0.18 (silica gel, PE/EtOAc 4:1)

### 6b 2-{2-bromo-4-[5-(4-chloro-phenyl)-3-fluoro-pyridin-2-ylethynyl]-phenoxy}-ethanol

The product is prepared analogously to Example 5e starting from 1.5 g (4.37 mmol) 2-(2-bromo-4-iodo-phenoxy)-ethanol and 1.01 g (4.37 mmol) 5-(4-chloro-phenyl)-2-ethynyl-3-fluoro-pyridine.

Yield: 1.60 g (82% of theoretical)

C₂₁H₁₄BrClFNO₂ (M=446.697)

Calc.: molpeak (M+H)⁺: 446/448/450 (BrCl) Found: molpeak (M+H)⁺: 446/448/450 (BrCl)

HPLC-MS: 6.4 min (method B)

### 6c 2-{2-bromo-4-[5-(4-chloro-phenyl)-3-fluoro-pyridin-2-ylethynyl]-phenoxy}-ethyl methanesulphonate

The product is prepared analogously to Example 5f starting from 1.6 g (3.58 mmol) 2-{2-bromo-4-[5-(4-chloro-phenyl)-3-fluoro-pyridin-2-ylethynyl]-phenoxy}-ethanol.

Yield: 1.00 g (53% of theoretical)

C₂₂H₁₆BrClFNO₄S (M=524.788)

Calc.: molpeak (M+H)⁺: 524/526/528 (BrCl) Found: molpeak (M+H)⁺: 524/526/528 (BrCl)

HPLC-MS: 6.6 min (method B)

### 6d 1-[(S)-1-(2-{2-bromo-4-[5-(4-chloro-phenyl)-3-fluoro-pyridin-2-ylethynyl]-phenoxy}-ethyl)-pyrrolidin-2-yl]-cyclopropanol

The product is obtained analogously to Example 4f starting from 60.0 mg (0.114 mmol) 2-{2-bromo-4-[5-(4-chloro-phenyl)-3-fluoro-pyridin-2-ylethynyl]-phenoxy}-ethyl methanesulphonate and 29.1 mg (0.229 mmol) (S)-1-pyrrolidin-2-yl-cyclopropanol (amine A12).

Yield: 11 mg (17% of theoretical)

C₂₈H₂₅BrClFN₂O₂ (M=555.865)

Calc.: molpeak (M+H)⁺: 555/557/559 (BrCl) Found: molpeak (M+H)⁺: 555/557/559 (BrCl)

HPLC-MS: 5.7 min (method A)

The following compounds are prepared according to the process described in Example 6d:

| Example | Structure |
|---|---|
| 6.1 | 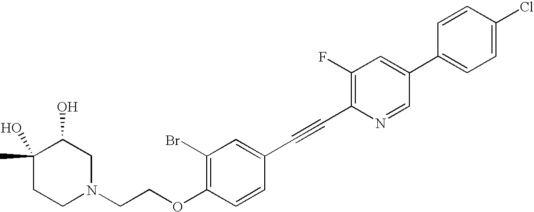 |
| 6.2 | 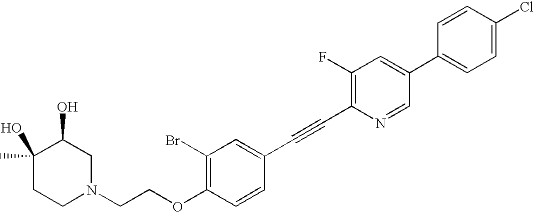 |
| 6.3 | 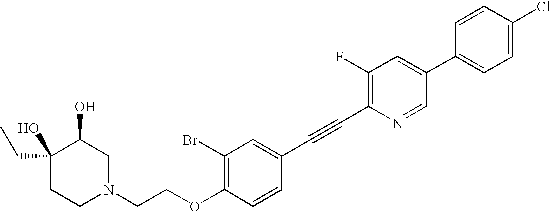 |
| 6.4 | 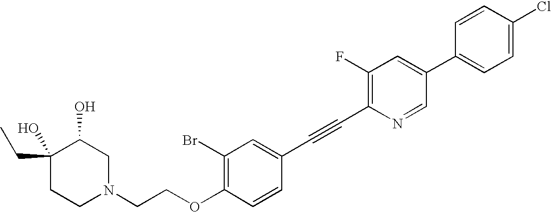 |
| 6.5 | 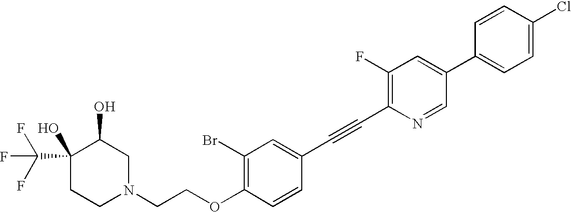 |
| 6.6 | 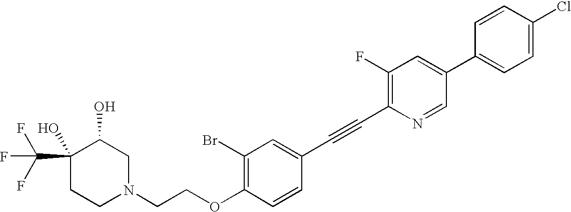 |
| 6.7 | 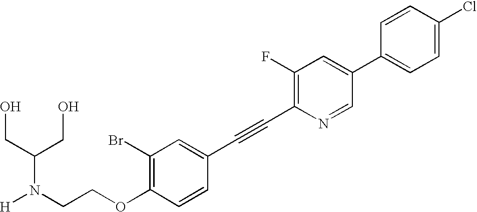 |

### EXAMPLE 7

### 1-[(S)-1-(2-{4-[5-(4-chloro-phenyl)-3-fluoro-pyridin-2-ylethynyl]-2-methyl-phenoxy}-ethyl)-pyrrolidin-2-yl]-cyclopropanol

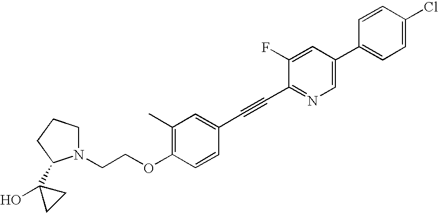

### 7a 2-(4-iodo-2-methyl-phenoxy)-ethanol

The product is prepared analogously to Example 1a starting from 10 g (42.7 mmol) 4-iodo-2-methyl-phenol.

Yield: 11.4 g (96% of theoretical)

C₉H₁₁IO₂ (M=278.087)

R_{f} value: 0.40 (silica gel, PE/EtOAc 3:2)

### 7b 2-{4-[5-(4-chloro-phenyl)-3-fluoro-pyridin-2-ylethynyl]-2-methyl-phenoxy}-ethanol

The product is prepared analogously to Example 5e starting from 1.5 g (5.39 mmol) 2-(4-iodo-2-methyl-phenoxy)-ethanol and 1.25 g (5.39 mmol) 5-(4-chloro-phenyl)-2-ethynyl-3-fluoro-pyridine.

Yield: 1.40 g (68% of theoretical)

C₂₂H₁₇ClFNO₂ (M=381.827)

Calc.: molpeak (M+H)⁺: 382/384 (Cl) Found: molpeak (M+H)⁺: 382/384 (Cl)

HPLC-MS: 6.3 min (method B)

### 7c 2-{4-[5-(4-chloro-phenyl)-3-fluoro-pyridin-2-ylethynyl]-2-methyl-phenoxy}-ethyl methanesulphonate

The product is prepared analogously to Example 5f starting from 1.4 g (3.67 mmol) 2-{4-[5-(4-chloro-phenyl)-3-fluoro-pyridin-2-ylethynyl]-2-methyl-phenoxy}-ethanol.

Yield: 0.77 g (46% of theoretical)

C₂₃H₁₉ClFNO₄S (M=459.918)

Calc.: molpeak (M+H)⁺: 460/462 (Cl) Found: molpeak (M+H)⁺: 460/462 (Cl)

HPLC-MS: 6.5 min (method B)

### 7d 1-[(S)-1-(2-{4-[5-(4-chloro-phenyl)-3-fluoro-pyridin-2-ylethynyl]-2-methyl-phenoxy}-ethyl)-pyrrolidin-2-yl]-cyclopropanol

The product is obtained analogously to Example 4f starting from 60.0 mg (0.130 mmol) 2-{4-[5-(4-chloro-phenyl)-3-fluoro-pyridin-2-ylethynyl]-2-methyl-phenoxy}-ethyl methanesulphonate and 33.2 mg (0.261 mmol) (S)-1-pyrrolidin-2-yl-cyclopropanol (amine A12).

Yield: 22 mg (34% of theoretical)

C₂₉H₂₈ClFN₂O₂ (M=490.996)

Calc.: molpeak (M+H)⁺: 491/493 (Cl) Found: molpeak (M+H)⁺: 491/493 (Cl)

HPLC-MS: 5.8 min (method A)

The following compounds are prepared according to the process described in Example 7d:

| Example | Structure |
|---|---|
| 7.1 | 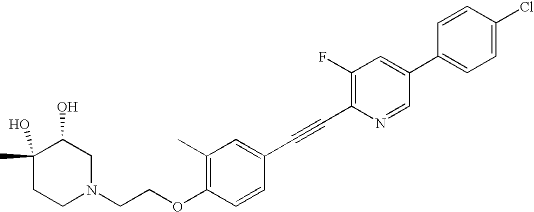 |
| 7.2 | 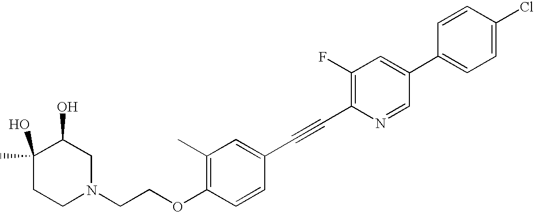 |
| 7.3 | 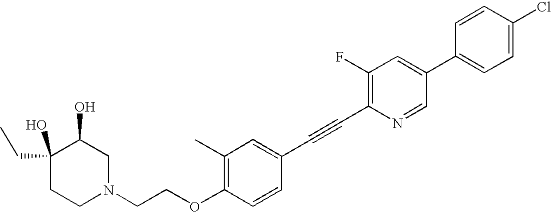 |
| 7.4 | 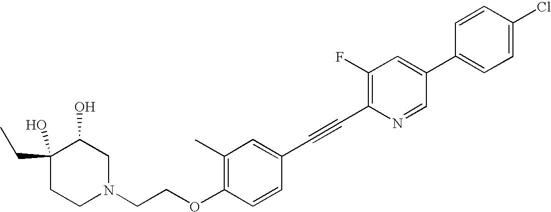 |
| 7.5 | 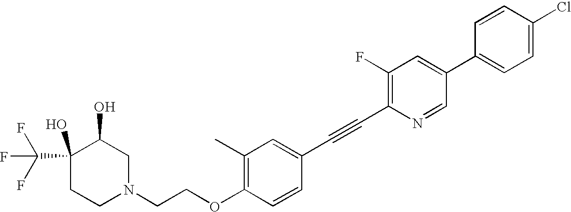 |
| 7.6 | 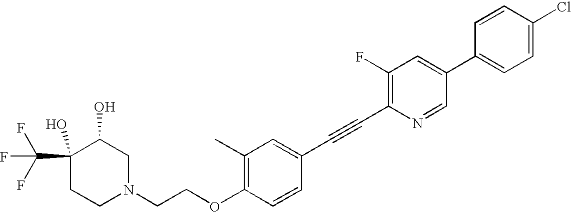 |
| 7.7 | 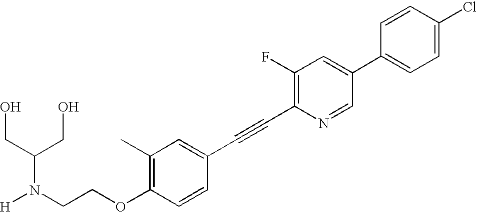 |

### EXAMPLE 8

### (3R,4S)-1-((E)-3-{5-[5-(4-chloro-phenyl)-pyridin-2-ylethynyl]-pyridin-2-yl}-allyl)-4-methyl-piperidine-3,4-diol

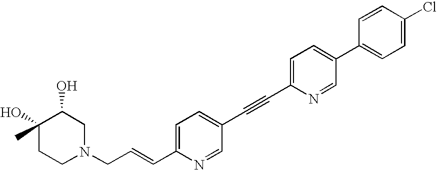

### 8a (E)-3-(5-bromo-pyridin-2-yl)-prop-2-en-1-ol

A solution of 1.0 g (4.71 mmol) 3-(5-bromo-pyridin-2-yl)-prop-2-yn-1-ol in 5 mL THF is added dropwise to a suspension of 4.7 mL (4.7 mmol, 1 M in THF) lithium aluminium hydride solution in 20 mL THF cooled to −5° C. so that the temperature does not exceed 0° C. After the addition has ended the mixture is stirred for a further 2 h at 0° C. Excess lithium aluminium hydride is decomposed by the careful addition of 0.13 mL water, 0.13 mL 15% NaOH and 0.38 mL water. The precipitate is filtered and the organic phase is dried over MgSO₄. After the desiccant and solvent have been eliminated the residue is reacted further without purification.

Yield: 0.91 g (63% of theoretical)

C₈H₈BrNO (M=214.059)

Calc.: molpeak (M+H)⁺: 214/216 (Br) Found: molpeak (M+H)⁺: 214/216 (Br)

HPLC-MS: 4.2 min (method B)

### 8b (E)-3-(5-iodo-pyridin-2-yl)-prop-2-en-1-ol

Under an argon atmosphere 81 mg (0.43 mmol) CuI, 1.27 g (8.5 mmol) NaI and 90 μL N,N′-dimethylethylenediamine are added to a solution of 910 mg (4.25 mmol) (E)-3-(5-bromo-pyridin-2-yl)-prop-2-en-1-ol in 4.5 mL 1,4-dioxane and the reaction mixture is refluxed overnight. After cooling it is combined with water, exhaustively extracted with EtOAc and the combined organic phases are dried over MgSO₄. After the desiccant and solvent have been eliminated the residue is reacted further without purification.

Yield: 870 mg (78% of theoretical)

C₈H₈INO (M=261.060)

Calc.: molpeak (M+H)⁺: 262 Found: molpeak (M+H)⁺: 262

HPLC-MS: 4.2 min (method B)

### 8c (E)-3-{5-[5-(4-chloro-phenyl)-pyridin-2-ylethynyl]-pyridin-2-yl}-prop-2-en-1-ol

A solution of 870 mg (3.33 mmol) (E)-3-(5-iodo-pyridin-2-yl)-prop-2-en-1-ol, 712 mg (3.33 mmol) 5-(4-chloro-phenyl)-2-ethynyl-pyridine and 0.97 mL (7.0 mmol) triethylamine in 10 mL dry acetonitrile is evacuated three times and then gassed with argon. Then 70 mg (0.37 mmol) CuI and 300 mg (0.37 mmol) PdCl₂(dppf) are added. The reaction mixture is stirred overnight at RT. The precipitated product is filtered, washed with a little acetonitrile and dried in the circulating air dryer at 50° C. until the weight remains constant.

Yield: 980 mg (85% of theoretical)

C₂₁H₁₅ClN₂O (M=346.809)

Calc.: molpeak (M+H)⁺: 347/349 (Cl) Found: molpeak (M+H)⁺: 347/349 (Cl)

HPLC-MS: 5.6 min (method B)

### 8d (E)-3-{5-[5-(4-chloro-phenyl)-pyridin-2-ylethynyl]-pyridin-2-yl}-allyl)-chloride

A solution of 160 μL thionyl chloride in 5 mL DCM is added dropwise to a solution of 450 mg (1.30 mmol) (E)-3-{5-[5-(4-chloro-phenyl)-pyridin-2-ylethynyl]-pyridin-2-yl}-prop-2-en-1-ol in 25 mL DCM cooled to −10° C. The reaction solution is stirred for a further 30 min at 0° C. and stirred overnight at RT. The reaction mixture is combined with 50 mL semisaturated NaHCO₃ solution, exhaustively extracted with DCM, the combined organic phases are washed twice with water and dried over MgSO₄. After the desiccant and solvent have been eliminated the residue is reacted further without purification.

Yield: 450 mg (95% of theoretical)

C₂₁H₁₄Cl₂N₂ (M=365.255)

Calc.: molpeak (M+H)⁺: 365/367/369 (2 Cl) Found: molpeak (M+H)⁺: 365/367/369 (2 Cl)

HPLC-MS: 6.8 min (method B)

The following compounds are prepared according to the process described in Example 3g:

| Example | Structure |
|---|---|
| 8 | 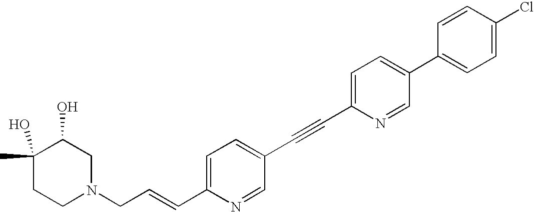 |
| 8.1 | 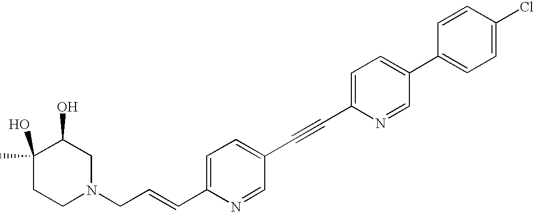 |
| 8.2 | 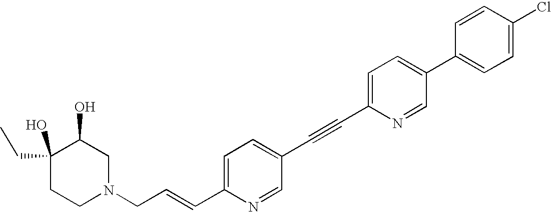 |
| 8.3 | 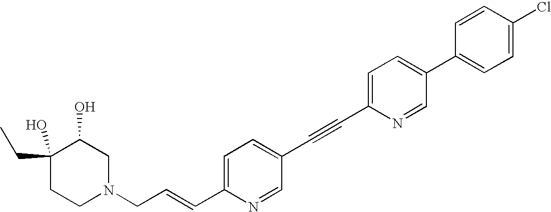 |
| 8.4 | 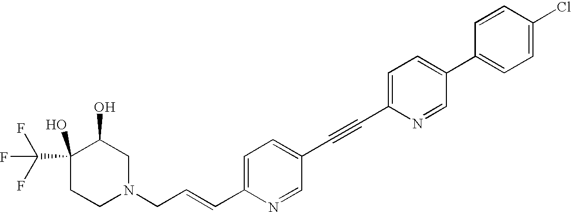 |
| 8.5 | 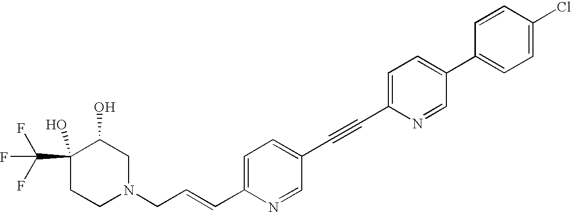 |
| 8.6 | 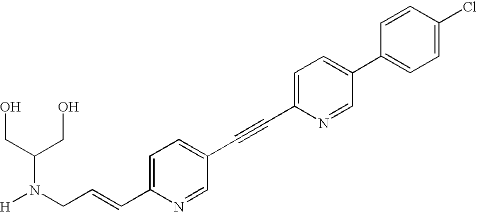 |

Some test methods for determining an MCH-receptor antagonistic activity will now be described. In addition, other test methods known to the skilled man may be used, e.g. by inhibiting the MCH-receptor-mediated inhibition of cAMP production, as described by Hoogduijn M et al. in “Melanin-concentrating hormone and its receptor are expressed and functional in human skin”, Biochem. Biophys. Res Commun. 296 (2002) 698-701 and by biosensory measurement of the binding of MCH to the MCH receptor in the presence of antagonistic substances by plasmon resonance, as described by Karlsson OP and Lofas S. in “Flow-Mediated On-Surface Reconstitution of G-Protein Coupled Receptors for Applications in Surface Plasmon Resonance Biosensors”, Anal. Biochem. 300 (2002), 132-138. Other methods of testing antagonistic activity to MCH receptors are contained in the references and patent documents mentioned hereinbefore, and the description of the test methods used is hereby incorporated in this application.

MCH-1 Receptor Binding Test

Method: MCH binding to hMCH-1R transfected cells

Species: Human

Test cell: hMCH-1R stably transfected into CHO/Galpha16 cells

Results: IC50 values

Membranes from CHO/Galpha16 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/ml aprotinin, 1 μg/ml leupeptin and 1 μM phosphoramidone) to a concentration of 5 to 15 μg/ml.

200 microlitres of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 microlitres. After the incubation the reaction is filtered using a cell harvester through 0.5% PEI treated fibreglass filters (GF/B, Unifilter Packard). The membrane-bound radioactivity retained on the filter is then determined after the addition of scintillator substance (Packard Microscint 20) in a measuring device (TopCount of Packard).

The non-specific binding is defined as bound radioactivity in the presence of 1 micromolar MCH during the incubation period.

The analysis of the concentration binding curve is carried out on the assumption of one receptor binding site.

Standard:

Non-labelled MCH competes with labelled ¹²⁵I-MCH for the receptor binding with an IC50 value of between 0.06 and 0.15 nM.

The KD value of the radioligand is 0.156 nM.

MCH-1 Receptor-Coupled Ca²⁺ Mobilisation Test

| Method: | Calcium mobilisation test with human MCH (FLIPR³⁸⁴) | |
|---|---|---|
| Species: | Human | |
| Test cells: | CHO/Galpha 16 cells stably transfected with hMCH-R1 | |
| Results: | 1st measurement:: % stimulation of the | |
| | reference (MCH 10⁻⁶ M) | |
| | 2nd measurement: pKB value | |
| Reagents: | HBSS (10×) | (GIBCO) |
| | HEPES buffer (1M) | (GIBCO) |
| | Pluronic F-127 | (Molecular Probes) |
| | Fluo-4 | (Molecular Probes) |
| | Probenecid | (Sigma) |
| | MCH | (Bachem) |
| | bovine serum albumin | (Serva) |
| | (protease-free) | |
| | DMSO | (Serva) |
| | Ham's F12 | (BioWhittaker) |
| | PCS | (BioWhittaker) |
| | L-Glutamine | (GIBCO) |
| | Hygromycin B | (GIBCO) |
| | PENStrep | (BioWhittaker) |
| | Zeocin | (Invitrogen) |

Clonal CHO/Galpha16 hMCH-R1 cells are cultivated in Ham's F12 cell culture medium (with L-glutamine; BioWhittaker; Cat.No.: BE12-615F). This contains per 500 ml 10% FCS, 1% PENStrep, 5 ml L-glutamine (200 mM stock solution), 3 ml hygromycin B (50 mg/ml in PBS) and 1.25 ml zeocin (100 μg/ml stock solution). One day before the experiment the cells are plated on a 384-well microtitre plate (black-walled with a transparent base, made by Costar) in a density of 2500 cells per cavity and cultivated in the above medium overnight at 37° C., 5% CO₂ and 95% relative humidity. On the day of the experiment the cells are incubated with cell culture medium to which 2 mM Fluo-4 and 4.6 mM Probenicid have been added, at 37° C. for 45 minutes. After charging with fluorescent dye the cells are washed four times with Hanks buffer solution (1×HBSS, 20 mM HEPES), which has been combined with 0.07% Probenicid. The test substances are diluted in Hanks buffer solution, combined with 2.5% DMSO. The background fluorescence of non-stimulated cells is measured in the presence of substance in the 384-well microtitre plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR³⁸⁴ apparatus.

Data Analysis:

1st measurement: The cellular Ca²⁺ mobilisation is measured as the peak of the relative fluorescence minus the background and is expressed as the percentage of the maximum signal of the reference (MCH 10⁻⁶M). This measurement serves to identify any possible agonistic effect of a test substance.

2nd measurement: The cellular Ca²⁺ mobilisation is measured as the peak of the relative fluorescence minus the background and is expressed as the percentage of the maximum signal of the reference (MCH 10⁻⁶M, signal is standardised to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:
pKB=log(EC_{50(testsubstance+MCH})/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

The following IC50 values were determined using the MCH-1 receptor binding test described above:

| Compound | | |
|---|---|---|
| according to | | IC50 |
| Example No. | Name of substance | value |
| 2.20 | 1-{[(2-{4-[5-(4-chloro-phenyl)-pyridin-2- | 13.8 nM |
| | ylethynyl]-phenoxy}-ethyl)-(3-hydroxy- | |
| | propyl)-amino]-methyl}-cyclopropanol | |
| 1.5 | (3R,4S)-1-(2-{4-[5-(4-chloro-phenyl)-pyridin- | 10.9 nM |
| | 2-ylethynyl]-2-methyl-phenoxy}-ethyl)-3- | |
| | methyl-piperidine-3,4-diol | |

Some examples of formulations will be described hereinafter, wherein the term “active substance” denotes one or more compounds according to the invention, including their salts. In the case of one of the combinations with one or more active substances described, the term “active substance” also includes the additional active substances.

### EXAMPLE A

Capsules for Powder Inhalation Containing 1 mg Active Substance

Composition:

1 capsule for powder inhalation contains:

| | | |
|---|---|---|
| active substance | 1.0 | mg |
| lactose | 20.0 | mg |
| hard gelatine capsules | 50.0 | mg |
| | 71.0 | mg |

Method of Preparation:

The active substance is ground to the particle size required for inhalation. The ground active substance is homogeneously mixed with the lactose. The mixture is packed into hard gelatine capsules.

### EXAMPLE B

Inhalable Solution for Respimat® Containing 1 mg Active Substance

Composition:

1 spray contains:

| | | |
|---|---|---|
| active substance | 1.0 | mg |
| benzalkonium chloride | 0.002 | mg |
| disodium edetate | 0.0075 | mg |
| purified water ad | 15.0 | μl |

Method of Preparation:

The active substance and benzalkonium chloride are dissolved in water and packed into Respimat® cartridges.

### EXAMPLE C

Inhalable Solution for Nebulisers Containing 1 mg Active Substance

Composition:

1 vial contains:

| | | |
|---|---|---|
| active substance | 0.1 | g |
| sodium chloride | 0.18 | g |
| benzalkonium chloride | 0.002 | g |
| purified water ad | 20.0 | ml |

Method of Preparation:

The active substance, sodium chloride and benzalkonium chloride are dissolved in water.

### EXAMPLE D

Propellant Type Metered Dose Aerosol Containing 1 mg Active Substance

Composition:

1 spray contains:

| active substance | 1.0 | mg |
|---|---|---|
| lecithin | 0.1% | |
| propellant gas ad | 50.0 | μl |

Method of Preparation:

The micronised active substance is homogeneously suspended in the mixture of lecithin and propellant gas. The suspension is transferred into a pressurised container with a metering valve.

### EXAMPLE E

Nasal Spray Containing 1 mg Active Substance

Composition:

| | | |
|---|---|---|
| active substance | 1.0 | mg |
| sodium chloride | 0.9 | mg |
| benzalkonium chloride | 0.025 | mg |
| disodium edetate | 0.05 | mg |
| purified water ad | 0.1 | ml |

Method of Preparation:

The active substance and the excipients are dissolved in water and transferred into a corresponding container.

### EXAMPLE F

Injectable Solution Containing 5 mg of Active Substance Per 5 ml

Composition:

| | | |
|---|---|---|
| active substance | 5 | mg |
| glucose | 250 | mg |
| human serum albumin | 10 | mg |
| glycofurol | 250 | mg |
| water for injections ad | 5 | ml |

Preparation:

Glycofurol and glucose are dissolved in water for injections (WfI); human serum albumin is added; active ingredient is dissolved with heating; made up to specified volume with WfI; transferred into ampoules under nitrogen gas.

### EXAMPLE G

Injectable Solution Containing 100 mg of Active Substance Per 20 ml

Composition:

| | | |
|---|---|---|
| active substance | 100 | mg |
| monopotassium dihydrogen | 12 | mg |
| phosphate = KH₂PO₄ | | |
| disodium hydrogen | 2 | mg |
| phosphate = Na₂HPO₄.₂O | | |
| sodium chloride | 180 | mg |
| human serum albumin | 50 | mg |
| Polysorbate 80 | 20 | mg |
| water for injections ad | 20 | ml |

Preparation:

Polysorbate 80, sodium chloride, monopotassium dihydrogen phosphate and disodium hydrogen phosphate are dissolved in water for injections (WfI); human serum albumin is added; active ingredient is dissolved with heating; made up to specified volume with WfI; transferred into ampoules.

### EXAMPLE H

Lyophilisate Containing 10 mg of Active Substance

Composition:

| | | |
|---|---|---|
| Active substance | 10 | mg |
| Mannitol | 300 | mg |
| human serum albumin | 20 | mg |

Preparation:

Mannitol is dissolved in water for injections (WfI); human serum albumin is added; active ingredient is dissolved with heating; made up to specified volume with WfI; transferred into vials; freeze-dried.

Solvent for Lyophilisate:

| | | |
|---|---|---|
| Polysorbate 80 = Tween 80 | 20 | mg |
| mannitol | 200 | mg |
| water for injections ad | 10 | ml |

Preparation:

Polysorbate 80 and mannitol are dissolved in water for injections (WfI); transferred into ampoules.

### EXAMPLE I

Tablets Containing 20 mg of Active Substance

Composition:

| | | |
|---|---|---|
| active substance | 20 | mg |
| lactose | 120 | mg |
| maize starch | 40 | mg |
| magnesium stearate | 2 | mg |
| Povidone K 25 | 18 | mg |

Preparation:

Active substance, lactose and maize starch are homogeneously mixed; granulated with an aqueous solution of Povidone; mixed with magnesium stearate; compressed in a tablet press; weight of tablet 200 mg.

### EXAMPLE J

Capsules Containing 20 mg Active Substance

Composition:

| | | |
|---|---|---|
| active substance | 20 | m9 |
| maize starch | 80 | mg |
| highly dispersed silica | 5 | mg |
| magnesium stearate | 2.5 | mg |

Preparation:

Active substance, maize starch and silica are homogeneously mixed; mixed with magnesium stearate; the mixture is packed into size 3 hard gelatine capsules in a capsule filling machine.

### EXAMPLE K

Suppositories Containing 50 mg of Active Substance

Composition:

| | | |
|---|---|---|
| active substance | 50 | mg |
| hard fat (Adeps solidus) q.s. ad | 1700 | mg |

Preparation:

Hard fat is melted at about 38° C.; ground active substance is homogeneously dispersed in the molten hard fat; after cooling to about 35° C. it is poured into chilled moulds.

### EXAMPLE L

Injectable Solution Containing 10 mg of Active Substance Per 1 ml

Composition:

| | | |
|---|---|---|
| active substance | 10 | mg |
| mannitol | 50 | mg |
| human serum albumin | 10 | mg |
| water for injections ad | 1 | ml |

Preparation:

Mannitol is dissolved in water for injections (WfI); human serum albumin is added; active ingredient is dissolved with heating; made up to specified volume with WfI; transferred into ampoules under nitrogen gas.

1. A compound of formula I 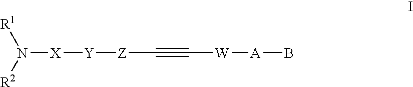
wherein:
R¹ is C₃₋₆-alkenyl, C₃₋₆-alkynyl, (hydroxy-C₃₋₇-cycloalkyl)-C₁₋₃-alkyl, oxa-C₄₋₇-cycloalkyl, or dihydroxy-C₃₋₇-alkyl, each optionally independently mono- or polysubstituted by substituents selected from halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino, or di-(C₁₋₄-alkyl)-amino, wherein the alkyl, alkoxy, and cycloalkyl groups thereof optionally comprise one or more identical or different halogen or hydroxy substituents,
R² is independently R¹ or is H, C₁₋₈-alkyl, C₃₋₇-cycloalkyl, or a phenyl or pyridinyl group each optionally mono- or polysubstituted by identical or different groups R²⁰ and/or monosubstituted by nitro, wherein the alkyl or cycloalkyl group thereof are optionally mono- or polysubstituted by identical or different groups R¹¹, and a —CH₂— group in position 3 or 4 of a 5-, 6- or 7-membered cycloalkyl group is optionally replaced by —O—, —S—, or —NR¹³—, or
R¹ and R² together with the N atom to which they are bound form a heterocyclic group selected from dihydroxy-cyclo-C₄₋₇-alkylene-imino, (hydroxy-C₁₋₄-alkyl)-hydroxy-cyclo-C₃₋₇-alkylene-imino, or (hydroxy-C₁₋₃-alkyl)cyclo-C₃₋₇-alkylene-imino wherein the C₁₋₃-alkyl group thereof is substituted by one or more identical or different C₁₋₃-alkyl groups optionally joined together to form a C₃₋₇-cycloalkyl group, wherein each heterocyclic group is optionally independently mono- or polysubstituted by substituents selected from halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino, and di-C₁₋₄-alkyl)-amino, wherein the alkyl, alkoxy, and cycloalkyl groups thereof optionally comprise one or more identical or different halogen or hydroxy substituents;
X is a C₁₋₄-alkylene bridge, wherein if X is a C₂₋₄-alkylene bridge, one or two C atoms thereof are optionally monosubstituted by R¹⁰, or if X is a C₃₋₄-alkylene bridge, a —CH₂—CH₂— group not directly adjacent to the N atom of the R¹R²N— group is replaced by —CH═CH—, —C≡C—, —CH₂—O—, —CH₂—S—, or —CH₂—NR⁴—, wherein X optionally comprises a substituent selected from C₂₋₆-alkenyl, C₂₋₆-alkynyl, C₃₋₇-cycloalkyl, and C₃₋₇-cycloalkyl-C₁₋₃-alkyl, or one, two, or three identical or different C₁₋₄-alkyl substituents, wherein two alkyl groups thereof are optionally joined together to form a 3- to 7-membered cyclic group or an alkyl and an alkenyl group are optionally joined together to form a 5- to 7-membered cyclic group;
W and Z are each independently a single bond or a C₁₋₂-alkylene bridge, wherein two adjacent C atoms are optionally joined together with an additional C₁₋₄-alkylene bridge, and one or two C atoms are optionally independently substituted by one or two identical or different C₁₋₃-alkyl groups, wherein two alkyl groups are optionally joined together to form a carbocyclic ring;
Y and A are each independently phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, thienyl, furanyl, benzothienyl, or benzofuranyl, each optionally mono- or polysubstituted at one or more C atoms by identical or different groups R²⁰, and, in the case of a phenyl ring, optionally additionally monosubstituted by nitro, and/or one or more NH groups optionally substituted by R²¹;
B is independently Y, A, or C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, or C₃₋₇-cycloalkyl-C₁₋₃-alkynyl, wherein one or more C atoms are optionally independently mono- or polysubstituted by halogen and/or monosubstituted by hydroxy or cyano and/or cyclic groups thereof are optionally mono- or polysubstituted by identical or different groups R²⁰;
Cy is a saturated 3- to 7-membered carbocyclic group, an unsaturated 4- to 7-membered carbocyclic group, a phenyl group, a saturated 4- to 7-membered or unsaturated 5- to 7-membered heterocyclic group with an N, O, or S atom as heteroatom, a saturated or unsaturated 5- to 7-membered heterocyclic group with two or more N atoms or with one or two N atoms and an O or S atom as heteroatoms, or an aromatic heterocyclic 5- or 6-membered group with one or more identical or different heteroatoms selected from N, O, and/or S, wherein the saturated 6- or 7-membered groups thereof are optionally bridged ring systems with an imino, (C₁₋₄-alkyl)-imino, methylene, (C₁₋₄-alkyl)-methylene, or di-(C₁₋₄-alkyl)-methylene bridge, and the cyclic groups thereof are optionally mono- or polysubstituted at one or more C atoms by identical or different groups R²⁰, and, in the case of a phenyl group, are optionally additionally monosubstituted by nitro, and/or one or more NH groups are substituted by R²¹;
R⁴ is H, C₁₋₄-alkyl, C₃₋₇-cycloalkyl, or C₃₋₇-cycloalkyl-C₁₋₃-alkyl;
R¹⁰ is hydroxy, ω-hydroxy-C₁₋₃-alkyl, C₁₋₄-alkoxy, or C₁₋₄-alkoxy-C₁₋₃-alkyl;
R¹¹ is halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N, R¹⁸R¹⁹N—CO—, or Cy, wherein one or more C atoms thereof are optionally independently mono- or polysubstituted by halogen, OH, CN, CF₃, C₁₋₃-alkyl, or hydroxy-C₁₋₃-alkyl;
R¹³ is independently R¹⁷;
R¹⁵ is H, C₁₋₄-alkyl, C₃₋₇-cycloalkyl, C₃₋₇-cycloalkyl-C₁₋₃-alkyl, phenyl, phenyl-C₁₋₃-alkyl, pyridinyl, or pyridinyl-C₁₋₃-alkyl;
R¹⁶ is H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl, or cyclo-C₃₋₆-alkyleneimino-C₂₋₆-alkyl;
R¹⁷ is independently R¹⁶ or phenyl, phenyl-C₁₋₃-alkyl, pyridinyl, C₁₋₄-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylsulfonyl, C₁₋₄-alkylsulfonylamino-C₂₋₃-alkyl, or N—(C₁₋₄-alkylsulfonyl)-N(-C₁₋₄-alkyl)-amino-C₂₋₃-alkyl;
R¹⁸ and R¹⁹ are each independently H or C₁₋₆-alkyl;
R²⁰ is halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl, or R²²;
R²¹ is C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulfonyl, aminosulfonyl, C₁₋₄-alkylaminosulfonyl, di-C₁₋₄-alkylaminosulfonyl, or cyclo-C₃₋₆-alkylene-iminosulfonyl; and
R²² is pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-aminocarbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonylamino, cyclo-C₃₋₆-alkyleneimino, phenyl-C₁₋₃-alkylamino, N—(C₁₋₄-alkyl)phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino, or C₁₋₄-alkylaminocarbonylamino,
wherein in each of the abovementioned groups and residues one or more C atoms are additionally optionally mono- or polysubstituted by F and/or one or two C atoms are independently optionally monosubstituted by Cl or Br and/or one or more phenyl rings independently optionally contain one, two, or three substituents selected from F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl-, and di-(C₁₋₃-alkyl)-amino-C₁₋₃-alkyl and/or are optionally monosubstituted by nitro, and
the H atom of any carboxy group present or an H atom bound to an N atom are each optionally replaced by a group which can be cleaved in vivo, and
the tautomers, enantiomers, salts, and mixtures thereof,
and excluding the following compounds:
(2-{4-[5-(4-chlorophenyl)pyridin-2-ylethynyl]-2-methylphenoxy}ethyl)methylprop-2-ynylamine,
(2-{5-[5-(4-chlorophenyl)pyridin-2-ylethynyl]indol-1-yl}ethyl)cyclopropylmethylprop-2-ynylamine,
{4-[6-(4-chlorophenyl)quinolin-2-ylethynyl]benzyl}methyl(tetrahydropyran-4-yl)-amine,
allyl-(2-{4-[5-(4-chlorophenyl)pyridin-2-ylethynyl]phenoxy}ethyl)cyclopropylmethylamine,
allyl-(2-{4-[5-(4-chlorophenyl)pyridin-2-ylethynyl]-2-methylphenoxy}ethyl)cyclopropylmethyl-amine, and
allyl-(2-{5-[5-(4-chlorophenyl)pyridin-2-ylethynyl]indol-1-yl}ethyl)cyclopropylmethylamine.

2. The compound of formula (I) according to claim 1, wherein:
R¹ is C₃₋₆-alkenyl, C₃₋₆-alkynyl, (hydroxy-C₃₋₇-cycloalkyl)-C₁₋₃-alkyl, oxa-C₅₋₇-cycloalkyl, or dihydroxy-C₃₋₇-alkyl, each optionally independently mono- or polysubstituted by substituents selected from halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino, or di-(C₁₋₄-alkyl)-amino, wherein the alkyl, alkoxy, and cycloalkyl groups thereof optionally comprise one or more identical or different halogen or hydroxy substituents; and
R² is independently R¹ or is H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)pyrrolidin-3-yl, pyrrolidinyl-C₁₋₃-alkyl, N—(C₁₋₄-alkyl)pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, phenyl, phenyl-C₁₋₃-alkyl, pyridyl, or pyridyl-C₁₋₃-alkyl, wherein one or more C atoms thereof are optionally independently mono- or polysubstituted by F, C₁₋₃-alkyl, or hydroxy-C₁₋₃-alkyl, and/or one or two C atoms are optionally independently monosubstituted by Cl, Br, OH, CF₃, or CN, and the phenyl or pyridyl group are optionally independently mono- or polysubstituted by identical or different groups R²⁰ and/or monosubstituted by nitro.

3. The compound of formula (I) according to claim 1, wherein:
R¹ and R² together with the N atom to which they are bound is 3,4-dihydroxypyrrolidinyl, 3,4-dihydroxypiperidinyl, 3,5-dihydroxypiperidinyl, (hydroxy-C₁₋₃-alkyl)hydroxypyrrolidinyl, (hydroxy-C₁₋₃-alkyl)hydroxypiperidinyl, (hydroxy-C₃₋₆-cycloalkyl)hydroxypyrrolidinyl, (hydroxy-C₃₋₆-cycloalkyl)hydroxypiperidinyl, (C₁₋₃-alkyl-hydroxymethyl)pyrrolidinyl, (C₁₋₃-alkyl-hydroxymethyl)piperidinyl, (di-C₁₋₃-alkyl-hydroxymethyl)pyrrolidinyl, (di-C₁₋₃-alkyl-hydroxymethyl)piperidinyl, (1-hydroxy-C₃₋₆-cycloalkyl)pyrrolidinyl, or (1-hydroxy-C₃₋₆-cycloalkyl)piperidinyl, each optionally independently mono- or polysubstituted by substituents selected from halogen, hydroxy, cyano, C₁₋₄-alkyl, C₃₋₇-cycloalkyl, C₃₋₇-cycloalkyl-C₁₋₃-alkyl, C₁₋₄-alkoxy-C₁₋₄-alkyl, C₁₋₄-alkoxy, C₁₋₄-alkenyl, C₁₋₄-alkynyl, amino, C₁₋₄-alkyl-amino, and di-(C₁₋₄-alkyl)-amino, wherein the alkyl, alkoxy, and cycloalkyl groups thereof optionally comprise one or more identical or different halogen or hydroxy substituents.

4. The compound of formula (I) according to claim 1, wherein:
X is —CH₂—, ethylene, propylene, —CH₂—CH═CH—, —CH₂—C≡C—, —CH₂—CH₂—O—, —CH₂—CH₂—S—, or —CH₂—CH₂—NR⁴—, or
is C₂₋₄-alkylene having one or two identical or different substituents independently selected from fluorine, chlorine, hydroxy, and C₁₋₃-alkyl, and/or a C₂₋₆-alkenyl or cyclopropyl substituent, wherein two alkyl substituents are optionally joined together to form a C₃₋₆-cycloalkyl group or an alkyl and an alkenyl group are optionally joined together to form a C₅₋₆-cycloalkenyl group, or
—CH₂—CH═CH—, —CH₂—C≡C—, —CH₂—CH₂—O—, —CH₂—CH₂—S—, or —CH₂—CH₂—NR⁴—, each one or two identical or different substituents independently selected from fluorine and C₁₋₃-alkyl, and/or a cyclopropyl substituent, wherein two alkyl groups are optionally joined together to form a C₃₋₆-cycloalkyl group or, if an alkyl group is R⁴, they are optionally joined together to form a pyrrolidine or piperidine group.

5. The compound of formula (I) according to claim 1, wherein:
Z is a single bond or ethylene; and
W is a single bond.

6. The compound of formula (I) according to claim 1, wherein:
Y is phenyl, pyridinyl, pyrimidinyl, pyrazinyl, pyridazinyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, benzothienyl, or benzofuranyl, each optionally mono- or polysubstituted at one or more C atoms by identical or different groups R²⁰, and, in the case of a phenyl ring, is optionally additionally monosubstituted by nitro, and/or optionally substituted by R²¹ at one or more N atoms.

7. The compound of formula (I) according to claim 1, wherein:
A is phenyl, pyridinyl, pyrimidinyl, pyrazinyl, or pyridazinyl, each optionally mono- or polysubstituted at one or more C atoms by identical or different groups R²⁰, and, in the case of a phenyl ring, is optionally additionally monosubstituted by nitro.

8. The compound of formula (I) according to claim 1, wherein:
B is phenyl, cyclohexenyl, pyridyl, thienyl, or furanyl, each optionally mono- or polysubstituted at one or more C atoms by identical or different groups R²⁰, and, in the case of a phenyl group, is optionally additionally monosubstituted by nitro.

9. The compound of formula (I) according to claim 1, wherein: 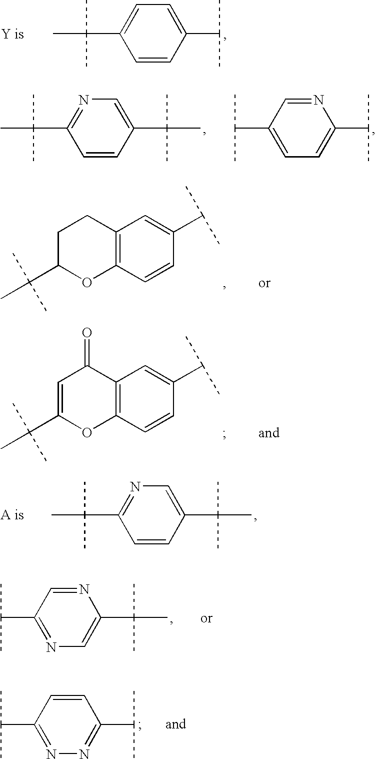
B is phenyl, cyclohexenyl, pyridyl, thienyl, or furanyl,
wherein Y and A are each optionally independently monosubstituted by R²⁰, and B is optionally independently mono-, di-, or trisubstituted by R²⁰, and, in the case of a phenyl ring, is optionally additionally monosubstituted by nitro.

10. The compound of formula (I) according to claim 1, wherein:
R²⁰ is F, Cl, Br, I, OH, cyano, amino, methyl, difluoromethyl, trifluoromethyl, ethyl, n-propyl, isopropyl, acetyl, methoxy, difluoromethoxy, trifluoromethoxy, ethoxy, n-propoxy, or isopropoxy, wherein substituents R²⁰ that occur repeatedly have identical or different meanings.

11. A physiologically acceptable salt of the compound according to claim 1.

12. A pharmaceutical formulation comprising the compound according to claim 1 and one or more physiologically acceptable excipients or inert carriers or diluents.

13. A pharmaceutical formulation comprising the compound according to claim 2 and one or more physiologically acceptable excipients or inert carriers or diluents.

14. A pharmaceutical formulation comprising the compound according to claim 3 and one or more physiologically acceptable excipients or inert carriers or diluents.

15. A pharmaceutical formulation comprising the physiologically acceptable salt according to claim 11 and one or more physiologically acceptable excipients or inert carriers or diluents.

16. The pharmaceutical formulation according to claim 12 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

17. The pharmaceutical formulation according to claim 13 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

18. The pharmaceutical formulation according to claim 14 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

19. The pharmaceutical formulation according to claim 15 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

20. A method for influencing the eating behavior of a mammal comprising administering to the mammal an effective amount of the compound according to claim 1.

21. A method for influencing the eating behavior of a mammal comprising administering to the mammal an effective amount of the compound according to claim 2.

22. A method for influencing the eating behavior of a mammal comprising administering to the mammal an effective amount of the compound according to claim 3.

23. A method for influencing the eating behavior of a mammal comprising administering to the mammal an effective amount of the physiologically acceptable salt according to claim 11.

24. A method for reducing the body weight and/or for preventing an increase in the body weight of a mammal comprising administering to the mammal an effective amount of the compound according to claim 1.

25. A method for reducing the body weight and/or for preventing an increase in the body weight of a mammal comprising administering to the mammal an effective amount of the compound according to claim 2.

26. A method for reducing the body weight and/or for preventing an increase in the body weight of a mammal comprising administering to the mammal an effective amount of the compound according to claim 3.

27. A method for reducing the body weight and/or for preventing an increase in the body weight of a mammal comprising administering to the mammal an effective amount of the physiologically acceptable salt according to claim 11.

28. A method for preventing or treating a metabolic disorder or eating disorder in a mammal comprising administering to the mammal an effective amount of the compound according to claim 1.

29. A method for preventing or treating a metabolic disorder or eating disorder in a mammal comprising administering to the mammal an effective amount of the compound according to claim 2.

30. A method for preventing or treating a metabolic disorder or eating disorder in a mammal comprising administering to the mammal an effective amount of the compound according to claim 3.

31. A method for preventing or treating a metabolic disorder or eating disorder in a mammal comprising administering to the mammal an effective amount of the physiologically acceptable salt according to claim 11.

32. The method according to claim 28, wherein the metabolic disorder or eating disorder is obesity, bulimia, bulimia nervosa, cachexia, anorexia, anorexia nervosa, or hyperphagia.

33. The method according to claim 29, wherein the metabolic disorder or eating disorder is obesity, bulimia, bulimia nervosa, cachexia, anorexia, anorexia nervosa, or hyperphagia.

34. The method according to claim 30, wherein the metabolic disorder or eating disorder is obesity, bulimia, bulimia nervosa, cachexia, anorexia, anorexia nervosa, or hyperphagia.

35. The method according to claim 31, wherein the metabolic disorder or eating disorder is obesity, bulimia, bulimia nervosa, cachexia, anorexia, anorexia nervosa, or hyperphagia.

36. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 1.

37. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 2.

38. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 3.

39. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 1.

40. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 2.

41. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 3.

42. A method for preventing or treating urinary incontinence, hyperactive urinary bladder, urgency, nycturia, or enuresis in a mammal comprising administering to the mammal an effective amount of the compound according to claim 1.

43. A method for preventing or treating urinary incontinence, hyperactive urinary bladder, urgency, nycturia, or enuresis in a mammal comprising administering to the mammal an effective amount of the compound according to claim 2.

44. A method for preventing or treating urinary incontinence, hyperactive urinary bladder, urgency, nycturia, or enuresis in a mammal comprising administering to the mammal an effective amount of the compound according to claim 3.

45. A method for treating dependencies and/or withdrawal symptoms in a mammal comprising administering to the mammal an effective amount of the compound according to claim 1.

46. A method for treating dependencies and/or withdrawal symptoms in a mammal comprising administering to the mammal an effective amount of the compound according to claim 2.

47. A method for treating dependencies and/or withdrawal symptoms in a mammal comprising administering to the mammal an effective amount of the compound according to claim 3.
